SEQUENCE LISTING (18 sequences)

SEQ ID NO: 1 - protein, 871 aa, Homo sapiens
MADSSEGPRAGPGEVAELPGDESGTPGGEAFPLSSLANLFEGEDGSLSPSPADASRPAGPGDGRPNLRMKFQGAFRKGVPNPIDLLESTLYESSVVPGPKKAPMDSLFDYGTYRHHSSDNKRWRKKIIEKQPQSPKAPAPQPPPILKVFNRPILFDIVSRGSTADLDGLLPFLLTHKKRLTDEEFREPSTGKTCLPKALLNLSNGRNDTIPVLLDIAERTGNMREFINSPFRDIYYRGQTALHIAIERRCKHYVELLVAQGADVHAQARGRFFQPKDEGGYFYFGELPLSLAACTNQPHIVNYLTENPHKKADMRRQDSRGNTVLHALVAIADNTRENTKFVTKMYDLLLLKCARLFPDSNLEAVLNNDGLSPLMMAAKTGKIGIFQHIIRREVTDEDTRHLSRKFKDWAYGPVYSSLYDLSSLDTCGEEASVLEILVYNSKIENRHEMLAVEPINELLRDKWRKFGAVSFYINVVSYLCAMVIFTLTAYYQPLEGTPPYPYRTTVDYLRLAGEVITLFTGVLFFFTNIKDLFMKKCPGVNSLFIDGSFQLLYFIYSVLVIVSAALYLAGIEAYLAVMVFALVLGWMNALYFTRGLKLTGTYSIMIQKILFKDLFRFLLVYLLFMIGYASALVSLLNPCANMKVCNEDQTNCTVPTYPSCRDSETFSTFLLDLFKLTIGMGDLEMLSSTKYPVVFIILLVTYIILTFVLLLNMLIALMGETVGQVSKESKHIWKLQWATTILDIERSFPVFLRKAFRSGEMVTVGKSSDGTPDRRWCFRVDEVNWSHWNQNLGIINEDPGKNETYQYYGFSHTVGRLRRDRWSSVVPRVVELNKNSNPDEVVVPLDSMGNPRCDGHQQGYPRKWRTDDAPL

SEQ ID NO: 2 - DNA, 3228 bp, Homo sapiens
attcaggaagcgcggatctcccggccgccggcgcccagccgtcccggaggctgagcagtgcagacgggcctggggcaggcatggcggattccagcgaaggcccccgcgcggggcccggggaggtggctgagctccccggggatgagagtggcaccccaggtggggaggcttttcctctctcctccctggccaatctgtttgagggggaggatggctccctttcgccctcaccggctgatgccagtcgccctgctggcccaggcgatgggcgaccaaatctgcgcatgaagttccagggcgccttccgcaagggggtgcccaaccccatcgatctgctggagtccaccctatatgagtcctcggtggtgcctgggcccaagaaagcacccatggactcactgtttgactacggcacctatcgtcaccactccagtgacaacaagaggtggaggaagaagatcatagagaagcagccgcagagccccaaagcccctgcccctcagccgccccccatcctcaaagtcttcaaccggcctatcctctttgacatcgtgtcccggggctccactgctgacctggacgggctgctcccattcttgctgacccacaagaaacgcctaactgatgaggagtttcgagagccatctacggggaagacctgcctgcccaaggccttgctgaacctgagcaatggccgcaacgacaccatccctgtgctgctggacatcgcggagcgcaccggcaacatgagggagttcattaactcgcccttccgtgacatctactatcgaggtcagacagccctgcacatcgccattgagcgtcgctgcaaacactacgtggaacttctcgtggcccagggagctgatgtccacgcccaggcccgtgggcgcttcttccagcccaaggatgaggggggctacttctactttggggagctgcccctgtcgctggctgcctgcaccaaccagccccacattgtcaactacctgacggagaacccccacaagaaggcggacatgcggcgccaggactcgcgaggcaacacagtgctgcatgcgctggtggccattgctgacaacacccgtgagaacaccaagtttgttaccaagatgtacgacctgctgctgctcaagtgtgcccgcctcttccccgacagcaacctggaggccgtgctcaacaacgacggcctctcgcccctcatgatggctgccaagacgggcaagattgggatctttcagcacatcatccggcgggaggtgacggatgaggacacacggcacctgtcccgcaagttcaaggactgggcctatgggccagtgtattcctcgctttatgacctctcctccctggacacgtgtggggaagaggcctccgtgctggagatcctggtgtacaacagcaagattgagaaccgccacgagatgctggctgtggagcccatcaatgaactgctgcgggacaagtggcgcaagttcggggccgtctccttctacatcaacgtggtctcctacctgtgtgccatggtcatcttcactctcaccgcctactaccagccgctggagggcacaccgccgtacccttaccgcaccacggtggactacctgcggctggctggcgaggtcattacgctcttcactggggtcctgttcttcttcaccaacatcaaagacttgttcatgaagaaatgccctggagtgaattctctcttcattgatggctccttccagctgctctacttcatctactctgtcctggtgatcgtctcagcagccctctacctggcagggatcgaggcctacctggccgtgatggtctttgccctggtcctgggctggatgaatgccctttacttcacccgtgggctgaagctgacggggacctatagcatcatgatccagaagattctcttcaaggaccttttccgattcctgctcgtctacttgctcttcatgatcggctacgcttcagccctggtctccctcctgaacccgtgtgccaacatgaaggtgtgcaatgaggaccagaccaactgcacagtgcccacttacccctcgtgccgtgacagcgagaccttcagcaccttcctcctggacctgtttaagctgaccatcggcatgggcgacctggagatgctgagcagcaccaagtaccccgtggtcttcatcatcctgctggtgacctacatcatcctcacctttgtgctgctcctcaacatgctcattgccctcatgggcgagacagtgggccaggtctccaaggagagcaagcacatctggaagctgcagtgggccaccaccatcctggacattgagcgctccttccccgtattcctgaggaaggccttccgctctggggagatggtcaccgtgggcaagagctcggacggcactcctgaccgcaggtggtgcttcagggtggatgaggtgaactggtctcactggaaccagaacttgggcatcatcaacgaggacccgggcaagaatgagacctaccagtattatggcttctcgcataccgtgggccgcctccgcagggatcgctggtcctcggtggtaccccgcgtggtggaactgaacaagaactcgaacccggacgaggtggtggtgcctctggacagcatggggaacccccgctgcgatggccaccagcagggttacccccgcaagtggaggactgatgacgccccgctctagggactgcagcccagccccagcttctctgcccactcatttctagtccagccgcatttcagcagtgccttctggggtgtccccccacaccctgctttggccccagaggcgagggaccagtggaggtgccagggaggccccaggaccctgtggtcccctggctctgcctccccaccctggggtgggggctcccggccacctgtcttgctcctatggagtcacataagccaacgccagagcccctccacctcaggccccagcccctgcctctccattatttatttgctctgctctcaggaagcgacgtgacccctgccccagctggaacctggcagaggccttaggaccccgttccaagtgcactgcccggccaagccccagcctcagcctgcgcctgagctgcatgcgccaccatttttggcagcgtggcagctttgcaaggggctggggccctcggcgtggggccatgccttctgtgtgttctgtagtgtctgggatttgccggtgctcaataaatgtttattcattgacggtg

SEQ ID NO: 3 - DNA, 99 bp, Homo sapiens
ccgatgtgtatcctcagctttgagaactgaattccatgggttgtgtcagtgtcagacctctgaaattcagttcttcagctgggatatctctgtcatcgt

SEQ ID NO: 4 - DNA, 19 bp, Artificial Sequence
  Synthetic
gagaactgaattccatggg

SEQ ID NO: 5 - DNA, 19 bp, Artificial Sequence
  Synthetic
gaggtagtaggttgtgtgg

SEQ ID NO: 6 - DNA, 18 bp, Artificial Sequence
  Synthetic
ccctgagaccctaacttg

SEQ ID NO: 7 - DNA, 20 bp, Artificial Sequence
  Synthetic
gtggttcttgacagttcaac

SEQ ID NO: 8 - DNA, 20 bp, Artificial Sequence
  Synthetic
agcagcacgtaaatattggc

SEQ ID NO: 9 - RNA, 25 nt, Artificial Sequence
  Synthetic
guagaaucaaguuucuaauucugaa

SEQ ID NO: 10 - RNA, 27 nt, Artificial Sequence
  Synthetic
uucagaauuagaaacuugauucuacca

SEQ ID NO: 11 - RNA, 25 nt, Artificial Sequence
  Synthetic
agcuaaaacugagagcuucaaacag

SEQ ID NO: 12 - RNA, 27 nt, Artificial Sequence
  Synthetic
cuguuugaagcucucaguuuuagcuua

SEQ ID NO: 13 - DNA, 23 bp, Artificial Sequence
  Synthetic
cctgccttttcgtctctagccgc

SEQ ID NO: 14 - DNA, 24 bp, Artificial Sequence
  Synthetic
gctgatgacctcccagaacttggc

SEQ ID NO: 15 - DNA, 25 bp, Artificial Sequence
  Synthetic
gtgggcaagagctcagatggcactc

SEQ ID NO: 16 - DNA, 24 bp, Artificial Sequence
  Synthetic
ccaccgaggaccaacgatccctac

SEQ ID NO: 17 - protein, 871 aa, Rattus norvegicus
MADPGDGPRAAPGDVAEPPGDESGTSGGEAFPLSSLANLFEGEEGSSSLSPVDASRPAGPGDGRPNLRMKFQGAFRKGVPNPIDLLESTLYESSVVPGPKKAPMDSLFDYGTYRHHPSDNKRWRRKVVEKQPQSPKAPAPQPPPILKVFNRPILFDIVSRGSTADLDGLLSYLLTHKKRLTDEEFREPSTGKTCLPKALLNLSNGRNDTIPVLLDIAERTGNMREFINSPFRDIYYRGQTALHIAIERRCKHYVELLVAQGADVHAQARGRFFQPKDEGGYFYFGELPLSLAACTNQPHIVNYLTENPHKKADMRRQDSRGNTVLHALVAIADNTRENTKFVTKMYDLLLLKCSRLFPDSNLETVLNNDGLSPLMMAAKTGKIGVFQHIIRREVTDEDTRHLSRKFKDWAYGPVYSSLYDLSSLDTCGEEVSVLEILVYNSKIENRHEMLAVEPINELLRDKWRKFGAVSFYINVVSYLCAMVIFTLTAYYQPLEGTPPYPYRTTVDYLRLAGEVITLLTGVLFFFTSIKDLFMKKCPGVNSLFVDGSFQLLYFIYSVLVVVSAALYLAGIEAYLAVMVFALVLGWMNALYFTRGLKLTGTYSIMIQKILFKDLFRFLLVYLLFMIGYASALVTLLNPCTNMKVCNEDQSNCTVPSYPACRDSETFSAFLLDLFKLTIGMGDLEMLSSAKYPVVFILLLVTYIILTFVLLLNMLIALMGETVGQVSKESKHIWKLQWATTILDIERSFPVFLRKAFRSGEMVTVGKSSDGTPDRRWCFRVDEVNWSHWNQNLGIINEDPGKSEIYQYYGFSHTMGRLRRDRWSSVVPRVVELNKNSGTDEVVVPLDNLGNPNCDGHQQGYAPKWRAEDAPL

SEQ ID NO: 18 - DNA, 3211 bp, Rattus norvegicus
gggaggaggacgcggcgggatcaggaagcggctgcgctgcgcccgcgtcccaagcaggccgagaagtccaaacagatctgctcagggtccagtatggcagatcctggtgatggcccccgtgcagcgcctggggatgtggctgagccccctggagacgagagtggcacttctggtggggaggccttccccctctcttccctggccaacctgtttgagggagaggaaggctcctcttctctttcaccagtggatgctagccgccctgctggccccggggatggacgtccaaacctgcgtatgaagttccagggcgctttccgcaagggggttcccaaccccattgacctgctggagtccaccctgtatgagtcctcagtagtgcctgggcccaagaaagcgcccatggattcgttgttcgactatggcacttaccggcaccaccccagtgacaacaagagatggaggaggaaggtcgtagagaagcagccacagagccccaaagctcccgccccccagccaccccccatcctcaaagtcttcaaccggcccatcctctttgacatcgtgtcccggggctccactgccgacctggacggactgctctcctacttgctgacccacaagaagcgcctgactgatgaggagttccgggaaccatccacagggaagacctgcctgcccaaggcacttctgaacttaagcaatggccgaaacgacaccatcccagtgttgctggacattgcggaacgcacgggcaacatgcgggagttcatcaactcgcccttcagagacatctactaccgagggcagacggcactgcacatcgccattgaacggcgctgcaagcattacgtggagctcctggtggcccagggagccgatgtgcacgcgcaggcccgagggcggttcttccagcccaaggatgagggtggctacttctactttggggagctgcccttgtccttggcagcctgcaccaaccagccgcacatcgtcaactacctgacagagaaccctcacaagaaagccgatatgaggcgacaggactccagaggcaacacggtgctccacgcgctggtggccatcgctgacaacacccgagagaacaccaagtttgtcaccaagatgtatgacctgttgcttctcaagtgctcccgcctcttcccagacagcaacctggagactgtgcttaacaatgacggtctttcgcccctcatgatggctgccaagactggcaagatcggggtctttcagcacatcatccgacgggaggtgacagatgaggacacacggcacctgtctcgcaagttcaaggactgggcctacgggcctgtgtattcttctctctacgacctctcctccctggatacgtgcggggaggaagtgtccgtgctggagatcctggtttacaacagcaagatcgagaaccgccatgagatgctggctgtggagcccattaacgaactgctgagggacaagtggcgtaagttcggggccgtgtccttctacatcaacgttgtctcctatctgtgtgccatggtcatcttcaccctcacagcctactatcagccactggagggcacgccaccctacccttaccgtaccacggtggactacctgaggctggctggtgaggtcatcacgctcctcacaggagtcctgttcttctttaccagtatcaaagacttgttcatgaagaaatgccctggagtgaattctctcttcgtcgatggctccttccagttgctctacttcatctactcagtgctggtggttgtgtctgcggcgctctacctggcagggatcgaggcctatctggctgtgatggtctttgccctggtcctgggctggatgaatgccctttacttcacccgtgggctgaagctgacagggacctacagcatcatgattcagaagatcctcttcaaagatctcttccgctttctgctggtctacctgctttttatgattggctatgcctcagctctggtcaccctcctgaatccgtgcaccaacatgaaggtctgtaacgaggaccagagcaactgcacggtgccctcataccccgcgtgccgggacagcgagaccttcagcgccttcctactggacctcttcaagctcaccatcggcatgggcgacctggagatgctgagcagcgctaagtaccccgtggtcttcattctcctgctggttacctacatcatcctcaccttcgtgctcctgctgaacatgctcatcgccctcatgggtgagaccgtgggccaggtgtccaaggagagcaagcacatctggaagctgcagtgggccaccaccatcctggacatcgagcgctccttccctgtgttcctgaggaaggccttccgctccggagagatggtgacagtgggcaagagctcggatggcactccagaccgcaggtggtgcttcagggtggacgaggtgaactggtctcactggaaccagaacctgggcatcattaacgaggaccccggcaagagcgagatctaccagtactatggcttctcccataccatggggcgcctccgcagggatcgctggtcctcagtggtgccccgcgtggtggagctgaacaagaactcaggcacagatgaagtggtggtccccctggataacctagggaaccccaactgtgacggccaccagcaaggttatgctcccaagtggagggcggaggacgcaccactgtaggggccatgccagggctggggtcaatggcccaggcttggcccttgctcccacctacatttcagcatctgtcctgtgtcttcccacacccacacgtgacctcggaggtgagggcctctgtggagactctggggaggccccaggaccctctggtccccacaaagacttttgctcttatttctactcctccccacatgggggacggggctcctggccacctgtctcactcccatggagtcacctaagccagctcagggcccctccactcacagggctcaggcccctgtccctcttgtgcactatttattgctctcctcaggaaaatgacatcacaggagtctacctgcagctggaacctggccagggctgaggctcatgcagggacactgcagccctgacccgctgcagatctgacctgctgcagcccgggctagggtgggtcttctgtactttgtagagatcggggctgttggtgctcaataaatgtttgtttattctcggtgga

(19) United States
(12) Patent Application Publication
(10) Pub. No.: US 2024/0002846 A1
(43) Pub. Date: Jan. 4, 2024

(54) COMPOSITIONS AND METHODS FOR THE DIAGNOSIS AND TREATMENT OF ITCH

(71) Applicants: Duke University, Durham, NC (US); Universidad Nacional Autónoma de México, Ciudad de (MX); Wake Forest University Health Sciences, Winston-Salem, NC (US)

(72) Inventors: Wolfgang Liedtke, Durham, NC (US); Yong Chen, Durham, NC (US); Tamara Rosenbaum, Ciudad de México (MX); Tony Elvern Reeves, Winston-Salem, NC (US)

(21) Appl. No.: 18/032,103

(22) PCT Filed: Oct. 15, 2021
(86) PCT No.: PCT/US2021/055288

§ 371 Date: Apr. 14, 2023

Related U.S. Application Data
(60) Provisional application No. 63/091,973, filed on Oct. 15, 2020.

(51) Int. Cl. C12N 15/113; G01N 33/68; A61P 17/04

(52) U.S. Cl. CPC C12N 15/113; G01N 33/6872; G01N 33/6893; A61P 17/04; G01N 2800/20; G01N 2500/04; C12N 2310/141

(57) ABSTRACT

Disclosed herein are compositions and methods for treating a subject having an itch-related disorder, such as dermatological disorders or systemic disorders comprising itch. The methods may include determining the level of a biomarker in a biological sample from the subject. The biomarker may be selected from TRPV4 expression, lysophosphatidylcholine, and miRNA-146a expression, or a combination thereof. The subject may be identified as having the itch-related disorder when the level of the biomarker is greater in a sample from the subject than in a control. An anti-pruritic therapy may be administered to treat the subject having the itch-related disorder.

### CROSS-REFERENCE TO RELATED APPLICATIONS

This application claims priority to U.S. Provisional Patent Application No. 63/091,973 filed Oct. 15, 2020, which is incorporated herein by reference in its entirety.

### FIELD

This disclosure relates to diagnostic and treatment methods for itch-related disorders.

### STATEMENT REGARDING FEDERALLY SPONSORED RESEARCH

This invention was made with government support under grant DE018549 awarded by the National Institutes of Health/National Institute of dental and Craniofacial Research, grant K12DE022793 awarded by the National Institutes of Health, and grant R01 DE027454 awarded by the National Institutes of Health. The government has certain rights in the invention. This invention was also made with government support under grant A1-S-8760 from Consejo Nacional de Ciencia y Tecnologia.

### INTRODUCTION

Treatment for itch (pruritus) associated with systemic disorders represents a severe unmet medical need for patients with cholestatic liver disease, chronic kidney disease, cancers, infectious diseases, side effects of treatment, and some forms of lymphoproliferative disorders. Cholestatic itch is a debilitating symptom which has significant prevalence in patients with hepatobiliary diseases, such as primary biliary cholangitis (PBC), primary sclerosing cholangitis (PSC), and intrahepatic cholestasis of pregnancy (ICP). Therapeutic recourse is dire because the underlying pathophysiology remains largely elusive.

### SUMMARY

In an aspect, the disclosure relates to a method of treating a subject having an itch-related disorder. The method may include determining the level of a biomarker in a biological sample from the subject, wherein the biomarker is selected from TRPV4 expression, lysophosphatidylcholine, and miRNA-146a expression, or a combination thereof; and administering an anti-pruritic therapy to treat the subject identified as having the itch-related disorder, wherein the subject is identified as having the itch-related disorder when the level of the biomarker is greater in the biological sample than in a control sample.

In a further aspect, the disclosure relates to a method of treating an itch-related disorder in a subject. The method may include (a) determining the level of a biomarker in a biological sample from the subject, wherein the biomarker is selected from TRPV4 expression, lysophosphatidylcholine, and miRNA-146a expression, or a combination thereof, and wherein the level of the biomarker is greater in the biological sample than in a control sample; (b) diagnosing the subject as having an itch-related disorder based on the level of the biomarker determined in step (a); and (c) administering an anti-pruritic therapy to the subject diagnosed as having an itch-related disorder in step (b).

Another aspect of the disclosure provides a method of diagnosing an itch-related disorder in a subject. The method may include determining the level of a biomarker in a biological sample from the subject, wherein the biomarker is selected from TRPV4 expression, lysophosphatidylcholine, and miRNA-146a expression, or a combination thereof; and diagnosing the subject as having an itch-related disorder when the level of the biomarker is greater in the biological sample than in a control sample. In some embodiments, the method further comprises administering an anti-pruritic therapy to the subject diagnosed as having an itch-related disorder.

In some embodiments, the itch-related disorder comprises itch.

Another aspect of the disclosure provides a method of identifying an itch-related disorder in a subject. The method may include (i) obtaining a biological sample from the subject; (ii) identifying the presence of a biomarker in the subject, the biomarker selected from the group consisting of TRPV4, miRNA-146a, lysophosphatidylcholine, and combinations thereof; (iii) quantifying the expression level of the biological sample, in which the presence of one or more of the biomarkers in an amount greater than the control is indicative of the itch-related disorder comprising itch; and (iv) administering to the subject an appropriate anti-pruritic therapy if the level of biomarker is greater in the biological sample than in a control sample.

In some embodiments, the biomarker is the level of TRPV4 expression. In some embodiments, the biomarker is the level of lysophosphatidylcholine. In some embodiments, the biomarker is the level of miRNA-146a expression. In some embodiments, the itch-related disorder is a dermatological disorder or a systemic disorder. In some embodiments, the itch-related disorder is a systemic disorder selected from liver disorder, kidney disorder, cancer, lymphoma, infection, or medication side-effect. In some embodiments, the itch-related disorder is selected from the group consisting of cholestatic itch, uremic itch, pruritic psoriasis, and combinations thereof. In some embodiments, the level of TRPV4 expression or the level of miRNA-146a expression is an RNA expression level. In some embodiments, the level of the biomarker is determined by microarray analysis, or PCR, or a combination thereof. In some embodiments, the control sample is from a healthy subject. In some embodiments, the biological sample comprises skin. In some embodiments, the biological sample comprises skin keratinocytes. In some embodiments, the biological sample comprises blood. In some embodiments, the anti-pruritic therapy is selected from the group consisting of moisturizers, capsaicin, salicylic acid, emollients, topical corticosteroids, topical calcineurin inhibitors, antihistamines, menthol, local anesthetics, cannabinoids, immunomodulators, antihistamines, antidepressants, μ-opiod receptor agonists, k-opiod receptor agonists, neuroleptics, substance P antagonist, immunosuppressants, methylnaltrexone, NGX-4010, TS-022, Serine proteases/PAR2 antagonists, IL-31 antibody, IL-4-receptor antibody, IL-13 antibody, TSLP-antibody, IL-5 antibody, and combinations thereof. In some embodiments, the anti-pruritic therapy comprises an immunomodulator. In some embodiments, the immunomodulator comprises a TRPV4 inhibitor. In some embodiments, the subject is a mammal.

In some embodiments, the anti-pruritic therapy comprises a TRPV4 inhibitor. In some embodiments, the TRPV4 inhibitor binds to a C-terminal region of TRPV4. In some embodiments, the TRPV4 inhibitor binds at least one amino acid in a motif comprising K750-W772 of Xenopus TRPV4 or K754-W776 of mammalian TRPV4 or an amino acid corresponding thereto. In some embodiments, the TRPV4 inhibitor binds at least one amino acid in a motif comprising R742-W772 of Xenopus TRPV4 or R746-W776 of mammalian TRPV4 or an amino acid corresponding thereto. In some embodiments, the TRPV4 inhibitor binds at least one amino acid in a motif comprising K750-W772 and R742 of Xenopus TRPV4 or K754-W776 and R746 of mammalian TRPV4 or an amino acid corresponding thereto. In some embodiments, the TRPV4 inhibitor binds Arg-746 of mammalian TRPV4 or Arg-742 of Xenopus TRPV4 or an amino acid corresponding thereto. In some embodiments, the TRPV4 inhibitor binds at least one amino acid selected from K754, R757, R774, and W776 of mammalian TRPV4 or an amino acid corresponding thereto.

Another aspect of the disclosure provides a method of screening for a compound that modulates TRPV4. The method may include testing a plurality of compounds for binding to wild-type TRPV4 to determine from the plurality of compounds a subset of compounds that bind wild-type TRPV4; and testing the subset of compounds that bind wild-type TRPV4 for binding to at least one mutant TRPV4, wherein the mutant TRPV4 comprises a mutation of at least one amino acid in the motif corresponding to K746-W776 of mammalian TRPV4, or an amino acid corresponding thereto, to determine from the subset of compounds a compound that binds wild-type TRPV4 but not the mutant TRPV4. In some embodiments, at least one amino acid in the motif corresponding to K754-W776 of mammalian TRPV4 is mutated to an alanine. In some embodiments, at least one amino acid in the motif corresponding to K754-W776 of mammalian TRPV4 is mutated to a glycine. In some embodiments, at least one amino acid selected from K754, R757, R774, and W776 of mammalian TRPV4, or an amino acid corresponding thereto, is mutated. In some embodiments, the mutant TRPV4 has activity as an ion channel. In some embodiments, the method further comprises determining the effect of the compound that binds wild-type TRPV4 but not the mutant TRPV4 on the activity of wild-type TRPV4. In some embodiments, the compound that binds wild-type TRPV4 but not the mutant TRPV4 inhibits the activity of wild-type TRPV4. In some embodiments, the compound that binds wild-type TRPV4 but not the mutant TRPV4 increases the activity of wild-type TRPV4. In some embodiments, the compound that binds wild-type TRPV4 but not the mutant TRPV4 inhibits or reduces the binding of LCP to wild-type TRPV4.

The disclosure provides for other aspects and embodiments that will be apparent in light of the following detailed description and accompanying figures.

### BRIEF DESCRIPTION OF THE DRAWINGS

FIG. 1A-FIG. 1P. LPC-induced, but not LPA-induced scratching behavior requires Trpv4 in skin keratinocytes. (FIG. 1A) I.d. injection of LPA (18:1) induced itch that was not significantly altered in any of the mouse lines tested: Trpv4 KO, wild type (WT) mice i.p. pretreated with TRPV4 inhibitors GSK205 (20 mg/kg) or HC067047 (20 mg/kg), sensory neuron-Trpv4 cKO (Nav1.8-Cre::Trpv4^{fl/fl}), or keratinocyte-Trpv4 cKO (K14-Cre::Trpv4^{fl/fl}, tamoxifen (tam)-inducible). n=4-7 mice/group. (FIG. 1B) I.d. injection of LPC (egg-LPC, a mixture of LPC species, see Example 1), the precursor of LPA, elicited a dose-dependent itch. **p<0.01 and ***p<0.001 vs. normal saline (NS), n=5 mice for NS and 50 μg, 6 mice for 150 μg, and 11 mice for 500 μg. (FIG. 1C) The mouse cheek model demonstrated that i.d. injection of LPC into the cheek elicited a robust scratching response (indicative of itch), not a wiping response (indicative of pain). ***p<0.001 vs. NS, n=4-5 mice/group. (FIG. 1D) I.d. injection of LPC induced itch that was not significantly attenuated in Trpv4 KO or in sensory neuron-Trpv4 cKO mice. In contrast, it was significantly reduced in WT mice i.p. pretreated with TRPV4 inhibitors GSK205 (20 mg/kg) or HC067 (20 mg/kg) and in keratinocyte-Trpv4 cKOs treated with tam. *p<0.05 and **p<0.01 vs. WT LPC, n=7-11 mice/group. (FIG. 1E) Different species of LPC: 14:0, 16:0, 18:0, and 18:1 also caused robust itch after i.d. administration, with LPC 18:1 most potent at the same dose (n=5 mice for all species except for 11 mice for LPC). (FIG. 1F) A major species of LPC (18:1)-caused itch was also significantly reduced in keratinocyte-Trpv4 cKOs treated with tam. ***p<0.001 vs. WT, n=4-5 mice/group. (FIG. 1G) I.t. injection of LPC did not elicit itch. n=5 mice/group. (FIG. 1H) LPC-induced itch was attenuated in WT mice by i.p. pretreatment with autotaxin (the enzyme converting LPC to LPA) inhibitor PF8380 (10 mg/kg), and this attenuation was further augmented in keratinocyte-Trpv4 cKOs. **p<0.01 and #p<0.05, n=8-11 mice/group. (FIG. 1I-FIG. 1L) LPC triggered Ca²⁺ influx in a dose-dependent manner in mouse (FIG. 1I) and human (FIG. 1K) keratinocytes (KC). LPC (10 μM) induced Ca²⁺ signal was significantly reduced by inhibition of TRPV4 with selective inhibitors GSK205 or HC067047 (both at 10 μM, FIG. 1J and FIG. 1L) and in keratinocyte-Trpv4cKO keratinocytes (J). *p<0.05, **p<0.01 and ***p<0.001 vs. LPC, n≥180 cells recorded/treatment. (FIG. 1M-FIG. 1P) LPA (18:1) induced Ca²⁺ influx was not dose-dependent and was less robust than that of LPC in mouse (FIG. 1M) and human (FIG. 10) keratinocytes (KC). In addition, the LPA 18:1 (10 μM) induced Ca²⁺ signal remained unchanged by inhibition of TRPV4 with GSK205 or HC067047 (10 μM, FIG. 1N and FIG. 1P) or in keratinocytes derived from keratinocyte-Trpv4 cKO (N). n≥200 cells recorded/treatment. One-way ANOVA with Tukey's post-test was used for FIG. 1A, FIG. 1B, FIG. 1D, FIG. 1F-FIG. 1H, FIG. 1J, FIG. 1L, FIG. 1N, and FIG. 1P, and two-tailed t-test for FIG. 1C.

FIG. 2A-FIG. 2G. LPC directly activates TRPV4 channels. (FIG. 2A-FIG. 2B) Representative current-time plots from excised inside-out membrane patches in HEK cells transfected with rTRPV4 are shown. These were obtained initially in the absence of agonist stimulation (gray), in the presence of 5 μM LPC 18:1 (blue) or 1 μM GSK1016790A (GSK101, black). (FIG. 2A) Traces shown were recorded at −60 and 60 mV. (FIG. 2B) Current-voltage relationships from −120 to 120 mV. n=5 cells/condition. (FIG. 2C) Single-channel recordings (left) of hTRPV4 activated with 100 nM GSK101 or 5 μM LPC 18:1 at +60 mV. The 0 and C refer to open and closed state of the channel. All-point histograms obtained from the traces (right). The average for the open level amplitudes was 6.45±0.55 pA with an open probability of 0.85±0.08 for GSK1016790A and 3.27±0.41 pA and 0.75±0.08 for LPC 18:1. n=5 cells/condition. (FIG. 2D-FIG. 2G) Activation of hTRPV4 channels by LPC 18:1 remained unchanged with mutations for PIP2 interaction sites. (FIG. 2D-FIG. 2F) Representative currents for hTRPV4, hTRPV4-R269H, and hTRPV4-121AAWAA125 channels. Currents were obtained initially in the absence of agonist (light grey), in the presence of 5 μM LPC 18:1, or in the presence of 1 μM GSK101 (black) at −60 and 60 mV. (FIG. 2G) There were no significant differences in currents among hTRPV4, hTRPV4-R269H and hTRPV4-¹²¹AAWAA¹²⁵ when activating with 5 μM LPC 18:1 (48±6%, 42±6% and 43±4%, respectively). Data were normalized to activation obtained with 1 μM GSK101. n=5-6 cells/condition. One-way ANOVA with Tukey's post-test was used for FIG. 2G.

FIG. 3A-FIG. 31. LPC activates TRPV4 directly via a C-terminal binding pocket. (FIG. 3A) Sequence alignment of part of the C-terminus comprising the TRP helix of rTRPV1, xTRPV4, rTRPV4 and hTRPV4. Note conservation of positive charge at position K710 for TRPV1, R742 for xTRPV4, and R746 for rTRPV4 and hTRPV4 (star). Identical residues, shared between TRPV1 and TRPV4, C-terminal of this key residue are bolded in black. Identical residues, conserved only in vertebrate TRPV4, C-terminal to R742/R746 are bolded and underlined. (FIG. 3B-FIG. 3E) Representative currents from excised inside-out membrane patches in HEK cells transfected with rTRPV4 (FIG. 3B) or rTRPV4-R746D (FIG. 3C) were obtained in the absence of agonist stimulation (light gray), in the presence of 5 μM LPC 18:1 (middle gray) or 1 μM GSK1016790A (GSK101, black). Traces shown were obtained at −60 and 60 mV. (FIG. 3D) There was a significant reduction of currents in rTRPV4-R746D transfected HEK cells when activated with 5 μM LPC 18:1 (average activation was 54±4% for WT-rTRPV4 and 15±3% for rTRPV4-R746D). Data were normalized to activation obtained with 1 μM GSK101. ***p<0.001 vs. rTRPV4, n=5 cells/group. (FIG. 3E) In vitro interaction assays show significantly reduced binding of LPC 18:1 to rTRPV4-R746D, relative to rTRPV4. ***p<0.001 vs. rTRPV4, n=3 assays/group. (FIG. 3F-FIG. 3G) Based on alignment and established TRPV4 crystal and cryo-EM structure, derived from Xenopus tropicalis TRPV4, these figures show our structural model that explains binding of LPC 18:1 to a series of positively charged AA750-772; with R742 is a postulated structural determinant of this binding. Left-hand rendering shows the TRPV4 tetramer (each subunit in different color) as it integrates into the plasma membrane, with the green subunit binding of LPC 18:1. Right-hand schematic shows binding of LPC 18:1 to the TRPV4 C-terminus at higher resolution. (FIG. 3H-FIG. 31) The Ca²⁺ signal-induced by 10 μM LPC 18:1 was drastically reduced in HEK cells transfected with rat or human TRPV4 mutations (R746C, R746G, R746D, K754G, R757G, R774G, and W776G). In contrast, 10 nM GSK101-induced Ca²⁺ signal was not significantly disrupted, except that we noticed a reduction with mutation W776G. **p<0.01 and ***p<0.001 vs. EGFP, #p<0.05 and ##p<0.01 vs. hTRPV4 or rTRPV4, n≥120 cells recorded/condition. Two-tailed t-test was used for FIG. 3D-FIG. 3E, and one-way ANOVA with Tukey's post-test for FIG. 3H-FIG. 31.

FIG. 4A-FIG. 4L. LPC elicits extracellular release of miR-146a from skin keratinocytes depending on TRPV4→pERK→Rab5a/Rab27a signaling. (FIG. 4A-FIG. 4B) LPC (10 μM) stimulation increased pERK expression in cultured mouse keratinocytes (KC, FIG. 4A) and human keratinocytes (KC, FIG. 4B) that was abolished by pretreatment with TRPV4-selective inhibitors GSK205 or HC067047 (both at 10 μM). *p<0.05 vs Veh. (0.2% DMSO). #p<0.05 and ##p<0.01 vs. LPC, n=4-6 cultures/group (5-7 pups/culture for mice, 2 subjects/culture for human). (FIG. 4C) I.d. injection of LPC (500 μg/50 μL) increased pERK expression in dissected dorsal neck skin that was reversed by i.p. pretreatment with TRPV4-selective inhibitor GSK205 (20 mg/kg). Elimination of the increase of pERK expression was also observed in skin from keratinocyte-Trpv4 cKO. *p<0.05 vs Veh. (normal saline), ##p<0.01 vs. LPC, n=7 mice/group. (FIG. 4D) I.d injection of LPC induced itch, which was significantly attenuated in mice i.d. pretreated with the MEK selective inhibitor U0126 (20 μg/50 μL). ***p<0.001 vs LPC, n=11 mice for LPC and 6 for U0126+ LPC. (FIG. 4E) Immunostaining detected increased p-MEK expression in dorsal neck skin 2 d after induction of the B-raf transgene in keratinocytes by treatment of K5cre::B-raf^{CA/+} mice with 4-OH tamoxifen (arrows: epidermis; blue: DAPI). (FIG. 4F) Western blot detected an increased p-ERK expression in dorsal neck skin 2 d after treatment of K5cre::B-raf^{CA/+} mice with 4-OH tamoxifen. **p<0.01 vs. Veh. (EtOH), n=4 mice for Veh. and n=7 for tamoxifen group. (FIG. 4G) Activation of B-raf by 4-OH tamoxifen in skin keratinocytes elicited a strong spontaneous scratching behavior on day 2. *p<0.05 vs. Veh. (EtOH), n=5 mice for Veh. and n=8 for tamoxifen group. (FIG. 4H) LPC stimulation (10 μM) led to extracellular release of miR-146a from cultured mouse and human keratinocytes (KC) that was completely eliminated by pretreatment with TRPV4 inhibitors GSK205 or HC067047 (both at 10 μM). *p<0.05 vs. Veh. (medium), ^{#}p<0.05 and ^{##}p<0.01 vs. LPC, n=3-4 cultures/treatment (5-7 pups/culture for mice, 2 subjects/culture for human). (FIG. 4I) LPC stimulation (10 μM) led to extracellular release of miR-146a from cultured mouse (FIG. 4J) and human (FIG. 4K) keratinocytes that was abolished by pretreatment with selective MEK inhibitor U0126 (10 μM). *p<0.05 and *p<0.01 vs. Veh. (medium), #p<0.05 vs. LPC, n=4 cultures/treatment (5-7 pups/culture for mice, 2 subjects/culture for human). (FIG. 4J) Quantification of extracellular release using a fluorescent enzymatic method shows that LPC (10 μM for 10 min)-induced a moderate but significant vesicular release from mKC, which was completely eliminated by inhibition of ERK with selective inhibitor U0126 (10 μM). **p<0.01 vs. Control and ^{##}p<0.01 U0126+ LPC vs. LPC. N=4-6 cultures (5-7 pups/culture). (FIG. 4K) Using the same set-up and quantification method as in FIG. 4J, we detected a significant decrease of LPC-elicited vesicular release from mKC treated with Rab27a or Rab5a siRNA (scrambled siRNA control set at ‘1’ for relative comparison). *p<0.05 vs. Scramble+LPC. N=4-6 cultures (5-7 pups/culture). (FIG. 4L) RT-qPCR assay detected a significant decrease of LPC-elicited (10 μM, 10 min) release of miR-146a from mKC by siRNA-mediated knockdown of Rab27a or Rab5a. *p<0.05 vs Scramble+LPC. N=4-5 cultures (5-7 pups/culture). One-way ANOVA with Tukey's post-test was used for FIG. 4A-FIG. 4C, FIG. 4G, FIG. 4H-FIG. 4L, and two-tailed t-test for FIG. 4D and FIG. 4F.

FIG. 5A-FIG. 5G. miR-146a elicits scratching behavior, which requires TRPV1, but not TRPA1, in sensory neurons. (FIG. 5A) I.d. injection of miR-146a induced dose-dependent scratching behavior, but miR-146a scramble (4 nmol) did not cause significant scratching behavior. *p<0.05 and ***p<0.001 vs. normal saline (NS), n=4-5 mice/group. (FIG. 5B) The mouse cheek model demonstrated that i.d. injection of miR-146a into the cheek elicited a robust scratching response (indicative of pruritus) but not a wiping response (indicative of pain). ***p<0.001 vs. NS, n=4-5 mice/group. (FIG. 5C) Elimination of TRPV1-expressing spinal nerve terminals with i.t. injection of resiniferatoxin (RTX, 200 ng/5 μL) significantly reduced miR-146a- or LPC-induced itch. *p<0.05 and **p<0.01 vs. Veh. (2% EtOH+2% Tween-80), n=4-5 mice/group except for 11 mice for i.t. Veh.+LPC. (FIG. 5D-FIG. 5E) miR-146a-induced or LPC-induced itch was significantly attenuated by i.p. (2 mg/kg) or i.t. (30 μg) injection of the TRPV1 inhibitor SB366791, or knockout of Trpv1. *p<0.05 and **p<0.01 vs. WT, n=4-5 mice/group for FIG. 5D and n=6-11 mice/group for FIG. 5E. (FIG. 5F-FIG. 5G) miR-146a-induced or LPC-induced itch was not significantly altered by i.p. (30 mg/kg) or i.t. (30 μg) injection of the TRPA1 inhibitor HC030031, or knockout of Trpa1. n=4-5 mice/group for FIG. 5F and n=5-11 mice/group for FIG. 5G. One-way ANOVA with Tukey's post-test was used for FIG. 5A, FIG. 5D-FIG. 5G, and two-tailed t-test for FIG. 5B-FIG. 5C.

FIG. 6A-FIG. 6G. miR-146a activates primary sensory neurons in a TRPV1-, but not TRPA1-, dependent manner. (FIG. 6A) miR-146a induced Ca²⁺ influx in cultured DRG sensory neurons in a dose-dependent manner (arrow: stimulation with miR-146a or scramble). n≥190 neurons recorded/concentration. (FIG. 6B) miR-146a (300 nM) induced Ca²⁺ influx that was significantly reduced by pretreatment with the TRPV1 inhibitor SB366791 (10 μM) and in neurons derived from Trpv1 KOs. In contrast, it was not significantly altered by inhibition of TRPA1 with HC030031 (10 μM) or in neurons derived from Trpa1 KOs. **p<0.01 vs. Scramble (300 nM) and ^{#}p<0.05 vs. miR-146a, n≥190 neurons recorded/concentration. (FIG. 6C) Representative Ca²⁺ imaging of GCaMP3-expressing DRG neurons in an ex-vivo preparation illustrates the increased Ca²⁺ signal in sensory neurons following stimulation with miR-146a (300 nM) and capsaicin (1 μM). (FIG. 6D) Representative Ca²⁺ traces of miR-146a(+)/capsaicin(−), miR-146a(+)/capsaicin(+), or miR-146a(−)/capsaicin(+) DRG neurons. (FIG. 6E) Representative Ca²⁺ traces of a population of sensory neurons responsive to miR-146a. (FIG. 6F) Of 1250 neurons recorded, 154 were responsive to miR-146a. 72.7% of miR-146a-responsive neurons were also capsaicin responsive. (FIG. 6G) Increased percentage of total DRG neurons responding to miR-146a (300 nM) was significantly reduced by inhibition of TRPV1 with SB366791 (10 μM), but not by inhibition of TRPA1 with HC030031 (10 μM). **p<0.01 vs. Scramble (300 nM) and ^{#}p<0.05 vs. miR-146a, n=4-9 DRG explants/group (1 explant per mouse). One-way ANOVA with Tukey's post-test was used for FIG. 6B and FIG. 6G.

FIG. 7A-FIG. 7H. LPC and miR-146a are elevated in mice or primary biliary cholangitis (PBC) patients with cholestatic itch and induce itch in nonhuman primates. (FIG. 7A) ANIT treatment increased LPC levels in both serum and skin. **p<0.01, and ***p<0.001 vs. WT control, two-tailed t test. N=5-8/group. (FIG. 7B) ANIT induced an increase of miR-146a in serum, which was attenuated in keratinocyte-Trpv4 cKO. *p<0.05 vs. WT control, and #p<0.05 vs K14-cre::Trpv4^{fl/fl} (tam), one-way ANOVA with Tukey's post test. N=8-11/group. (FIG. 7C) ANIT (25 mg/kg)-induced cholestatic itch was significantly attenuated in keratinocyte-Trpv4 cKO, *p<0.05, **p<0.01, and ***p<0.001 vs. WT control, ^{#}p<0.05 vs. K14-cre::Trpv4^{fl/fl} (tam), two-way ANOVA with Tukey's post test. N=6/group. (FIG. 7D) All detected LPC species, except for LPC 20:4, were significantly elevated in sera of PBC patients with itch (n=27) vs. PBC patients without itch (n=21), *p<0.05 and **p<0.01, two-tailed t-test. (FIG. 7E) When all detected LPC species from individual patients were aggregated, there was a significant linear correlation of total LPC concentration with the itch intensity ranging from 0 (no itch) to 10 (severe itch). Pearson's correlation coefficient: R=0.4314, p=0.0029. (FIG. 7F) A significant increase in abundance of miR-146a was detected in sera of PBC patients with itch (n=10) vs. PBC patients without itch (n=12), **p<0.01, two-tailed t-test. (FIG. 7G) There was a significant linear correlation of miR-146a level with the itch intensity ranging from 0 (no itch) to 10 (severe itch). Pearson's correlation coefficient: R=0.4536, p=0.034. (FIG. 7H) I.d. injection of LPC or miR-146a induced scratching behavior in rhesus monkeys in a dose dependent manner. I.d histamine was used as positive control to induce scratching behavior. ***p<0.001, ^{#}p<0.05, ^{##}p<0.01, 444p<0.05, and ^{$$$}p<0.001 vs. normal saline (NS), repeated measures using a linear mixed model (see more details in Statistical Analysis in Example 1). N=9 monkeys/group.

FIG. 8. Schematic diagram depicting the potential mechanism underlying cholestatic itch. Cholestatic liver disease is associated with significantly elevated systemic LPC, which directly activates TRPV4 expressed in skin keratinocytes. This in turn leads to extracellular release of miR-146a via MEK-ERK-Rab5a/Rab27a signaling pathways. miR-146a functions as a pruritogen by activating TRPV1-expressing pruriceptor sensory neurons that innervate the skin. Activation of TRPV1 by miR-146a induces the sensation of itch via central pathways.

FIG. 9A-FIG. 9D. Depletion of Trpv4 mRNA and protein in dorsal root ganglion (DRG) and trigeminal ganglion (TG) of Nav1.8-Cre::Trpv4^{fl/fl} mice. (FIG. 9A) qRT-PCR shows that Trpv4 mRNA was significantly reduced in Nav1.8-Cre::Trpv4^{fl/fl} mice. *p<0.05 vs WT, two-tailed t-test, n=4-5 mice/group. (FIG. 9B-FIG. 9D) Immunostaining with their respective, specific antibodies shows reduced TRPV4-expressing neurons (FIG. 9B), but unchanged TRPV1-expressing neurons (FIG. 9C) or TRPA1-expressing neurons (FIG. 9D), in Nav1.8-Cre::Trpv4^{fl/fl} mice. Arrows represent TRP-positive neurons. ***p<0.001 vs WT, two-tailed t-test, n=4 mice/group (>1200 total neurons counted/group).

FIG. 10A-FIG. 10B. Intrathecal (i.t.) injection of LPC induces mechanical pain via activation of TRPV4-expressing dorsal root ganglion (DRG) sensory neurons. (FIG. 10A) Inhibition of TRPV4 with its selective inhibitor GSK205 (10 μM) reduced LPC (10 μM)-induced Ca²⁺ signal in cultured DRG neurons. **p<0.01 vs. LPC, two-tailed t test. N≥250 cells recorded/treatment. (FIG. 10B) Single i.t. injection of LPC (15 μg/5 μL) induced long-lasting pain, as evidenced by reduced mechanical withdrawal thresholds. Pain behavior was attenuated in sensory neuron-Trpv4 cKO (Nav1.8-Cre::Trpv4^{fl/fl}). ^{#}p<0.05 and ^{###}p<0.001 vs. WT: Vehicle (normal saline), and *p<0.05 and **p<0.01 vs. WT: LPC, two-way ANOVA with Tukey's post test. N=7-8 mice/group.

FIG. 11A-FIG. 11C. Keratinocyte-TRPV3 is not involved in LPC-induced itch. LPC (10 μM)-induced Ca²⁺ signal in cultured mouse (FIG. 11A) or human (FIG. 11B) keratinocytes was not significantly influenced by inhibition of TRPV3 with selective inhibitor IPP. One-way ANOVA with Tukey's post test, n≥180 cells recorded/treatment. (FIG. 11C) i.d. injection of LPC (500 μg)-induced itch was not significantly attenuated in mice pre treated with TRPV3 inhibitor IPP (10 mg/kg, i.p.). Two-tailed t test. N=13 mice for LPC and 5 for IPP+LPC.

FIG. 12A-FIG. 12C. Lack of evidence for GPCR-signaling upstream of TRPV4 in Ca²⁺ influx. Ca²⁺ influx in cultured mouse (FIG. 12A) and human (FIG. 12B) keratinocytes (KC) and in HEK cells transfected with hTRPV4 (FIG. 12C), induced by LPC 18:1 (10 μM), was not significantly altered by Gα_{q} inhibitor BIM46187 (BIM), phospholipase C inhibitor U73122, or Gβγ inhibitor Gallein (all at 10 μM). One-way ANOVA with Tukey's post test. n≥140 cells recorded/treatment.

FIG. 13A-FIG. 13D. Electrophysiology findings of hTRPV4 with mutations at R746 reiterate its critical relevance for channel activation by LPC 18:1. (FIG. 13A-FIG. 13C) Representative currents for hTRPV4, hTRPV4-R746G, and hTRPV4-R746C channels: Currents were recorded in the absence of agonist (light grey), in the presence of 5 μM LPC 18:1 (medium grey) or in the presence of 1 μM GSK101 (black) at −60 and 60 mV. (FIG. 13D) There was a significant reduction of currents in hTRPV4-R746C or hTRPV4-R746G transfected HEK cells when activating with 5 μM LPC 18:1 (average activation was 48±6% for WT-hTRPV4, 11±4% for hTRPV4-R746C, and 13±4% for hTRPV4-R746G). Data were normalized to activation obtained with 1 μM GSK101. ***p<0.001 vs. hTRPV4, One-way ANOVA with Tukey's post test. N=5-10 cells/condition.

FIG. 14. Overlay of all the predicted conformer binding poses of LPC 18:1 (left, dark grey) and GSK 101 (right, light grey) relative to highlighted residues of xenopus TRPV4.

FIG. 15. ATP does not cause itch. I.d. injection of ATP at 10 nmol or 100 nmol did not elicit significant scratching behaviors when compared to control (normal saline). One-way ANOVA with Tukey's post test. N=5/group.

FIG. 16. LPC stimulation (10 μm, 15 min) did not increase the extracellular release of miR-let-7b, miR-125b-1, miR-16-5p, or miR-203 from cultured mouse keratinocytes (KC). **p<0.001 vs. Veh. (medium), two tailed t-test was used. N=3 cultures/group (5-7 pups/culture).

FIG. 17A-FIG. 17B. Elimination of TRPV1⁺ central nerve terminals in superficial layers of the spinal cord by i.t. injection of resiniferatoxin (RTX). (FIG. 17A) Immunolabeling of TRPV1 in spinal cord of Veh. (5% DMSO+S % Tween80)- or RTX (200 ng/5 μL)-treated mice. (FIG. 17B) Quantification of TRPV1 immuno-reactive nerve terminals in superficial layers of spinal cord dorsal horn. *p<0.05 vs. Veh., two tail t-test, n=4-5 mice/group.

FIG. 18A-FIG. 18B. Lack of evidence of direct activation of TRPV1 or TLRs signaling upstream of TRPV1 in response to miR-146a. (FIG. 18A) Mouse scratching behavior evoked by i.d. injection of miR-146a (4 nmol) was not significantly altered by knockout of Tlr7, i.p. treatment with TLR7/9 inhibitor E6446 or TLR2/6 inhibitor (GIT27) at 10 mg/kg. One-way ANOVA with Tukey's post test. N=4-7/group. (FIG. 18B) HEK293 cells transfected with rTRPV1 or co-transfected with rTRPV1 and rTLRs did not show a significant Ca²⁺ transient upon stimulation with miR-146a at 300 nM when compared to control (GFP-transfected). One-way ANOVA with Tukey's post test. N≥260 cells/group.

### DETAILED DESCRIPTION

Described herein are compositions and methods for the diagnosis and treatment of an itch-related disorder. The present disclosure is based, in part, on the discovery of a species of glycero-phospho-lipid, lysophosphatidylcholine (LPC), as being elevated in blood of cholestatic patients, higher in those with itch versus those without itch, and in the blood and skin of cholestatic mice. Further, inhibiting TRPV4 specifically in skin abrogates the itch. These findings support the following: (1) TRPV4 expressed by skin keratinocytes is a target for specific treatment in cholestatic itch, uremic itch, pruritic psoriasis, other chronic pruritic disorders, cancers, lymphomas, infectious diseases and treatment side-effects; (2) TRPV4 expressed by skin keratinocytes is a target for specific treatment in any other form of itch with elevated lysophosphatidylcholine; (3) detection and/or measurement of TRPV4 expression in skin may be used as a diagnostic tool in itch; (4) genomic sequencing of TRPV4 may be used as a diagnostic tool in itch; (5) detection and/or measurement of lysophosphatidylcholine may be used as a disease marker for pruritic disorders in blood or skin; (6) detection and/or measurement of microRNA-146a may be used as a disease marker for pruritic disorders in blood or skin; (7) TRPV4 may be used as a molecular target for the development of TRPV4-modulatory molecules by making specific use of the newly discovered C-terminal binding site for lysophosphatidylcholine (positions 746-776); and (8) as in (6), detection and/or measurement of microRNA-146a may be used as a disease marker for pruritic disorders in blood or skin, in particular, with the discovery of new activator molecules that are not lethal upon systemic application.

### 1. DEFINITIONS

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Administration” or “administering” refers to delivery of a compound or composition by any appropriate route to achieve the desired effect. Administration may include any convenient route of administration, whether systemically/peripherally or at the site of desired action, including but not limited to, oral (for example, by ingestion); topical (including for example, transdermal, intranasal, ocular, buccal, and sublingual); pulmonary; respiratory (for example, by inhalation or insufflation therapy using, for example, an aerosol, for example, through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. In certain embodiments, administration may be topical. “Co-administered” refers to simultaneous or sequential administration. A compound or composition may be administered before, concurrently with, or after administration of another compound or composition. One skilled in the art can select an appropriate dosage and route of administration depending on the patient, the particular disease, disorder, or condition being treated, the duration of the treatment, concurrent therapies, etc. In certain embodiments, a dosage is selected that balances the effectiveness with the potential side effects, considering the severity of the disease, disorder, or condition (for example, itch).

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Biomarker” refers to a naturally occurring biological molecule present in a subject at varying concentrations useful in predicting the risk or incidence of a disease or a condition. The biomarker may be a small molecule, polynucleotide such mRNA or miRNA or a gene, a polypeptide or protein, lipid, or carbohydrate. For example, the biomarker can be a protein present in higher or lower amounts in a subject at risk for a disease or disorder. The biomarker can include nucleic acids, ribonucleic acids, or a polypeptide used as an indicator or marker for disease in the subject. In some embodiments, the biomarker is a protein. A biomarker may also comprise any naturally or nonnaturally occurring polymorphism (for example, single-nucleotide polymorphism [SNP]) present in a subject that is useful in predicting the risk or incidence of a disease.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The regulatory elements may include, for example, a promoter, an enhancer, an initiation codon, a stop codon, or a polyadenylation signal. The coding sequence may be codon optimized.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, for example, to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (for example, from Analyse-it Software Ltd., Leeds, UK; StateCorp LP, College Station, TX; SAS Institute Inc., Cary, NC.). The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof. In some embodiments, the control is from a healthy subject.

“Effective amount” refers to a dosage of a compound or composition effective for eliciting a desired effect, commensurate with a reasonable benefit/risk ratio. This term as used herein may also refer to an amount effective at bringing about a desired in vivo effect in an animal, preferably, a human, such as reduction in itch.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. The regulatory elements may include, for example, a promoter, an enhancer, an initiation codon, a stop codon, or a polyadenylation signal.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Pharmaceutically acceptable” means suitable for use in a human or other mammal. The terms “pharmaceutically acceptable carriers” and “pharmaceutically acceptable excipients” are used interchangeably and refer to substances that are useful for the preparation of a pharmaceutically acceptable composition. In certain embodiments, pharmaceutically acceptable carriers are generally compatible with the other ingredients of the composition, not deleterious to the recipient, and/or neither biologically nor otherwise undesirable. The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. In some embodiments, a carrier includes a solution at neutral pH. In some embodiments, a carrier includes a salt. In some embodiments, a carrier includes a buffered solution.

“Sample” or “test sample” as used herein can mean any sample in which the presence and/or level of a biomarker is to be detected or determined or any sample from a subject in need of the compositions or methods as detailed herein. Samples may include liquids, solutions, emulsions, or suspensions. Samples may include a medical sample. Samples may include any biological fluid or tissue, such as cells, biopsies, lymph, blood, whole blood, fractions of blood such as plasma and serum, muscle, interstitial fluid, sweat, saliva, urine, mucous, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. A biological sample may be obtained directly from a subject (for example, by blood or tissue sampling) or from a third party (for example, received from an intermediary, such as a healthcare provider or lab technician). In some embodiments, the sample comprises skin. In some embodiments, the sample comprises skin keratinocytes. In some embodiments, the sample comprises blood.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. “Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be capable of directing the delivery or transfer of a polynucleotide sequence to target cells, where it can be replicated or expressed. A vector may contain an origin of replication, one or more regulatory elements, and/or one or more coding sequences. A vector may be a viral vector, bacteriophage, bacterial artificial chromosome, plasmid, cosmid, or yeast artificial chromosome. A vector may be a DNA or RNA vector. A vector may be a self-replicating extrachromosomal vector. Viral vectors include, but are not limited to, adenovirus vector, adeno-associated virus (AAV) vector, retrovirus vector, or lentivirus vector.

### 2. BIOMARKERS FOR ITCH-RELATED DISORDERS

The compositions and methods detailed herein may be used to diagnose and/or treat an itch-related disorder. Itch-related disorders include those that cause the desire to scratch. The itch-related disorder may comprise itch, also referred to as pruritus. Itch may be chronic or acute. The itch-related disorders may be associated with TRPV4, miR-146a, and/or lysophosphatidylcholine. Itch-related disorders may include dermatological disorders and systemic disorders.

Dermatological disorders include disorders of the dermis and/or epidermis. Dermatological disorders include, but are not limited to, photo-induced inflammation, pain in diseases involving skin pain, itch, cancer, autoimmune diseases, fibrotic diseases, other acneiform or inflammatory skin diseases, and pigmentation disorders. For example, dermatological disorders may include, but are not limited to, sunburn; photoallergic reaction; phototoxic reaction; phytophotodermatitis (Berloque dermatitis); acute and chronic actinic dermatitis; atopic dermatitis exacerbation; all subtypes of rosacea including trigeminal-pain associated rosacea; all lupus erythematosus subtypes (systemic, discoid, subacute); atopic dermatitis; actinic prurigo; prurigo nodularis; prurigo subacuta; prurigo pigmentosa; Lichen simplex (also called neurodermatitis); diabetic pruritus; uremic pruritus; pruritus induced by metabolic (liver) diseases; pruritus induced by malignancies like lymphoma; pruritus induced by polycythemia vera; pruritus induced by scabies; pruritus induced by bullous pemphigoid; pruritus induced by urticaria (especially but not exclusively actinic urticaria); pruritus induced by insect/arachnoid vector bite; pruritus induced by parasitosis; melanoma; non-melanoma skin cancer (BCC, SCC); actinic keratosis and other premalignant skin cancers; mycosis fungoides; Sezary syndrome; Xeroderma pigmentosum; Cockayne syndrome; all lupus erythematosus subtypes (systemic, discoid, subacute); dermatomyositis; erythema multiforme; lichen planus; fibrotic diseases induced by UV-exposure (Rhinophyma, chronic actinic dermatitis, actinic reticuloid, photoaging, hyalinosis cutis et mucosae; polymorph light eruption; Acne aestivalis; all porphyria subforms with implications on photo-induced skin changes (erythropoetic porphyria, erythropoetic protoporphyria, Porphyria variegate); photo-induced Herpes simplex infection (Herpes labialis); morbus Darier; disseminated superficial actinic porokeratosis; pityriasis rubra pilaris; Bloom syndrome; Rothmund-Thomson syndrome; Hartnup syndrome photoaging; wrinkles; photo-induced inflammation; pigmentation; and pigmentation disorders.

The itch-related disorder may comprise itch manifesting from an underlying health condition. Systemic itch-related disorders may include liver disorder, kidney disorder, cancer such as lymphoma, infection such as bacterial infection or viral infection, or medication side-effects. Medication side effects may include side effects from medication including chloroquine and/or antibiotics. The itch-related disorder may include cholestatic itch or pruritus resulting from liver disease, such as, for example, primary biliary cirrhosis, primary sclerosing cholangitis, obstructive choledocholithiasis, carcinoma of the bile duct, cholestasis, hepatitis C viral infection, and other forms of viral hepatitis.

Itch may be associated with or result from conditions including, but not limited to, rosacea, atopic dermatitis, actinic prurigo, prurigo nodularis, prurigo subacuta, prurigo pigmentosa, Lichen simplex (also called neurodermatitis), diabetic pruritus, and uremic pruritus. Itch or pruritus may be associated with or result from conditions including metabolic (liver) diseases, malignancies like lymphoma, polycythemia vera, scabies, bullous pemphigoid, urticaria (especially but not exclusively actinic urticaria), insect/arachnoid vector bite, and parasitosis. Itch-related disorders may include, for example, diabetic pruritus; uremic pruritus; pruritus induced by metabolic (liver) diseases; pruritus induced by malignancies like lymphoma; pruritus induced by polycythemia vera; pruritus induced by scabies; pruritus induced by bullous pemphigoid; pruritus induced by urticaria (especially but not exclusively actinic urticaria); pruritus induced by insect/arachnoid vector bite; pruritus induced by parasitosis; cholestatic itch; uremic itch; pruritic psoriasis; pruritus of urticaria; Morbus During; and combinations thereof. In some embodiments, the itch-related disorder is selected from cholestatic itch, uremic itch, pruritic psoriasis, and combinations thereof.

The itch may be itch of the skin. The skin may include the dermis and/or epidermis. In some embodiments, the skin does not have inflammation. Regions of the body that may suffer from itch include, for example, skin, mucous membranes, eyes such as the conjunctiva, mucous membrane of the nose, paranasal passage, mouth, tongue, pharynx, genital skin, genital non-skin, genital epithelia, anal skin and/or mucous membrane and/or epithelia, rectal skin and/or mucous membrane and/or epithelia, eczema skin, eczema mucous membrane and/or epithelia, or combinations thereof.

Biomarkers for the itch-related disorder may include TRPV4, miRNA-146a, lysophosphatidylcholine (LPC), and combinations thereof.

a. TRPV4

The biomarker for the itch-related disorder may include TRPV4. In some embodiments, the biomarker for the itch-related disorder includes the level of expression of the TRPV4 gene. TRPV4 is a Ca2+-permeable, nonselective cation channel. TRPV4 functions in the regulation of systemic osmotic pressure by the brain, in vascular function, in liver, intestinal, renal and bladder function, in skin barrier function and response of the skin to ultraviolet-B radiation, in growth and structural integrity of the skeleton, in function of joints, in airway and lung function, in retinal and inner ear function, and in pain. The TRPV4 ion channel is activated by osmotic, mechanical and chemical cues. It also responds to thermal changes (warmth). Channel activation can be sensitized by inflammation and injury. TRPV4 is expressed in both innervated epithelia and sensory neurons. Keratinocytes abundantly express TRPV4. As detailed herein, TRPV4 expressed in epidermal keratinocytes plays a role in itch. TRPV4 also plays a role in UV-induced inflammation and pain. The TRPV4 channel exerts its role as a master regulator of UVB-evoked skin inflammation and nociception through Ca++ influx into keratinocytes. The UVB-evoked, TRPV4-mediated Ca++ influx re-programs the keratinocyte to function in a pro-inflammatory and pro-algesic (pro-pain) manner, via TRPV4-dependent secretion of endothelin-1 (ET-1), which may lead to sensation of itch and skin pigmentation. TRPV4 may comprise a polypeptide having an amino acid sequence of SEQ ID NO: 1, encoded by a polynucleotide of SEQ ID NO: 2. TRPV4 may comprise a polypeptide having an amino acid sequence of SEQ ID NO: 17, encoded by a polynucleotide of SEQ ID NO: 18.

b. Lysophosphatidylcholine

The biomarker for the itch-related disorder may include lysophosphatidylcholine (LPC). In some embodiments, the biomarker for the itch-related disorder includes the level of LPC. LPC is a metabolic precursor to lysophosphatidic acid (LPA), and LPA is a bioactive phospholipid with diverse biological functions. Autotaxin (ATX) catalyzes the hydrolysis of LPC to LPA, and levels of ATX and LPA correlate with itch intensity in patients with cholestatic liver disease. LPCs are present as minor phospholipids in the cell membrane and in the blood plasma. Since LPCs are quickly metabolized by lysophospholipase and LPC-acyltransferase, they last only shortly in vivo. As detailed in the Examples, LPC is robustly pruritic in mice, and TRPV4 in skin keratinocytes is important for LPC-induced itch and itch in mice with cholestasis. LPC levels are elevated in sera of primary biliary cholangitis patients with itch and correlate with itch intensity. Moreover, LPC levels are increased in sera of cholestatic mice and elicite itch in nonhuman primates. As detailed herein, LPC was discovered as a novel cholestatic pruritogen that induces itch through epithelia-sensory neuron crosstalk. LPCs are a class of chemical compounds that are derived from phosphatidylcholines. LPCs may differ in length of carbon backbone and/or differ in the number of carbon-carbon double bonds. A general formula for LPCs is shown below:

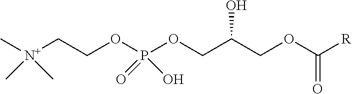

wherein R is a fatty acid chain. Examples of LPCs include, for example, LPC(18:1), LPC(14:0), LPC(16:0), LPC(16:1), LPC(17:0), LPC(18:0), LPC(18:2), LPC(20:3), LPC(20:4), LPC(24:0), LPC(26:0), LPC(26:1), LPC(28:0), and LPC(28:1), or any combination thereof. In some embodiments, the LPC is LPC(18:1).

c. miRNA-146a

The biomarker for the itch-related disorder may include microRNA-146a (miR-146a). In some embodiments, the biomarker for the itch-related disorder includes the level of miRNA-146a expression. In keratinocytes, TRPV4 activation by LPC induces extracellular release of miR-146a, which activates TRPV1+ sensory neurons to cause itch. miR-146a levels are elevated in sera of primary biliary cholangitis patients with itch and correlate with itch intensity. miR-146a levels are also increased in sera of cholestatic mice and elicit itch in nonhuman primates. As detailed in the Examples, scratching behavior and systemic concentration of miR-146a are dependent on keratinocyte-TRPV4. miR-146a may comprise a polynucleotide sequence of SEQ ID NO: 3.

### 3. LEVEL OF BIOMARKER

The level of the biomarker may be determined according to any suitable method known in the art. The level of LPC may be the level of the compound itself or a combination of various compound species thereof. The level of TRPV4 may be the level of expression of TRPV4. The level of miRNA-146a may be the level of expression of miRNA-146a. The level of expression of TRPV4 and/or miRNA-146a may be an RNA expression level. The level of expression of TRPV4 may be a protein level.

The amount or level of a biomarker (for example, a compound or small molecule) may be determined by any variety of techniques that are known in the art, such as, for example, by a method including chromatography and/or mass spectrometry, such as, for example, flow injection analysis-tandem mass spectrometry (FIA-MS/MS). Levels of LPC in a sample, such as serum or skin from a subject, may be determined by an enzymatic colorimetric method (Kishimoto et al. Clinical biochemistry 2002, 35, 411-416). Serum levels of LPC in samples from subjects may be determined by the AbsoluteIDQ™ p180 kit (Biocrates, Life Sciences AG, Innsbruck, Austria). Detection of the biomarker may include serial dilutions and/or internal standards. Different species of LPC may be detected, such as at least 2 species, as at least 3 species, at least 4 species, at least 5 species, at least 6 species, at least 7 species, at least 8 species, at least 9 species, at least 10 species, at least 11 species, at least 12 species, at least 13 species, at least 14 species, or at least 15 species of LPCs.

The amount or level of expression of a biomarker or biomolecule (for example, mRNA or protein) in a cell may be evaluated by any variety of techniques that are known in the art. The level of miRNA-146a may be the level of expression of the mRNA. The level of mRNA may be determined by microarray analysis, binding of a labelled probe complementary to the mRNA and detection of the labelled probe, PCR such as RT-PCR or RT-qPCR, or a combination thereof. The level of gene expression or protein expression (for example, TRPV4) may be evaluated at the protein or mRNA level using techniques including, but not limited to, Western blot, ELISA, Northern blot, real time PCR, immunofluorescence, FACS analysis, microarray analysis of the mRNA, binding of a labelled probe complementary to the mRNA and detection of the labelled probe, PCR such as RT-PCR or RT-qPCR, or a combination thereof. For example, the expression level of a protein may be evaluated by immunofluorescence by visualizing cells stained with a fluorescently-labeled protein-specific antibody, Western blot analysis of protein expression, and/or PCR such as RT-PCR or RT-qPCR of protein transcripts.

The amount or level of expression of a biomarker may be compared to a control. The comparison may be made to the amount or level of expression in a control cell, such as a non-disease cell or other normal or healthy cell. Alternatively, the control may include an average range of the amount or level of expression from a population of normal or healthy cells. Alternatively, a standard value developed by analyzing the results of a population of cells with known responses to therapies or agents may be used. Those skilled in the art will appreciate that any of a variety of controls may be used.

In some embodiments, the amount or level of expression of the biomarker is greater in a biological sample from a subject than in a control sample. In some embodiments, the presence of one or more biomarker(s) in a sample from a subject in an amount greater than a control is indicative of the itch-related disorder in the subject. In some embodiments, a subject is identified as having the itch-related disorder when the amount or level of expression of the biomarker is greater in a biological sample from the subject than in a control sample.

### 4. ANTI-PRURITIC THERAPY

An anti-pruritic therapy may be administered to a subject having an itch-related disorder as detailed herein. Anti-pruritic therapy includes anti-itch therapy. The anti-pruritic therapy may include one or more compounds and compositions. The anti-pruritic therapy may include a small molecule. The anti-pruritic therapy may comprise a polynucleotide, polypeptide, carbohydrate, lipid, or a combination thereof. The anti-pruritic therapy may comprise an antibody. The anti-pruritic therapy may comprise a biological molecule, including nucleic acid molecules, such as a polynucleotide having RNAi activity against, for example, TRPV4 or a substrate thereof. Anti-pruritic therapy includes current, emerging, and possible future therapies for itch and can be administered either by the subject or via a medical professional. Specific treatments depend on many factors, including the etiology of the itch, patient diagnosis, patients characteristics (for example, age, weight, health, etc.) and can be readily determined by one skilled in the art.

The anti-pruritic therapy may be selected from (a) topical treatments such as moisturizers, capsaicin, salicylic acid, emollients, topical corticosteroids, topical calcineurin inhibitors, antihistamines, menthol, local anesthetics, cannabinoids, immunomodulators, antihistamines (for example, Doxepin 5%) menthol, local anesthetics (for example, pramoxine 1%-2.5%, lidocaine patch 5%, eutectic mixture of lidocaine 2.5% and prilocaine 2.5%, 5% urea+3% polidocanol, etc.), cannabinoids (for example, creams containing N-palmitoylethanolamine), immunomodulators (TRPV1, TRPV4, etc.); (b) systemic treatments such as antihistamines, antidepressants (for example, SNRIs such as Mirtazapine 7.5-15 mg PO qd), SSRIs (for example, Paroxetime 10 mg-40 mg PO qd, Fluvoxamine 25 mg-150 mg PO qd, Sertraline 75 mg-100 mg PO qd), μ-opiod receptor agonists (for example, naltrexone 25 mg-50 mg PO qd), k-opiod receptor agonists (for example, butorphanol 1 mg-4 mg intransally qd, nalfurafine 2.5 μg-5 μg PO qd), neuroleptics (for example, gabapentin 100 mg-3600 mg PO qd, pregabalin 150 mg-300 mg PO qd), substance P antagonist (for example, aprepitant 80 mg PO qd), immunosuppressants (for example, cyclosporin 2.5-5 mg/kg PO qd, azathioprine 2.5 mg/kg PO qd), methylnaltrexone, NGX-4010, TS-022, Serine proteases/PAR2 antagonists, IL-31 antibody, IL-4-receptor antibody, IL-13 antibody, TSLP-antibody, IL-5 antibody; and (c) combinations thereof. In some embodiments, the anti-pruritic therapy comprises an immunomodulator.

In some embodiments, the anti-pruritic therapy comprises a TRPV4 inhibitor. In some embodiments, the immunomodulator comprises a TRPV4 inhibitor. TRPV4 inhibitors are described in, for example, WO2014/008477, WO2016/028325, and WO2017/177200, incorporated herein by reference.

A TRPV4 inhibitor can inhibit the biological function of TRPV4 (for example, inhibit cation channel activity, inhibit Ca++ permeation and/or availability). Other embodiments provide for a TRPV4 inhibitor that inhibits the expression of mRNA encoding TRPV4. Some embodiments provide a TRPV4 inhibitor that inhibits the translation of mRNA encoding TRPV4 to protein. A TRPV4 may be an allosteric modulator. Thus, a TRPV4 inhibitor may indirectly or directly bind and inhibit the activity of TRPV4 (for example, binding activity or enzymatic activity), reduce the expression of TRPV4, prevent expression of TRPV4, or inhibit the production of TRPV4 in a cell. Inhibit or inhibiting relates to any measurable reduction or attenuation of amounts or activity, for example, amounts or activity of TRPV4, such as those disclosed herein. “Amounts” and “levels” of protein or expression may be used herein interchangeably.

In some embodiments, a TRPV4 inhibitor can increase the amount of, or the biological activity of, a protein that can reduce the activity of TRPV4. Inhibitors capable of increasing the level of such a protein may include any inhibitor capable of increasing protein or mRNA levels or increasing the expression of the protein that inhibits TRPV4. In one embodiment, a TRPV4 inhibitor may comprise the protein itself. For example, a TRPV4 inhibitor may include exogenously expressed and isolated protein capable of being delivered to the cells. The protein may be delivered to cells by a variety of methods, including fusion to Tat or VP16 or via a delivery vehicle, such as a liposome, all of which allow delivery of protein-based inhibitors across the cellular membrane. Those of skill in the art will appreciate that other delivery mechanisms for proteins may be used. Alternatively, mRNA expression may be enhanced relative to control cells by contact with a TRPV4 inhibitor. For example, an inhibitor capable of increasing the level of a natively expressed protein that inhibits TRPV4 may include a gene expression activator or de-repressor. As another example, a TRPV4 inhibitor capable of decreasing the level of natively expressed TRPV4 protein may include a gene expression repressor. An inhibitor capable of increasing the level of a protein that inhibits TRPV4 may also include inhibitors that bind to directly or indirectly and increase the effective level of the protein, for example, by enhancing the binding or other activity of the protein. An inhibitor capable of decreasing the level of TRPV4 protein may also include compounds or compositions that bind to directly or indirectly and decrease the effective level of TRPV4 protein, for example, by inhibiting or reducing the binding or other activity of the TRPV4 protein.

A TRPV4 inhibitor may comprise a variety of compounds and compositions and agents. For example, a TRPV4 inhibitor may comprise a compound. A TRPV4 inhibitor may be a small molecule. A TRPV4 inhibitor may comprise a polynucleotide, polypeptide, carbohydrate, lipid, or a combination thereof. A TRPV4 inhibitor may comprise an antibody. A TRPV4 inhibitor may comprise an aptamer. A TRPV4 inhibitor may comprise a biological molecule, including nucleic acid molecules, such as a polynucleotide having RNAi activity against TRPV4 or a substrate thereof. In some embodiments, the nucleic acid molecules include RNAs, dsRNAs, miRNAs, siRNAs, nucleic acid aptamers, antisense nucleic acid molecules, and enzymatic nucleic acid molecules that comprise a sequence that is sufficient to allow for binding to an encoding nucleic acid sequence and inhibit activity thereof (i.e., are complementary to such encoding nucleic acid sequences). Suitably, an RNAi molecule comprises a sequence that is complementary to at least a portion of a target sequence such that the RNAi can hybridize to the target sequence under physiological or artificially defined (for example, reaction) conditions. In some embodiments an RNAi molecule comprises a sequence that is complementary such that the molecule can hybridize to a target sequence under moderate or high stringency conditions, which are well known and can be determined by one of skill in the art. In some embodiments an RNAi molecule has complete (100%) complementarity over its entire length to a target sequence. A variety of RNAi molecules are known in the art, and can include chemical modifications, such as modifications to the sugar-phosphate backbone or nucleobase that are known in the art. The modifications may be selected by one of skill in the art to alter activity, binding, immune response, or other properties. In some embodiments, the RNAi can comprise an siRNA having a length from about 18 to about 24 nucleotides, about 5 to about 50 nucleotides, about 5 to about 30 nucleotides, or about 10 to about 20 nucleotides.

In some embodiments, the inhibitory nucleic acid molecule can bind to a target nucleic acid sequence under stringent binding conditions. The terms “stringent conditions” or “stringent hybridization conditions” include reference to conditions under which a polynucleotide will hybridize to its target sequence, to a detectably greater degree than other sequences (for example, at least 2-fold over background). An example of stringent conditions includes those in which hybridization in 50% formamide, 1 M NaCl, 1% SDS at 37° C., and a wash in 0.1×SSC at 60 to 65° C. is performed. Amino acid and polynucleotide identity, homology and/or similarity can be determined using the ClustalW algorithm, MEGALIGN™ (Lasergene, WI). Given a target polynucleotide sequence, for example, a nucleic acid encoding TRPV4 or biological substrate thereof, an inhibitory nucleic acid molecule can be designed using motifs and targeted to a region that is anticipated to be effective for inhibitory activity, such as is known in the art.

In other embodiments, anti-pruritic therapy comprises an antibody that can specifically bind to a protein such as, for example, TRPV4 or a fragment thereof. Embodiments also provide for an antibody that inhibits TRPV4 through specific binding to a TRPV4 substrate molecule. The antibodies can be produced by any method known in the art, such as by immunization with a full-length protein such as TRPV4, or fragments thereof. The antibodies can be polyclonal or monoclonal, and/or may be recombinant antibodies. In embodiments, antibodies that are human antibodies can be prepared, for example, by immunization of transgenic animals capable of producing a human antibody (see, for example, International Patent Application Publication No. WO 93/12227). Monoclonal antibodies (mAbs) can be produced by a variety of techniques, including conventional monoclonal antibody methodology, for example, the standard somatic cell hybridization technique of Kohler and Milstein, and other techniques, for example, viral or oncogenic transformation of B-lymphocytes. Animal systems for preparing hybridomas include mouse. Hybridoma production in the mouse is very well established, and immunization protocols and techniques for isolation of immunized splenocytes for fusion are well known in the art. Fusion partners (for example, murine myeloma cells) and fusion procedures are also known.

Any suitable methods can be used to evaluate a candidate active compound or composition for inhibitory activity toward TRPV4. Such methods can include, for example, in vitro assays, in vitro cell-based assays, ex vivo assays, and in vivo methods. The methods can evaluate binding activity, or an activity downstream of the enzyme of interest. Ex vivo assays may involve treatment of cells with an inhibitor of the invention, followed by detection of changes in transcription levels of certain genes, such as TRPV4 through collection of cellular RNA, conversion to cDNA, and quantification by quantitative real time polymerase chain reaction (RT-QPCR). Additionally, the cell viability or inflammation may be determined after treatment with an inhibitor. Activity of TRPV4 may be analyzed with any suitable method known in the art, for example, with a patch-clamp technique and/or Ca2+ imaging, as detailed in the Examples.

The TRPV4 inhibitor may inhibit or reduce the activity of TRPV4 by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 95%, at least about 1.5-fold, at least about 2-fold, at least about 3-fold, at least about 4-fold, at least about 5-fold, at least about 6-fold, at least about 7-fold, at least about 8-fold, at least about 9-fold, or at least about 10-fold.

a. LPC Binding Site on TRPV4

Further detailed herein is the binding site of LPC on TRPV4, as discovered by the inventors. The LPC binding site is located at the C-terminal end or portion of TRPV4. The LPC binding site is located C-terminally in the TRP-helix. The LPC binding site may comprise at least one amino acid in the motif comprising K742-W772 of TRPV4 from Xenopus, which corresponds to mammalian TRPV4 amino acids K746-W776. The LPC binding site may comprise at least one amino acid in the motif comprising K750-W772 of TRPV4 from Xenopus, which corresponds to mammalian TRPV4 amino acids K754-W776. The LPC binding site may comprise at least one amino acid in the motif comprising K750-W772 and R742 of TRPV4 from Xenopus, which corresponds to mammalian TRPV4 amino acids K754-W776 and R746. The LPC binding site may include at least one amino acid selected from K754, R757, R774, and W776 (of rTRPV4 or an amino acid corresponding thereto). Further provided herein is a compound that binds to the LPC binding site of TRPV4, as well as methods for screening compounds that bind to the LPC binding site of TRPV4, as detailed further below. The compound that binds to the LPC binding site of TRPV4 may modulate the activity of TRPV4, for example, it may inhibit or increase the activity of TRPV4.

The TRPV4 inhibitor may inhibit or reduce the activity of TRPV4 by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 95%, at least about 1.5-fold, at least about 2-fold, at least about 3-fold, at least about 4-fold, at least about 5-fold, at least about 6-fold, at least about 7-fold, at least about 8-fold, at least about 9-fold, or at least about 10-fold. The compound that binds to the LPC binding site of TRPV4 may increase the activity of TRPV4 by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 95%, at least about 1.5-fold, at least about 2-fold, at least about 3-fold, at least about 4-fold, at least about 5-fold, at least about 6-fold, at least about 7-fold, at least about 8-fold, at least about 9-fold, or at least about 10-fold.

b. Pharmaceutical Compositions

Further provided herein are pharmaceutical compositions comprising the anti-pruritic therapy. An anti-pruritic therapy as detailed herein may be formulated into pharmaceutical compositions in accordance with standard techniques well known to those skilled in the pharmaceutical art. The compounds (such as a TRPV4 inhibitor) may be administered in the form of compounds per se, or as pharmaceutical compositions comprising a compound. In some embodiments, the compound (for example, TRPV4 inhibitor) may be administered prior to, concurrently with, or after the one or more other therapeutic agents. In some embodiments, the anti-pruritic therapy comprises a compound, drug, etc. (for example, a TRPV4 inhibitor) are in the form of a pharmaceutical composition comprising, consisting or, or consisting essentially of the compound (for example, a TRPV4 inhibitor) and a pharmaceutically acceptable carrier and/or excipient. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid.

c. Administration

An anti-pruritic therapy, or a pharmaceutical composition comprising the same, may be administered or delivered to a cell. An anti-pruritic therapy, or a pharmaceutical composition comprising the same, may be administered to a subject. Such compositions can be administered in dosages and by techniques well known to those skilled in the medical arts taking into consideration such factors as the age, sex, weight, and condition of the particular subject, and the route of administration. The anti-pruritic therapy, or a pharmaceutical composition comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the anti-pruritic therapy, or a pharmaceutical composition comprising the same, is administered to a subject intramuscularly, intravenously, or a combination thereof. Any of the delivery methods and/or routes of administration detailed herein can be utilized with a myriad of cell types. The cell may be a stem cell such as a human stem cell. In some embodiments, the cell is a skin cell. The cell may be an epithelial cell. The cell may be a dermal cell. The cell may be an epidermal cell. In some embodiments, the cell is a keratinocyte.

In certain embodiments, compositions are formulated for topical administration. For compositions suitable for topical administration, the composition may be combined with one or more carriers and used in the form of cosmetic formulations. Formulations may include a foam, cream, gel, lotion, ointment, or solution. For example, a TRPV4 inhibitor may be suitably dissolved in the alcohol of skin disinfectant gel or in lotions, creams, or other formulations. In certain embodiments, a TRPV4 inhibitor may be included in or added to a cosmetic formulation. In certain embodiments, a TRPV4 inhibitor may be included in or added to sun protection topical formulations.

For oral therapeutic administration, the composition may be combined with one or more carriers and used in the form of ingestible tablets, buccal tablets, troches, capsules, elixirs, suspensions, syrups, wafers, chewing gums, foods, and the like. The percentage of the compositions and preparations may, of course, be varied and may conveniently be between about 0.1 to about 100% of the weight of a given unit dosage form. The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. The above listing is merely representative and one skilled in the art could envision other binders, excipients, sweetening agents and the like. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like.

### 5. METHODS

a. Methods Of Treating A Subject Having An Itch-Related Disorder

Provided herein are methods of treating a subject having an itch-related disorder. The methods may include determining the level of a biomarker in a biological sample from the subject, wherein the biomarker is selected from TRPV4 expression, lysophosphatidylcholine, and miRNA-146a expression, or a combination thereof, and administering an anti-pruritic therapy to treat the subject identified as having the itch-related disorder, wherein the subject is identified as having the itch-related disorder when the level of the biomarker is greater in the biological sample than in a control sample.

Further provided herein are methods of diagnosing and treating a subject having an itch-related disorder. The methods may include determining the level of a biomarker in a biological sample from the subject, wherein the biomarker is selected from TRPV4 expression, lysophosphatidylcholine, and miRNA-146a expression, or a combination thereof, and administering an anti-pruritic therapy to treat the subject identified as having the itch-related disorder, wherein the subject is identified as having the itch-related disorder when the level of the biomarker is greater in the biological sample than in a control sample.

In some embodiments, the biomarker is the level of TRPV4 expression. In some embodiments, the biomarker is the level of lysophosphatidylcholine. In some embodiments, the biomarker is the level of miRNA-146a expression.

b. Methods Of Treating An Itch-Related Disorder

Provided herein are methods of treating an itch-related disorder in a subject. The methods may include (a) determining the level of a biomarker in a biological sample from the subject, wherein the biomarker is selected from TRPV4 expression, lysophosphatidylcholine, and miRNA-146a expression, or a combination thereof, and wherein the level of the biomarker is greater in the biological sample than in a control sample; (b) diagnosing the subject as having an itch-related disorder based on the level of the biomarker determined in step (a); and (c) administering an anti-pruritic therapy to the subject diagnosed as having an itch-related disorder in step (b).

Further provided herein are methods of identifying and treating an itch-related disorder in a subject. The methods may include (a) determining the level of a biomarker in a biological sample from the subject, wherein the biomarker is selected from TRPV4 expression, lysophosphatidylcholine, and miRNA-146a expression, or a combination thereof, and wherein the level of the biomarker is greater in the biological sample than in a control sample; (b) diagnosing the subject as having an itch-related disorder based on the level of the biomarker determined in step (a); and (c) administering an anti-pruritic therapy to the subject diagnosed as having an itch-related disorder in step (b).

In some embodiments, the biomarker is the level of TRPV4 expression. In some embodiments, the biomarker is the level of lysophosphatidylcholine. In some embodiments, the biomarker is the level of miRNA-146a expression.

c. Methods of Diagnosing an Itch-Related Disorder in a Subject

Provided herein are methods of diagnosing or identifying an itch-related disorder in a subject. The methods may include determining the level of a biomarker in a biological sample from the subject, wherein the biomarker is selected from TRPV4 expression, lysophosphatidylcholine, and miRNA-146a expression, or a combination thereof; and diagnosing the subject as having an itch-related disorder when the level of the biomarker is greater in the biological sample than in a control sample. In some embodiments, the methods further include administering an anti-pruritic therapy to the subject diagnosed as having the itch-related disorder.

In some embodiments, the biomarker is the level of TRPV4 expression. In some embodiments, the biomarker is the level of lysophosphatidylcholine. In some embodiments, the biomarker is the level of miRNA-146a expression.

d. Methods of Identifying an Itch-Related Disorder in a Subject

Provided herein are methods of identifying an itch-related disorder in a subject. The methods may include (i) obtaining a biological sample from the subject; (ii) identifying the presence of a biomarker in the subject, the biomarker selected from the group consisting of TRPV4, miRNA-146a, lysophosphatidylcholine, and combinations thereof; (iii) quantifying the expression level of the biological sample, in which the presence of one or more of the biomarkers in an amount greater than the control is indicative of the itch-related disorder comprising itch; and (iv) administering to the subject an appropriate anti-pruritictherapy if the level of biomarker is greater in the biological sample than in a control sample.

In some embodiments, the biomarker is the level of TRPV4 expression. In some embodiments, the biomarker is the level of lysophosphatidylcholine. In some embodiments, the biomarker is the level of miRNA-146a expression.

e. Methods of Screening for a Compound that Modulates TRPV4

Provided herein is a method of screening for a compound that modulates TRPV4. The method may include testing a plurality of compounds for binding to wild-type TRPV4 to determine from the plurality of compounds a subset of compounds that bind wild-type TRPV4; and testing the subset of compounds that bind wild-type TRPV4 for binding to at least one mutant TRPV4, wherein the mutant TRPV4 comprises a mutation of at least one amino acid in the motif corresponding to R746-W776 of mammalian TRPV4 to determine from the subset of compounds a compound that binds wild-type TRPV4 but not the mutant TRPV4. The TRPV4 inhibitor may comprise a variety of compounds and compositions and agents. For example, a TRPV4 inhibitor may comprise a compound. A TRPV4 inhibitor may be a small molecule. A TRPV4 inhibitor may comprise polynucleotide, polypeptide, carbohydrate, lipid, or combination thereof.

In some embodiments, the mutant TRPV4 comprises a mutation of at least one amino acid selected from or in the motif corresponding to R746-W776 of mammalian TRPV4. In some embodiments, the mutant TRPV4 comprises a mutation of at least one amino acid selected from or in the motif corresponding to K754-W776 and R746 of mammalian TRPV4. In some embodiments, the mutant TRPV4 comprises a mutation of at least one amino acid selected from or in the motif corresponding to K754-W776 of mammalian TRPV4. In some embodiments, the mutant TRPV4 comprises a mutation of at least one amino acid selected from or in the motif comprising K754, R757, R774, and W776 of mammalian TRPV4. In some embodiments, at least one amino acid in the motif corresponding to K754-W776 of mammalian TRPV4 is mutated to an alanine. In some embodiments, at least one amino acid in the motif corresponding to K754-W776 of mammalian TRPV4 is mutated to a glycine. In some embodiments, at least one amino acid in the motif corresponding to K746-W776 of mammalian TRPV4 is mutated to an alanine. In some embodiments, at least one amino acid in the motif corresponding to K746-W776 of mammalian TRPV4 is mutated to a glycine. In some embodiments, at least one amino acid selected from K754, R757, R774, and W776 (of rTRPV4 or an amino acid corresponding thereto) is mutated, for example, to alanine or glycine. R757 and/or R774 may be mutated to lysine. K754 may be mutated to arginine. In some embodiments, the mutant TRPV4 has activity as an ion channel. In some embodiments, the mutant TRPV4 has activity as an ion channel that is at least 50% of the activity of wild-type TRPV4. In some embodiments, the method further comprises determining the effect of the compound that binds wild-type TRPV4 but not the mutant TRPV4 on the activity of wild-type TRPV4. The activity of TRPV4 may be analyzed with any suitable method known in the art, for example, with a patch-clamp technique and/or Ca2+ imaging, as detailed in the Examples. Modulators may include inhibitors and activators. In some embodiments, the compound that binds wild-type TRPV4 but not the mutant TRPV4 inhibits the activity of wild-type TRPV4. In some embodiments, the compound that binds wild-type TRPV4 but not the mutant TRPV4 increases the activity of wild-type TRPV4. In some embodiments, the compound that binds wild-type TRPV4 but not the mutant TRPV4 inhibits or reduces the binding of LCP to wild-type TRPV4. In some embodiments, the method further includes administering to a subject in need thereof, such as a subject having an itch-related disorder, the compound that binds wild-type TRPV4 but not the mutant TRPV4, in particular, wherein the compound inhibits wild-type TRPV4. The subject having an itch-related disorder may thereby be treated.

### 6. Examples

### Example 1

### Materials and Methods

Study design. The main objective was to determine whether LPC contributes to cholestatic itch via skin keratinocyte-sensory neuron crosstalk. The research subjects and units of investigation were cultured skin keratinocytes and DRG neurons from mouse and/or human, mouse or human sera, mouse DRG, spinal cord and skin tissues, live mice, or nonhuman primates in controlled laboratory experiments. Sample sizes for in vivo and in vitro assays were determined based on our experience with the experimental models, potential biological variables, and previous literature (Kremer et al. Gastroenterology 2010, 139, 1008-1018; Chen et al. J. Biol. Chem. 2016, 291, 10252-10262; Han et al. Neuron 2018, 99, 449-463.e446; Chen et al. Pain 2014, 155, 2662-2672; Lee et al. Sci. Rep. 2015, 5, 11676). Appropriate statistical analyses were performed to determine the differences between treatments or groups (see details in ‘Statistical Analysis’). Animals were randomly assigned into experimental groups. For behavior test and quantification, the investigators were blinded to treatments and group assignments.

Animals. Wild-type (WT) C57bl/6j mice were purchased from the Jackson Laboratory. Trpv4 knockout (KO) mice were generated in our laboratory as previously described (Liedtke et al. Proc. Natl. Acad. Sci. U.S.A. 2003, 100 Suppl 2, 14531-14536). Trpv1 KO (Stock No: 003770), Trpa1 KO (Stock No: 006401), and TIr7 KO (Stock No: 008380) mice, originally obtained from the Jackson Laboratory, were provided by Dr. Ru-Rong Ji at Duke University. Pirt-GCaMP3 mice (Kim et al. Neuron 2014, 81, 873-887), originally generated by Dr. Xinzhong Dong at Johns Hopkins University, were provided by Dr. Andrea Nackley at Duke University. Pirt-GCaMP3 mice express the calcium indicator, GCaMP3, in >96% of primary sensory neurons in the dorsal root ganglion (DRG) and trigeminal ganglion (TG).

Keratinocyte-specific, tamoxifen (tam)-inducible Trpv4 knockout mice were used as previously described (Chen et al. J. Biol. Chem. 2016, 291, 10252-10262; Chen et al. Pain 2014, 155, 2662-2672; Moore et al. Proc. Natl. Acad. Sci. U.S.A. 2013, 110, E3225-3234). In brief, the Trpv4 genomic locus was engineered so that loxP sites surrounded exon 13, which encodes TM5-6. This mutation was propagated in mice that were crossed to K14-Cre-ER^{tam} mice, so that K14-Cre-ER^{tam}::Trpv4^{lox/lox} mice could be induced by tamoxifen (tam) administration via oral gavage for five consecutive days at 5 mg/day in 0.25 mL corn oil at 2-2.5 months of age, plus a booster 2 weeks after the last application. Control animals received the same volume of corn oil. Efficiency of targeting was verified by quantitative real-time PCR and immunohistochemistry for Trpv4 expression in skin at gene and protein levels, respectively (Moore et al. Proc. Natl. Acad. Sci. U.S.A. 2013, 110, E3225-3234).

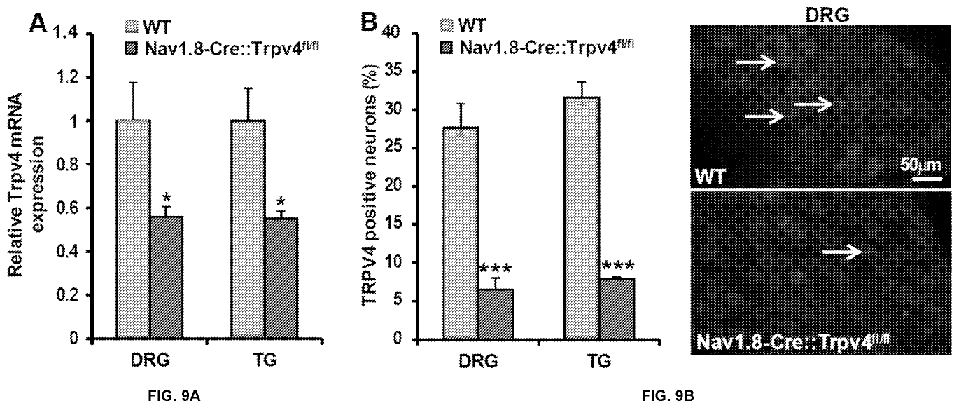

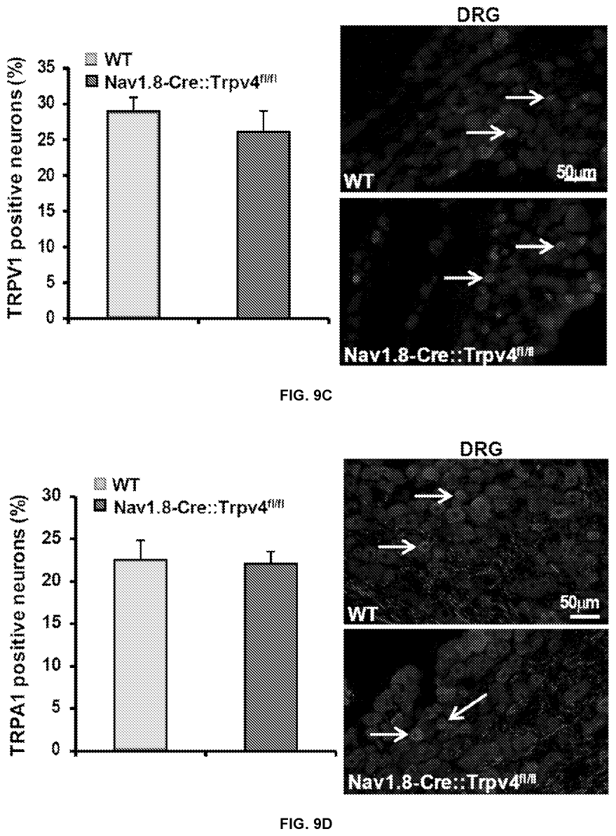

We also generated mice with deletion for Trpv4 in primary sensory neurons via Cre-loxP-mediated recombination by mating mice carrying Trpv4 (Trpv4^{fl/fl}) with a mouse line expressing Cre recombinase under control of the Nav1.8 promoter (Nav1.8-Cre). The Cre mice enable gene recombination commencing at birth selectively in sensory neurons expressing the sodium channel Nav1.8, without affecting gene expression in the spinal cord, brain, or any other organ in the body (Agarwal et al. Genesis 2004, 38, 122-129). Efficiency of targeting was verified by quantitative real-time PCR and immunohistochemistry for Trpv4 expression in both DRGs and TGs at gene and protein levels, respectively (FIG. 9A-FIG. 9D).

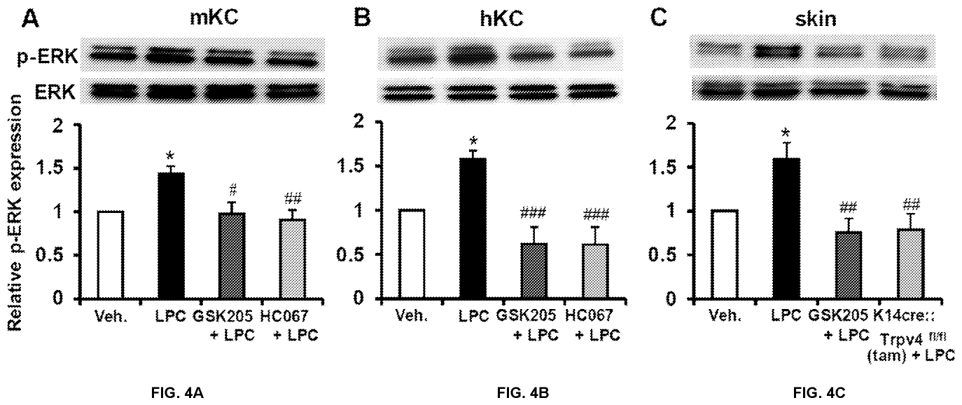

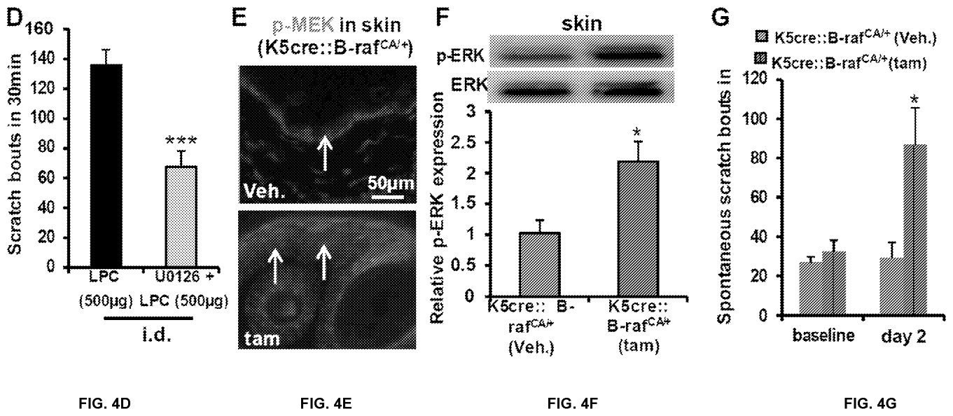

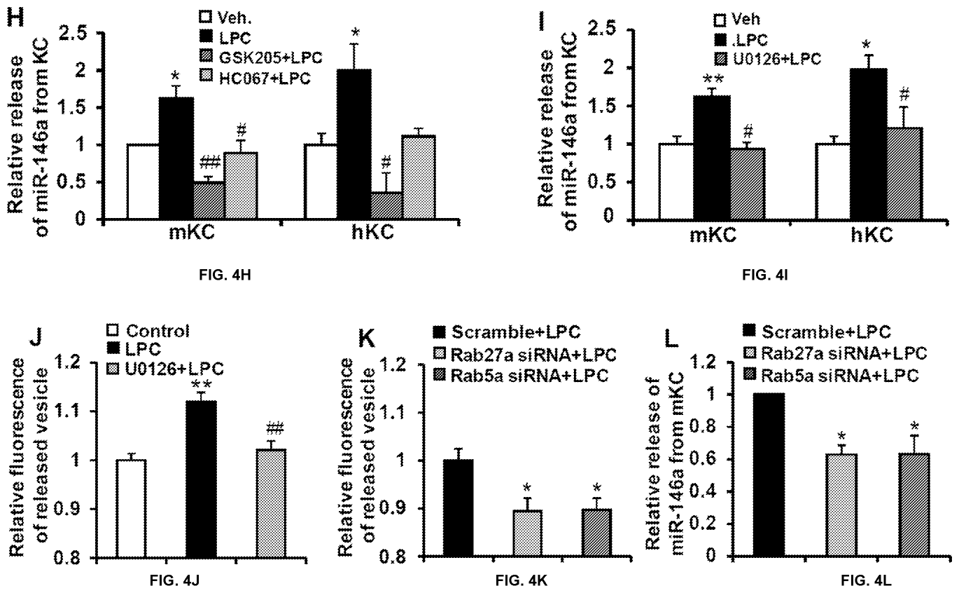

We also generated mice with inducible expression of constitutively active B-raf (V600E) in keratinocytes by crossing mice with a floxed allele for B-raf (B-raf^{CA/+} mice) (Dankort et al. Nat. Genet. 2009, 41, 544-552) with K5-cre-ER^{tam} mice (Keymeulen et al. Nature 2011, 479, 189-193 2011). The generated K5-cre-ER^{tam}::BrafCN⁺ mice were shaved at the dorsal back and topically treated with 4-hydroxy tamoxifen (100 μL of 20 mg/mL) in ethanol (EtOH) for 2 consecutive days. Control animals received the same volume of EtOH. Increased levels of p-MEK and p-ERK, downstream targets of B-raf, were detected in skin after treatment with 4-hydroxy tamoxifen, as verified by immunohistochemistry or Western blot (FIG. 4E-FIG. 4F).

Mice were housed in climate-controlled rooms on a 12/12-h light/dark cycle with water and a standardized rodent diet available ad libitum. All animal protocols were approved by the Duke University Institutional Animal Care and Use Committee (IACUC) in compliance with National Institutes of Health (NIH) guidelines.

All of these mouse lines have C57bl/6 background and were PCR-genotyped before use. Only male mice (2-3 months old) were used for in vivo behavioral assays.

In addition, adult male and female rhesus monkeys (Macaca mulatta, 11-18 years, 7.2-13.9 kg), were used for scratching behavior study. Monkeys were individually housed in species-specific and climate-controlled rooms on a 12/12-h light/dark cycle. Their daily diet consisted of approximately 22-30 biscuits (Purina Monkey Chow; Ralston Purina Co., St. Louis, MO), fresh fruit, and water ad libitum. Monkeys were kept at an indoor facility accredited by the Association for Assessment and Accreditation of Laboratory Animal Care International (Frederick, MD, USA). All animal care and experimental procedures were conducted in accordance with the Guide for the Care and Use of Laboratory Animals as adopted and promulgated by NIH and approved by the IACUC of Wake Forest University. This study is reported in accordance with the ARRIVE guidelines for reporting experiments involving animals.

Human subjects. Primary biliary cholangitis (PBC) patients were recruited at two clinical sites: Liver and Internal Medicine Unit of the Warsaw Medical University, Poland, and Department of Medicine 1, Gastroenterology, Hepatology, Pneumology and Endocrinology of the University Hospital of Erlangen, Germany. The diagnosis of PBC was made according to the European Association for the Study of Liver Disease criteria (Hirschfield et al. J. Hepatol. 2017, 67, 145-172). Itch intensity was quantified at the time-point of blood drawing using a visual analogue scale ranging from 0 to 10 (0-3: no/mild itch, 3-6: moderate itch, 6-10: severe/worst imaginable itch; Jacoby et al. Gut 2005, 54, 1622-1629; Raszeja-Wyszomirska et al. Clin. Res. Hepatol. Gastroenterol. 2016, 40, 471-479). The serum supernatant was aliquoted and frozen until measurements were performed. Patients' samples were used in anonymized manner for our study without any recourse to proprietary health information. The study population in Poland consisted of 27 women and 2 men, aged 36-75 years (average 55), and 10 women without itch, 9 women and 1 man with moderate itch, and 8 women and 1 man with severe itch. For study population in Germany, all patients were women, aged 29-64 years (average 51), and 11 patients without itch, 2 with moderate itch, and 6 with severe itch. Study protocols in Poland and Germany were approved by the local medical institutional review boards, and all subjects provided written consent for their samples to be used.

Chemicals and antibodies. LPA 18:1, egg-LPC (LPC, mixture of LPC species 14:0, 16:0, 16:1, 18:0, 18:1, and 18:2), LPC 14:0, LPC 16:0, LPC 18:0, and LPC 18:1 were purchased from Avanti Polar Lipids (Alabaster, AL). GSK1016790A, SB366791, HC030031, capsaicin, histamine, HC067047, PF8380, U73122, resiniferatoxin, U0126, tamoxifen, 4-hydroxy tamoxifen, α-naphthyl isothiocyanate (ANIT), isopentenyl pyrophosphate (IPP), glycerophosphorylcholine phosphodiesterase, choline oxidase, peroxidase, 3-(N-Ethyl-3-methylanilino)-2-hydroxypropanesulfonic acid sodium salt (TOOS), 4-aminoantipyrine, and adenosine triphosphate (ATP) were purchased from Sigma (St. Louis, MO). E6446 was purchased from Bocsci INC (Shirley, NY), BIM46187 was purchased from Aobious (Gloucester, MA), Gallein and GIT27 was purchased from Tocris Bioscience (Minneapolis, MN), and lysophospholipase was from Sekisui Diagnostics (Burlington, MA). GSK205 was synthesized by the Small Molecule Synthesis Facility at Duke University (Kanju et al. Sci. Rep. 2016, 6, 26894). miR-146a-5p mimic (miR-146a) and miR-146a-5p negative control (scramble) were obtained from Thermo Fisher (Waltham, MA). LPA, LPCs, histamine and ATP were dissolved in sterile normal saline (NS) and freshly made for use. miR-146a mimic and scramble were dissolved in nuclease-free water and freshly prepared before use. All other chemicals were dissolved in DMSO and further diluted until use.

Rabbit polyclonal anti-phospho-ERK, monoclonal anti-ERK, and polyclonal anti-phospho-MEK were obtained from Cell Signaling Technology (Danvers, MA). Rabbit polyclonal anti-TRPV1 was obtained from Neuromics (Edina, MN), polyclonal anti-TRPA1 from Novus Biologicals (Centennial, CO), and polyclonal anti-TRPV4 from Abcam (Cambridge, MA) from Sigma (St. Louis, MO). 4′,6-diamidino-2-phenylindole (DAPI) was obtained from Sigma (St. Louis, MO).

Behavioral assessment. Mice were shaved at the dorsal neck 1 d before experiments. Mice were allowed to acclimate to a Plexiglas chamber for at least 30 min before testing and received intradermal (i.d.) injection of 50 μL of LPA 18:1, LPC, LPC 14:0, LPC 16:0, LPC 18:0, LPC 18:1, miR-146a mimic, miR-146a scramble, ATP or vehicles through a 30-gauge needle (Becton Dickinson, Franklin Lakes, NJ) into the neck skin. After injections, mice were immediately placed back to the chamber, and the scratching behavior was recorded by a Panasonic video camera for 30 min. Hind limb scratching behavior directed toward the injected area at the nape of neck was observed. One scratch is defined as a lifting of the hind limb toward the injection site and then a replacing of the limb back to the floor, regardless of how many scratching strokes take place between those two movements. Behavioral analysis was conducted by observers blinded to genotype.

To investigate the effects of the selective inhibitors: GSK205 or HC067047 for TRPV4, SB366791 for TRPV1, HC030031 for TRPA1, PF8380 for autotaxin, U0126 for MEK, IPP for TRPV3, GIT for TLR2/6, and E6446 for TLR7/9, on LPA-, LPC- or miR-146α-induced scratching behaviors, mice received an intraperitoneal (i.p.) injection of 0.25 mL or an intrathecal (i.t., see approach below) injection of 5 μL of chemical solutions 15 min before pruritogen injections. Control animals received the same volume of vehicles.

To test whether LPC induces scratching behavior at the spinal cord level directly, a 30-gauge needle attached to 10 μL micro-syringe (Hamilton Co., Reno, NV) was inserted between L4 and L5 segments and tail flick upon i.t injection was considered a control for targeted delivery. LPC was injected into the subarachnoid space with a total volume of 5 μL at a constant rate of 10 μL/min. Scratching behavior was recorded for 30 min and behavioral analysis was performed as described above.

To examine whether i.d. injection of LPC induces pain-like behavior in mice, a mouse cheek model was used to differentiate itch from pain (Shimada et al. Pain 2008, 139, 681-687). In brief, 10 μL of LPC or miR-146a was administrated into the mouse cheek. Wiping (pain) and scratching (itch) behaviors were videoed for 30 min using a Panasonic camera. A bout of wiping was defined as a continuous wiping movement with a forepaw directing at the area of the injection area and a bout of scratching was defined as above.

To examine whether TRPV1-expressing sensory neurons contribute to LPC- or miR-146α-induced itch, we ablated the central terminals of TRPV1-expressing neurons by i.t. injection of 200 ng resiniferatoxin (RTX) into the L4/L5 subarachnoid space (Mishra et al. Mol. Cell. Neurosci. 2010, 43, 157-163). Lumbar puncture was made with a 30G-needle and drugs at 5 μL of volume were delivered. Scratching behavior assay was started approximately 2 weeks after resiniferatoxin treatment.

Scratching behaviors in monkeys were performed as previously described (Lee et al. Sci. Rep. 2015, 5, 11676). In brief, monkeys were seated in primate chairs and both lateral sides of upper part (i.e., the skin area over the vastus lateralis muscle) of their hindlimbs were shaved 1 d before experiments. The monkey's hindlimb was held by another experimenter during the injection. A 30G-needle connected with a 50 μL microsyringe (Hamilton Co., Reno, NV) was placed almost flat against skin, bevel up; and then was inserted ⅛ inch into skin. 20 μL of histamine, LPC or miR-146a solution was slowly injected and was watched for a wheal to appear. Once the injection was completed, monkeys immediately returned to his/her home cage and their potential site-specific scratching activity was recorded for 15 min after injection. A scratch was defined as one brief scraping contact of the forepaw or hind paw on the skin surface area. Scratching activities were scored by individuals who were blinded to dosing conditions. Before collecting data, monkeys had been habituated with the injection procedure and experimenter for several times. It is noted that the tested chemicals were i.d. administered to the same subjects with at least one-week interval, starting vehicle first and then chemicals in randomized doses. Based on our prior experience, we have not observed tolerance to elicited scratching with this schedule for a well-known pruritogen histamine. In addition, this repeated injection schedule with tested chemicals did not result in any skin lesion. There was no significant difference in scratching numbers between male (n=7) and female (n=2) monkeys for tested chemicals.

Mouse model of cholestasis was induced by ANIT (dissolved in corn oil) administration via oral gavage for 5 consecutive days at 25 mg/kg. Control mice received the same volume of corn oil. Animals were habituated to the testing environment for 2 d before baseline testing. The scratching behavior was recorded by a Panasonic video camera for 1 h every day before daily ANIT treatment.

To test whether LPC induces pain behavior at the spinal cord level directly, LPC was i.t. injected into the subarachnoid space with a total volume of 5 μL at a constant rate of 10 μL/min. Mechanical pain behavior was assessed with electronic von Frey filaments (Ugo Basile, Italy). Animals were habituated to the testing environment daily for at least 2 days before baseline testing. Mice were placed on a 5×5-mm wire-mesh grid floor in individual compartments and allowed to adapt for 0.5 h prior to the von Frey test. The von Frey filament was then applied to the middle of the plantar surface of the hind paw and the withdrawal responses following the stimulation were measured 3 times and averaged. Data on mechanical threshold was express as % of change.

Cell culture and transfection. HEK293 cells (ATCC® CRL-1573) were cultured on poly-D-lysine coated coverslips in 24-well plate containing DMEM media with high-Glucose (Gibco, Gaithersburg, MD) supplemented with 10% fetal bovine serum (FBS, HyClone Laboratories, Logan, UT) and 100 U/mL of penicillin/streptomycin (Gibco, Gaithersburg, MD). Cell cultures were maintained with 5% of CO₂ in a humidified incubator at 37° C. HEK cells were transfected with rat or human wild type or mutant TRPV4 channels with EGFP or YFP coupled to their C-termini (1.5 μg of plasmid for excised patch clamp and Ca²⁺ imaging and 200 ng for single-channel experiments). The jetPEI™ Polyplus transfection reagent (Polyplus Transfection, New York, NY) for electrophysiology or Lipofectamine™ 2000 Reagent (Invitrogen, Waltham, MA) for Ca²⁺ imaging were used for transfections per manufacturer's instructions as previously described (Chen et al. Pain 2014, 155, 2662-2672; Morales-Lazaro et al. Nat. Commun. 2016, 7, 13092). To investigate whether miR-146a can directly activate TRPV1 channels or indirectly via TLRs, HEK293 cells were transfected with rTRPV1 or co-transfected rTRPV1 with rTLR7, rTLR2, or rTLR6 (Addgene, Watertown, MA) for Ca²⁺ imaging assay. Control cells were transfected with GFP or YFP.

Primary mouse keratinocytes were cultured following previous protocol (Chen et al. J. Biol. Chem. 2016, 291, 10252-10262). The epidermis from the back skin of newborn WT or keratinocyte-Trpv4 cKO mice was separated from the dermis by floating the skin on 0.25% trypsin (Gibco, Gaithersburg, MD) for 14-18 h at 4° C. Basal keratinocytes were separated from the cornified sheets by filtration through a 70 μM cell strainer. Keratinocytes were plated on collagen I-coated dishes or glass coverslips and grown in EME media (Gibco, Gaithersburg, MD) containing 8% chelex (Bio-rad, Hercules, CA)-treated FBS with the final Ca²⁺ adjusted to 0.05 mM, bovine pituitary extract (50 μg/mL), epidermal growth factor (5 ng/mL), and lx antibiotics/antimycotics (Gibco, Gaithersburg, MD) in a humidified incubator with 5% CO₂ at 37° C. for 5-7 days until use. To knockdown Trpv4 in isolated keratinocytes from newborn keratinocyte-Trpv4 cKO mice, cells were treated with 4-OH tamoxifen at 500 nM for 72 h.

Primary human keratinocytes were cultured as previously described (Chen et al. J. Biol. Chem. 2016, 291, 10252-10262). In brief, surgically discarded foreskin samples, obtained from Duke Children's Hospital in accordance to institutionally approved IRB protocol, were incubated with Dispase (Gibco, Gaithersburg, MD, 4 U/mL) for 12-16 h at 4° C. followed by 0.05% trypsin (Gibco, Gaithersburg, MD) for 10-20 min at 37° C. Cells were maintained in keratinocyte serum-free media (Invitrogen, Waltham, MA) with 5% CO₂ at 37° C. and used at passage 2-3.

Primary mouse sensory neurons were cultured following previous protocol (Chen et al. Pain 2014, 155, 2662-2672). DRGs from 2-3 weeks old male WT, Trpv1 KO, and Trpa1 KO mice were dissected and digested with 1 mg/mL collagenase (Worthington, CSL1) and 5 mg/mL dispase (Invitrogen, Waltham, MA) for 1 h, then triturated. The resulting cell suspension was filtered through a 70 μm cell strainer (BD Biosciences, Bedford, MA) to remove debris. Neurons were cultured in DH10 medium (1:1 DMEM:Hams-F12, Invitrogen, Waltham, MA) with 10% FBS (Sigma, St. Louis, MO), 100 U/mL penicillin and 100 μg/mL streptomycin (Gibco, Gaithersburg, MD) and 50 ng/mL nerve growth factor (NGF; USBiological) on coverslips coated with poly-D-lysine and laminin (Invitrogen, Waltham, MA), and incubated with 5% CO₂ at 37° C. Ca²⁺ imaging was performed next day after culture.

Modeling of LPC binding to TRPV4 channels. The TRPV4 structure from Xenopus tropicalis (PDB file 6BBj; Deng et al. Nat. Struct. Mol. Biol. 2018, 25, 252-260) was used within the Schrodinger software package. Within Schrodinger, the protein preparation wizard within the Maestro Molecular Modeling Interface was run, and a conformer library of LPC 18:1, was generated using the ligand preparation tool. Within the Schrodinger software tool, the TRPV4 protein structure was minimized using the optimized potentials for liquid simulations model force field with standard parameters (OPLS, version 3e) (Harder et al. J. Chem. Theory Comput. 2016, 12, 281-296). The receptor grid generation was centered on residue K750 using the maximum 36 Angstrom distance. Ligand-receptor docking was performed using the Glide software platform (Friesner et al. J. Med. Chem. 2004, 47, 1739-1749).

Site-directed mutagenesis of TRPV4 channels. TRPV4 sequences of multiple species were aligned using Clustal Omega program. Site-directed mutagenesis to generate point mutations in rat and human TRPV4 channels were carried out using Phusion DNA polymerase enzyme (Salazar et al. Nat. Struct. Mol. Biol. 2009, 16, 704-710; Rosenbaum et al. Neuron 2002, 33, 703-713; Hsieh et al. Methods Mol. Biol. 2013, 978, 173-186), with final sequencing to check for presence of the induced mutation. We introduced the following mutations into the rTRPV4 or hTRPV4 channel: R746C, R746G, R746D, K754G, R757G, R774G, and W776G.

In vitro and ex vivo Ca²⁺ imaging. Routine procedures were followed for Ca²⁺ imaging in cultured DRG sensory neurons, epidermal keratinocytes, and HEK cells (Chen et al. J. Biol. Chem. 2016, 291, 10252-10262; Chen et al. Pain 2014, 155, 2662-2672). Ca²⁺ imaging of cultured cells in response to chemicals was conducted after loading with 5 μM Fura2-AM (Invitrogen, Waltham, MA) for 45 min after a ratiometric Ca²⁺-imaging protocol with 340/380-nm blue light for dual excitation. Ratios of emissions were acquired at 0.5 Hz. ΔR/R₀ was determined as the fraction of the increase of a given ratio over baseline ratio divided by baseline ratio. To investigate the effects of the TRPV4 inhibitors GSK205 or HC067047, the PLC inhibitor U73122, the Gag G Protein inhibitor BIM46187, the Gβγ G protein inhibitor Gallein, the TRPV1 inhibitor SB366791, the TRPA1 inhibitor HC030031, and IPP for TRPV3, on LPA-induced Ca²⁺ influx, LPC-induced Ca²⁺ influx, or miR-146α-induced Ca²⁺ influx, cells were incubated with the inhibitors for 15 min before stimulation. Control cells received vehicles.

A previously established method was followed for ex vivo Ca²⁺ imaging of DRG explants (Kim et al. Neuron 2014, 81, 873-887). Intact DRGs (L4 or L5) were isolated from naïve male or female Pirt-GCaMP3 mice (2-3 months old) and equilibrated in artificial cerebrospinal fluid (ACSF) bubbled with 95% O₂/5% CO₂ at room temperature. After 15 min, explants were placed in a dish with 2 mL of pre-oxygenated ACSF and imaged using a Zeiss 780 upright confocal microscope (Carl Zeiss AG, Oberkochen, Germany) with 20× water immersion objective and Z-stack approach at the 488 nm wavelength. Explants were stimulated by miR-146a or miR-146a scramble. After 15 min recording, capsaicin was then applied to identify whether the miR-146a responding neurons were TRPV1 positive. In addition, to examine whether inhibition of TRPV1 or TRPA1 ion channels attenuates miR-146a-induced Ca²⁺ signal, explants were pretreated with the TRPV1 inhibitor SB366791 or the TRPA1 inhibitor HC030031 during the 15 min sample equilibration. Ca²⁺ fluorescence intensity was determined using the ImageJ software (NIH, Bethesda, MD, USA). For each neuron, the pixel intensity (Fₜ) was assessed for each frame and the pixel intensity recorded from the first 20 frames was taken to determine the average baseline value (F₀). Ca²⁺ signal amplitudes are presented as ΔF/F₀, which is the ratio of fluorescence difference (Fₜ−F₀) to baseline (F₀).

Electrophysiology. Currents were recorded using the inside-out configuration of the patch-clamp technique (Hamill et al. Pflugers Arch. 1981, 391, 85-100). Solutions were changed with a RSC-200 rapid solution changer (Molecular Kinetics). GSK1016790A was prepared in DMSO at 15.25 mM for the stock, which was kept at −20° C. and diluted to 1 μM in recording solution for application to membrane patches. LPC 18:1 was prepared in DMEM-BSA 0.1% at 10 mM for stock solutions, kept at −70° C. and diluted in recording solution. The recording solutions contained (in mM): 130 NaCl, 3 HEPES (pH 7.2) and 1 EDTA for the bath and 130 NaCl, 3 HEPES (pH 7.2) and 5 CaCl₂ in the pipette. Experiments were performed at room temperature. Mean current values in response to GSK1016790A or LPC 18:1 were measured after channel activation had reached the steady-state (˜3 min). Currents were obtained using voltage protocols where the holding potential was 0 mV and 10 mV steps from −120 to 120 mV or from −60 to +60 mV for 100 ms, to 0 mV. Borosilicate glass was used for pipette fabrication (5 Me)). Currents were low-pass filtered at 2 kHz and sampled at 10 kHz with an EPC 10 amplifier (HEKA Elektronik) and were plotted and analyzed with Igor Pro (Wavemetrics Inc.).

For single-channel recordings, Borosilicate glass 30 MS) pipettes were used. Recordings were obtained at +60 mV by acquiring several traces of 1-3 s duration. The effect of GSK1016790A or LPC 18:1 on single TRPV4 channels was studied in inside-out. Currents were filtered at 2 kHz and sampled at 5 kHz. Patches containing only one channel activated by different compounds were identified as those that did not contain overlapping opening events. Single-channel openings and closures were identified with the half-threshold crossing technique (Morales-Lazaro et al. Nat. Commun. 2016, 7, 13092). The channel open probability was calculated as the sum of the total open time divided by the sweep duration. Dwell times and amplitude histograms in the closed or open states were collected in logarithmic time histograms according to the Sine-Sigworth transformation (Sigworth et al. Biophys. J. 1987, 52, 1047-1054). Sums of three or two exponential components were fitted to histograms using a least-squares algorithm.

In vitro interaction assays of LPC-TRPV4. Surface proteins were obtained from HEK293 cells transiently expressing rTRPV4-EGFP channels using the Pierce Cell Surface Isolation kit (Pierce Biotechnology, Rockford, IL) following the manufacturer's instructions. Overlay assays were performed as previously described (Morales-Lazaro et al. Nat. Commun. 2016, 7, 13092). In brief, LPC 18:1 was spotted (200 pmol per spot) onto a nitrocellulose membrane (GE Healthcare, Pittsburgh, PA) and then blocked with 1% fatty acid-free BSA (Calbiochem) and 6% fat-free dried milk in PBS. Membranes were then incubated with the surface protein solutions and exposed to anti-GFP antibody (Sigma, St. Louis, MO) diluted 1:1000 in 3% fat-free dried milk in PBST (with 0.1% of Tween). Membranes were incubated with horseradish peroxidase-conjugated secondary anti-rabbit antibody (Cell Signaling Technology) diluted 1:7500 in 6% fat-free dried milk in PBST. The binding of rTRPV4-EGFP and TRPV4-R746D-EGFP to the lipid-containing spots was visualized by chemiluminescence by exposing the blot for 15 min (Amersham Bioscience, Piscataway, NJ). Semi-quantitative densitometric analysis was done using ImageJ (NIH) and expressed as relative protein levels of TRPV4 bound to each spot.

Western Blot. Routine procedures were followed (Chen et al. J. Biol. Chem. 2016, 291, 10252-10262, Chen et al. Pain 2014, 155, 2662-2672). Briefly, cultured keratinocytes and dissected dorsal neck skin (0.5×0.5 cm, the area that received the treatment) were protein-extracted in radioimmunoprecipitation assay (RIPA, Sigma, St. Louis, MO) buffer and electroblotted to polyvinylidene fluoride (PVDF) membranes after gel separation of proteins in a 4-15% polyacrylamide gel (Bio-Rad, Hercules, CA). Membranes were blocked with 5% BSA in TBST and incubated with primary antibodies rabbit anti-pERK or anti-ERK (both at 1:2000) followed by secondary antibody (anti-rabbit peroxidase-conjugated, 1:5000; Jackson ImmunoResearch), and chemiluminescence substrate (ECL-Advance, GE Healthcare). Immunoblot band intensity was quantitated using the software Image J and ERK served as a control for pERK expression.

Immunohistochemistry and morphometry analysis. Routine procedures were followed (Chen et al. Pain 2014, 155, 2662-2672). Briefly, mice were perfused transcardially with 0.01 M PBS followed by ice-cold 4% paraformaldehyde (PFA, Sigma, St. Louis, MO). Cervical spinal cord, TGs and cervical DRGs, and dorsal neck skin were dissected and post-fixed in 4% PFA overnight, cryoprotected in 20% sucrose (48 h) and sectioned on a cryostat (30 μm for spinal cord, 12 μm for TG and DRG and neck skin). Sections were blocked with 5% normal goat serum (Jackson), and incubated overnight with primary antibodies: rabbit anti-TRPV1 (1:5000), TRPA1 (1:200), TRPV4 (1:300) or p-MEK (1:200). Immunodetection was accomplished with secondary antibodies (AlexaFluor 594-conjugated goat anti-rabbit) for 2 h, and cover-slipped with Vectashield (Vector). DAPI (1 μg/mL) was used for counterstaining with p-MEK in skin sections. Digital micrographs were acquired using a BX61 Olympus upright microscope with a high-resolution ORCA-Spark camera (Hamamatsu) and with constant acquisition/exposure settings, using CellSens Dimension software (Olympus). 4-6 sections were analyzed per mouse. TG and DRG neurons were identified by morphology. The cutoff density threshold was determined by averaging the density of three neurons per section that were judged to be minimally positive, using ImageJ software. All neurons for which the mean density exceeded the threshold≥25% were judged as positive. Positive cells were expressed as % of total counted TG neurons. The labeling density of TRPV1 in spinal cord was measured using the integrated density algorithm of Image J.

Measurement of released vesicles and extracellular miR-146a from cultured keratinocytes or sera Medium for the cultured keratinocytes was replaced with serum-free medium 2 h before LPC stimulation. Fifteen minutes after LPC, the supernatant of the cells was harvested and subjected to two steps of centrifugation (i) 300 g for 5 min to eliminate remaining cells, (ii) 16,000 g for 30 min to eliminate cell debris and apoptotic bodies. Finally, cell-free supernatants were further purified using a Vesicular Isolation kit (Invitrogen, Waltham, MA) according to the manufacturer's instructions, with final product resuspended in ice-cold PBS. Total RNA extraction was then carried out using a Total RNA Isolation kit (Invitrogen, Waltham, MA). Enrichment for small RNAs was carried out by sequential increase in ethanol concentration and passing through glass-fiber filters. RNase free water was used to elute small RNAs in the final elution step. For human PBC sera or sera from ANIT-treated mice, RNA was isolated using Qiagen miRNeasy Plasma/Serum kit. cDNA synthesis from extracted RNAs was performed according to manufacturer's instructions (qSTAR miRNA kit, Origene). For qPCR, the stem-loop oligonucleotides specific for the following miRs are as follows: miR-146a-5p (GAGAACTGAATTCCATGGG, SEQ ID NO: 4), miR-let-7b (GAGGTAGTAGGTTGTGTGG, SEQ ID NO: 5), miR-125b-1 (CCCT GAGACCCTAACTTG, SEQ ID NO: 6), miR-203 (GTGGTTC TTGACAGTTCAAC, SEQ ID NO: 7), and miR-16-5p (AGCAGCAC GTAAATATTGGC, SEQ ID NO: 8). Primers were purchased from Integrated DNA Technologies company (Coralville, IA). qPCR reactions for each sample were run in triplicates, including no-template controls. MiR-16-5p was selected as a control due to the relative constancy of its expression in various cultured cell lines (Schwarzenbach et al. Clin. Chem. 2015, 61, 1333-1342). qPCR for this miR was performed in tandem with target miRs to determine the optimal normalization procedure. To investigate the effects of the specific TRPV4 inhibitors GSK205 or HC067047 and the specific MEK inhibitor U0126 on LPC-induced extracellular release of miR-146a, cells were incubated with the inhibitors for 15 min before stimulation. Control cells received the same volume of vehicle.

Vesicular release from cultured keratinocytes was quantified by detecting acetylcholinesterase (AChE) activity in the extracellular release fluid. (Gupta et al. Am. J. Physiol. Heart Circ. Physiol. 2007, 292, H3052-3056; Malik et al. Methods Mol. Biol. 2016, 1448, 237-248). CBQCA Protein Quantitation Kit (Molecular Probes, Eugene, OR) was used to assess the total protein amounts of each sample. Quantitation was carried out according to kit instructions (Fluorocet, Systems Biosciences, Palo Alto, CA). Briefly, vesicles were lysed to release esterase enzyme whose activity is measured using a florescence dye, excitation at 544 nm and emission at 590 nm. Fluostar Optima (BMG Labtech, Cary, NC) microplate reader was used to measure esterase activity. 500 ng protein equivalent of input was used per well. To determine the contribution of Rab5a and Rab27a to LPC-induced miR-146a release, cells were pre-treated with the Rab5a (5′-GUAGAAUCAA GUUUCUAAUUCUGAA-3′ SEQ ID NO: 9, 5′-UUCAGAAUUAGAAACUUGAUUCUACCA-3′ SEQ ID NO: 10) or Rab27a (5′-AGCUAAAA CUGAGAGCUUCAAACAG-3′ SEQ ID NO: 11, 5′-CUGUUUGAAGCUCUCAGUUUUAGCUUA-3′ SEQ ID NO: 12) siRNA (IDT, Coralville, IA) for 72 h before stimulation. Control cells were treated with scramble siRNA.

LPC measurement in sera and skin. Blood and dorsal neck skin (˜0.5×0.5 cm) were harvested from ANIT- or control-treated mice at day 5. Blood was drawn via cardiac puncture and allowed to clot at room temperature for 20 min. After centrifugation at 2000×g for 10 min at 4° C., serum was collected and stored at −80° C. until use. After weighing, skin was cut into pieces and sonicated in methanol for 1 min at 4° C. After sonication, samples were centrifuged at 12000×g for 15 min at 4° C. and supernatant was collected and stored at −80° C. until analysis. Total levels of LPC in mouse serum and skin were determined by an enzymatic colorimetric method (Kishimoto et al. Clinical biochemistry 2002, 35, 411-416). In brief, 8 μL of samples were treated with lysophospholipase, glycerophosphorylcholine, phosphodiesterase, and choline oxidase. The resulting hydrogen peroxide generated was quantified using horseradish peroxidase and TOOS reagent. The absorbance was measured by microplate reader (Molecular Devices, San Jose, CA). Total level of LPC was detected at around 1 mM in sera of control mice.

Serum levels of LPC in PBC patients were determined by the AbsoluteIDQ™ p180 kit (Biocrates, Life Sciences AG, Innsbruck, Austria) following the manufacturer's instructions. The assay allows simultaneous quantification of 188 metabolites out of 10 μL serum, including 14 species of LPC: 14:0, 16:0, 16:1, 17:0, 18:0, 18:1, 18:2, 20:3, 20:4, 24:0, 26:0, 26:1, 28:0, and 28:1. In brief, after 10 μL internal standards or 10 μL serum were added into the filter wells of a 96-well plate, the wells were dried by nitrogen, and 50 μL of 5% phenyl-isothiocyanate solution was added into each well for derivatization. After incubation, the wells were dried again, and 300 μL of methanol containing 5 mM ammonium acetate were added into each well to extract the metabolites. The extract was then centrifuged into the collection wells, and each well was diluted with 300 μL of the running solvent (a proprietary mixture provided by Biocrates, Innsbruck, Austria). The flow injection analysis-tandem mass spectrometry (FIA-MS/MS) technique was used to detect 14 species of LPC. Using electrospray ionization in positive mode, samples were introduced directly into a Xevo TQ-S triple quadrupole mass spectrometer (Waters) operating in the Multiple Reaction Monitoring (MRM) mode. MRM transitions (compound-specific precursor to product ion transitions) for each analyte and internal standard were collected over the appropriate retention time. The FIA-MS/MS data were analyzed using Biocrates MetIDQ™ software. Internal standards and quality control samples of the p180 Kit were utilized to benchmark the quality of the assay and the robustness of the data. For all analyzed LPC species, data were expressed in relative concentrations. Of note, LPC 14:0 data was not shown since it was below the lower limit of detection. Total level of 14 LPC species as above was detected at around 155 μM in sera of PBC patients without itch.

Quantitative real-time PCR. Total RNA from TGs or cervical DRGs was prepared using Directzol RNA kit (Zymo Research) following the manufacturer's instructions. RNA was aliquoted and stored at −80° C. until use. 1 μg of total RNA was reverse transcribed using SuperScript III Reverse Transcriptase (Invitrogen, Waltham, MA). Real-time PCR was performed with equal amounts of cDNA in the GeneAmp 7700 sequence detection system (Applied Biosystems) using QuantiTect SYBR Green PCR Kit (Qiagen). The OOCt method was used for relative quantification of gene expression. Primers were synthesized by Integrated DNA Technologies (Coralville, IA) and their sequences were: internal control 11-tubulin (forward: 5′-CCTG CCTTTTCGTCTCTAGC CGC-3′ SEQ ID NO: 13, reverse: 5′-GCTGATGACCTCCCA GAACTTGGC-3′ SEQ ID NO: 14) and TRPV4 (forward: 5′-GTGGGCAAGAGCTCAGATGGCACTC-3′ SEQ ID NO: 15, reverse: 5′-CCACCGAGG ACCAACGATCCCTAC G-3′ SEQ ID NO: 16).

Statistical analysis. All data are expressed as mean±SEM. Two tailed t-test and one-way or two-way ANOVA followed by Tukey's post-hoc test were used for group comparison (SPSS, version 25). For scratching behavior in nonhuman primates, analyses of repeated measures data were performed using a linear mixed model as implemented in SAS 9.4. The correlated nature of repeated measures was taken into account by using an autoregressive correlation matrix in the model specification. Between-group contrasts were evaluated for statistical significance using an F-statistic. Pearson's correlation coefficient and corresponding p value were calculated to assess the correlation between itch intensity and LPC and miR146a levels. p<0.05 indicated statistically significant differences.

### Example 2

### LPC Induces Itch Via Keratinocyte TRPV4

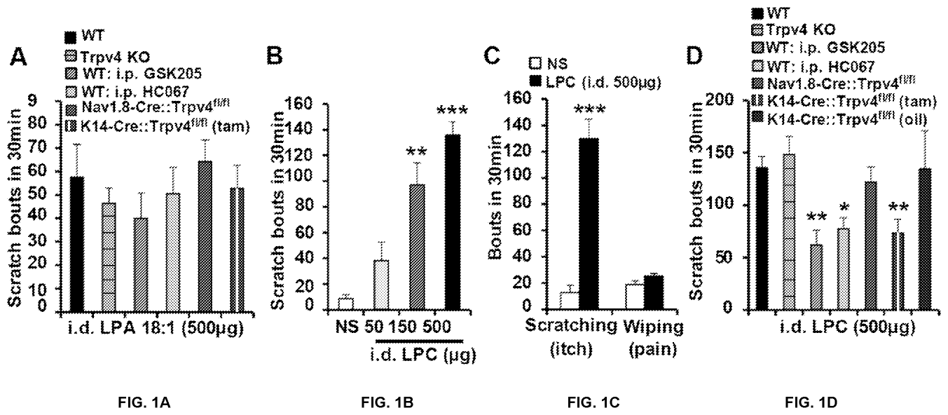

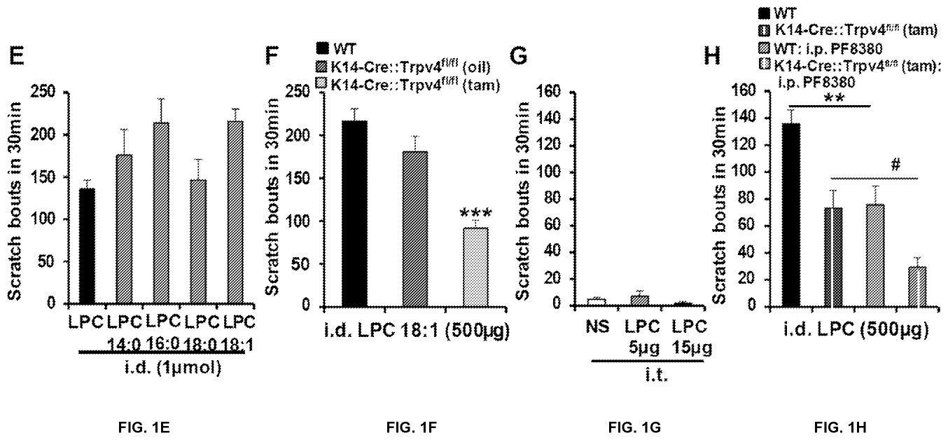

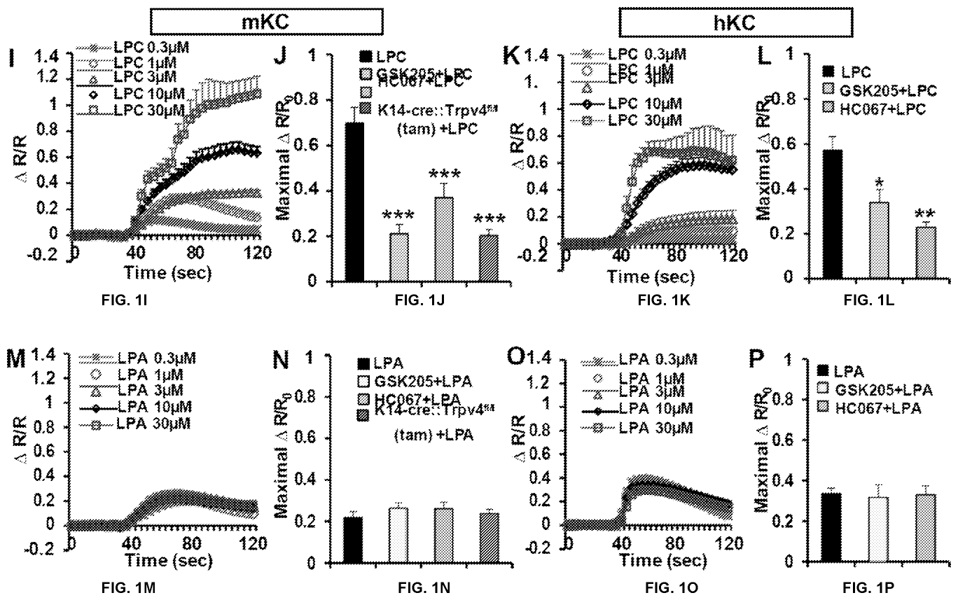

First, we addressed whether the known cholestatic pruritogen, LPA, induces pruritus via TRPV4. In wild type (WT) mice, intradermal (i.d.) injection of 500 μg LPA (18:1) (dose following previous studies (Kremer et al. Gastroenterology 2010, 139, 1008-1018; Kittaka et al. J. Physiol. 2017, 595, 2681-2698) into the dorsal neck elicited moderate scratching. We recorded no significant change in scratching when using Trpv4 pan-null, skin keratinocyte-conditional, and sensory neuron-conditional Trpv4^{fl/fl} (FIGS. 9A-9D) KO lines. We also intraperitoneally (i.p.) administered two different TRPV4-selective inhibitors GSK205 and HC067047 and recorded no significant reduction in scratching in response to LPA (FIG. 1A). Thus, we found no evidence that LPA's pruritogenicity relies on TRPV4.

Next we addressed whether LPC, LPA's direct metabolic precursor, can evoke scratching behavior. LPC was found elevated in blood and lesional skin in some pruritic diseases, raising the question whether it elicits itch via an effect on skin. In humans, LPC was observed as an irritant upon i.d. injection (itch was not assessed in that study) and was shown to lower nociceptive thresholds upon intrathecal (i.t.) injection. These pain-related observations were linked to LPC affecting TRPC5 and TRPM8. Therefore, the pruritogenic effects of LPC was addressed. We observed a dose-dependent pruritogenic response to i.d. injection of egg-LPC (LPC; a mixture of LPC species, see Example 1, FIG. 1B) in mice. We chose to use this dose-range and view it as pathophysiologically relevant because of high systemic LPC levels present in vivo. Of note, LPC at 500 μg elicited more than twice as many scratch bouts as did 500 μg LPA (135 vs 58 scratch bouts in 30 min). Using a mouse cheek model (Shimada et al. Pain 2008, 139, 681-687), we found that mice responded to 500 μg LPC i.d. with a pruritus-related scratching and not a pain-related wiping behavior (FIG. 1C).

We next addressed whether LPC-induced scratching was TRPV4-dependent. We observed no significant change in LPC-induced scratching in WT vs pan-null Trpv4 KO mice, but we saw a reduction when inhibiting TRPV4 systemically by i.p. injection of GSK205 and HC067047 (FIG. 1D). We reasoned that acute inhibition of TRPV4 indicates TRPV4-dependence of LPC-evoked scratching that was masked in pan-null Trpv4 KO mice likely because of gene-regulatory compensation. To resolve this issue, we investigated LPC-evoked scratching in Trpv4 conditional knockout (cKO) mice. Sensory neuron-Trpv4 cKO (Nav1.8-Cre::Trpv4^{fl/fl}) did not exhibit different scratching behavior induced by LPC, whereas tamoxifen-induced keratinocyte-Trpv4 cKO mice (K14-Cre-ER^{tam}::Trpv4^{fl/fl}) showed a >50% reduction (FIG. 1D). These findings indicate that skin keratinocyte-TRPV4, but not sensory neuron-TRPV4, is needed for LPC-evoked scratching. Different species of LPC at the same dose induced robust scratching responses (146-216 scratch bouts in 30 min), with LPC (18:1) the most potent (FIG. 1E). In addition, LPC (18:1)-induced itch was also reduced in keratinocyte-Trpv4 cKO mice (FIG. 1F).

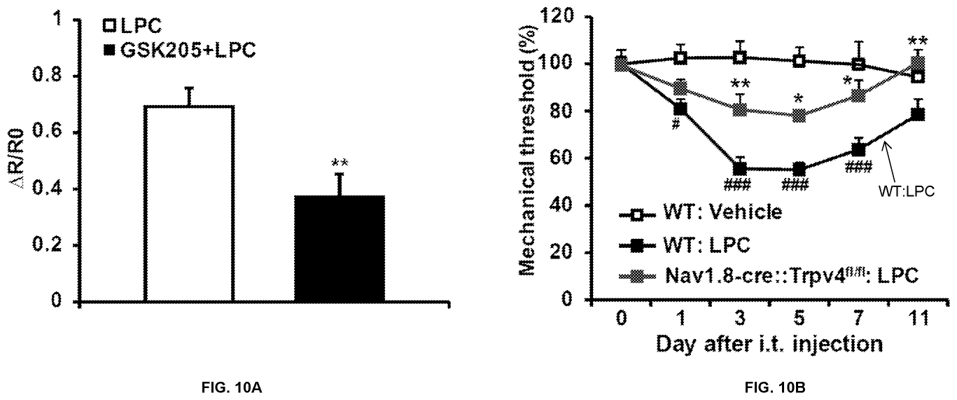

In view of the established pro-algesic roles of i.t. LPC, we addressed specifically whether i.t. LPC elicits scratching. It did not at doses of 5 μg and 15 μg (FIG. 1G), whereas it evoked long-lasting pain at days 1-7, after administration of 15 μg (FIG. 10A, FIG. 10B). Interestingly, sensory neuron-Trpv4 cKO mice did not have less scratching than WT in response to i.d. LPC (FIG. 1D) but showed attenuated pain caused by i.t. LPC. In addition, LPC caused increased Ca²⁺ transients in WT sensory neurons, which was significantly attenuated when inhibiting TRPV4 (FIG. 10A, FIG. 10B). These results suggest that LPC intradermally elicits itch via a non-neuronal peripheral mechanism critically involving keratinocyte-TRPV4, whereas LPC intrathecally relies on sensory neuron-TRPV4 to evoke pain.

Next, we wanted to determine whether the LPC→LPA conversion contributes to pruritogenicity of LPC. If so, we expected the attenuated LPC scratch response in keratinocyte-Trpv4 cKO mice to be suppressed further when inhibiting the LPC→LPA conversion because no LPA is made while LPC's molecular target in keratinocytes, TRPV4, was selectively knocked down. We did this by i.p. injection of the potent autotaxin inhibitor PF8380. Confirming our hypothesis, residual LPC-induced scratching in keratinocyte-Trpv4 cKO mice was reduced, further indicating that the LPC≥LPA conversion is relevant for scratching in vivo, and that LPC, but not its “co-pruritogen” metabolite LPA, signals directly via TRPV4 (FIG. 1H).

### Example 3

### LPC Triggers Ca²⁺ Influx Via TRPV4 Channels in Mouse and Human Keratinocytes

In vivo behavior suggested LPC-induced TRPV4 activation in skin keratinocytes, as detailed above. Therefore, we interrogated primary skin keratinocytes from both mouse and human for their TRPV4 activation mechanisms. We demonstrated that LPC induced a dose-dependent Ca²⁺ increase in cultured mouse and human keratinocytes. This signal was significantly attenuated upon pretreatment with TRPV4-selective inhibitors, and also in keratinocytes derived from keratinocyte-Trpv4 cKO mice (FIG. 1I-FIG. 1L). In contrast, LPA, at the maximum concentration 30 μM, induced much reduced Ca²⁺ response versus LPC. Of note, dose dependence of the Ca²⁺-response to LPA was not appreciable, and there was no effect of loss-of-function of TRPV4 in mouse or human (FIG. 1M-FIG. 1P). Thus, at the cellular level in both mouse and human keratinocytes, LPC, but not LPA, produces TRPV4-dependent Ca²⁺ influx.

Since TRPV3 is also abundantly expressed in skin keratinocytes and contributes to itch, its possible involvement in LPC-evoked itch was tested. When inhibiting TRPV3, we recorded no significant change in Ca²⁺ transients evoked by LPC in mouse and human keratinocytes (FIG. 11A, FIG. 11B) and no significant reduction in scratching behavior elicited by i.d. LPC (FIG. 11C). These data suggested that the pruritogenic effects of LPC may not rely on TRPV3.

### Example 4

### LPC Activates TRPV4 Directly Via a C-Terminal Binding Pocket

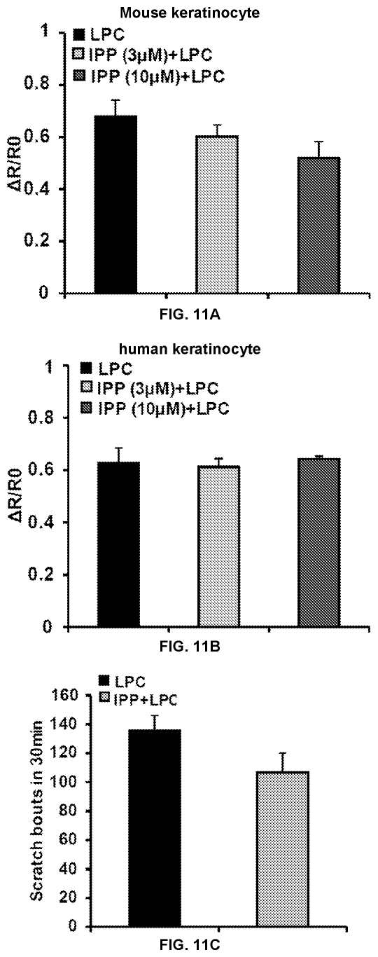

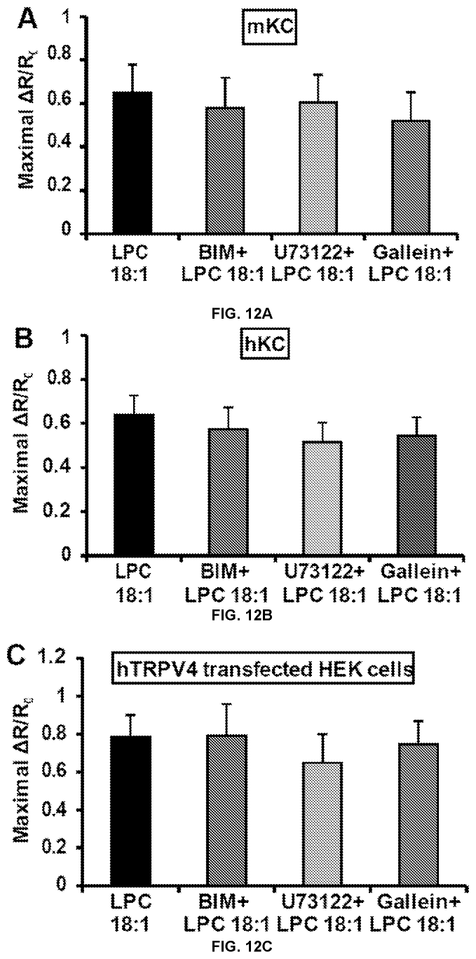

We next analyzed whether LPC activates TRPV4 by direct binding to the channel. LPC (18:1) was selected because it is: (1) one of the major bioactive LPC sub species; (2) the most potent species tested in scratching behavior (FIG. 1E); and (3) abundantly present in body fluids. We first tested inhibitory effects of the Gag inhibitor BIM-46187, the phospholipase-C inhibitor U73122, and the Gβγ inhibitor Gallein on LPC (18:1)-induced Ca²⁺ signals. We noticed no inhibitory effects of these compounds when applied to mouse and human keratinocytes and to human TRPV4 (hTRPV4)-expressing HEK293 cells (FIG. 12A-FIG. 12C). This suggests LPC does not act on a G protein-coupled receptor (GPCR) signaling system upstream of TRPV4.

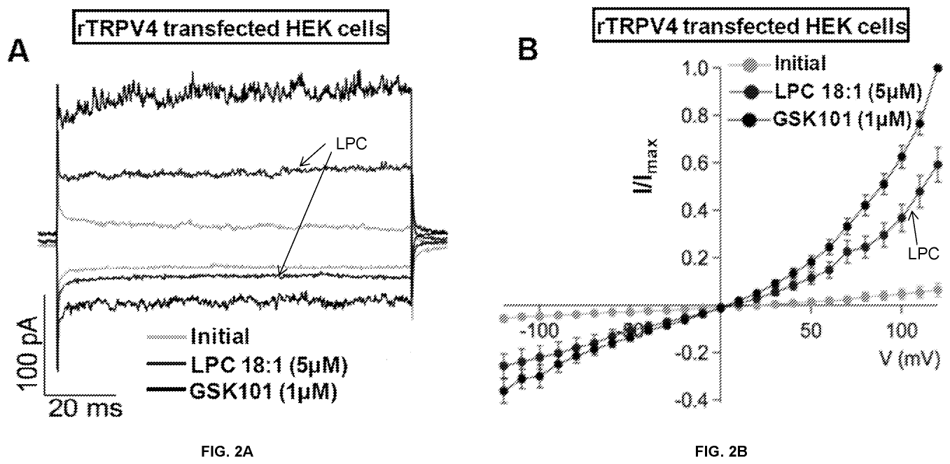

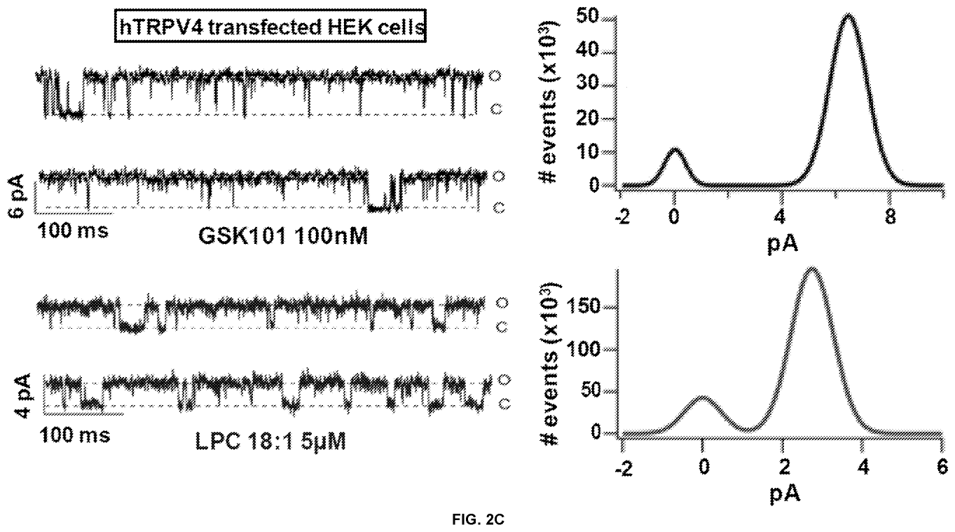

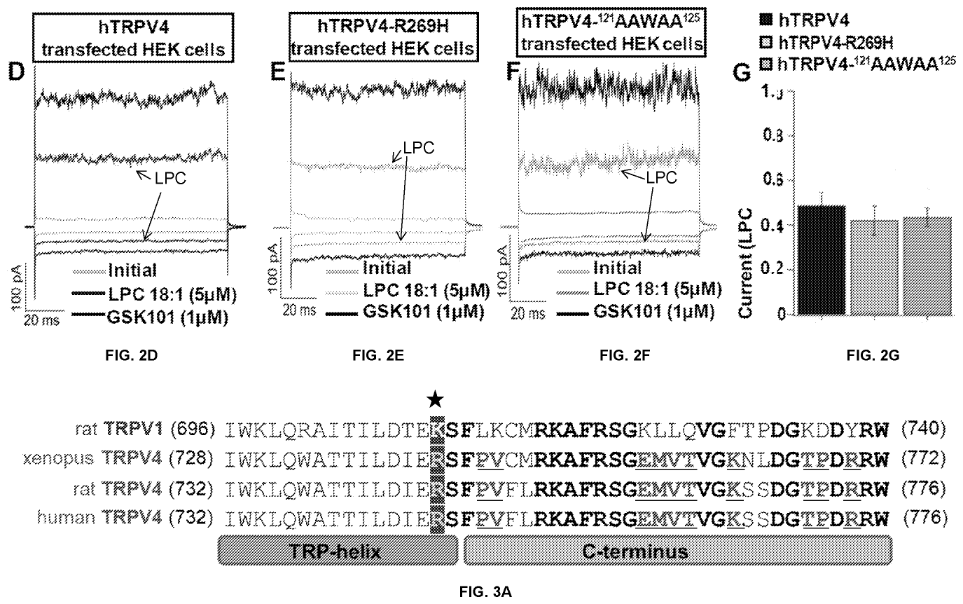

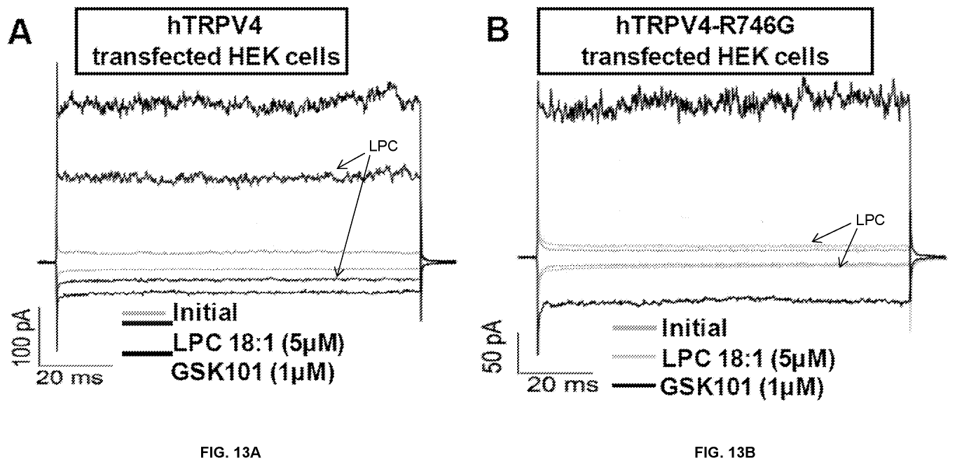

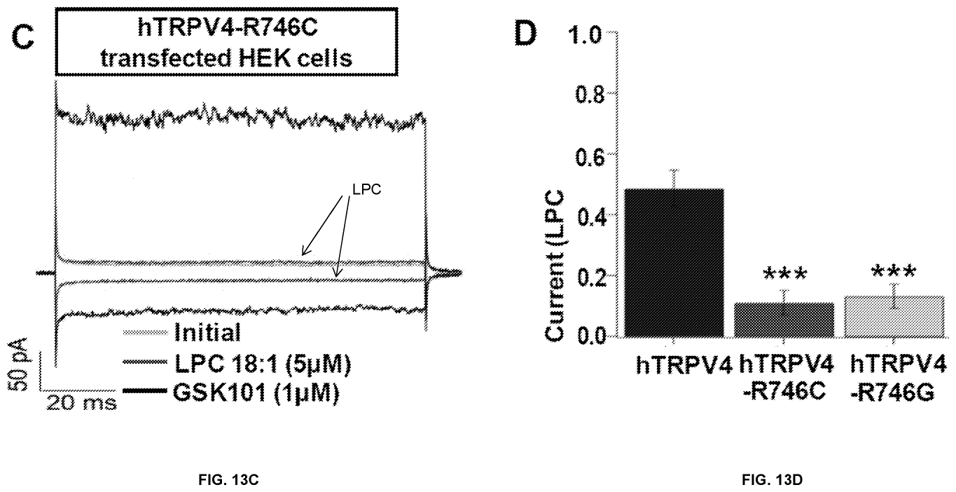

Based on this finding, we conducted patch-clamp recordings to evaluate TRPV4 channel activation. We used the inside-out configuration for recordings, in rat TRPV4 (rTRPV4)-transfected HEK293 cells using LPC (18:1, 5 μM) and the TRPV4 activator GSK101 (1 μM) as control. We demonstrated that LPC (18:1) applied to the excised patches produced activation at 54% of rTRPV4 currents when GSK101-induced currents were set at 100% (FIG. 2A-FIG. 2B). These currents were outwardly-rectifying both for LPC (18:1) and GSK101, recorded both for rat (FIG. 2A-FIG. 2B) and human (FIG. 13A, FIG. 13D) TRPV4. We also determined single channel conductance to further delimit the biophysical mechanism by which LPC (18:1) promotes an increase in hTRPV4-mediated currents. We first measured the open probability (Po) and the single-channel amplitude/conductance of hTRPV4 expressed in HEK293 cells in the presence of 100 nM GSK101 (FIG. 2C) and obtained a Po value of 0.85±0.08, an amplitude of 6.45±0.55 pA, and a single channel conductance (gi) of 107 pS at +60 mV. Notably, when we performed the same experiment using 5 μM LPC (18:1, FIG. 2C), we obtained a Po of 0.75±0.08, similar to that of GSK101, but an amplitude of 3.27±0.41 pA and a gi of 54.5 pS. These single channel data provide a biophysical explanation for our observation that activation of TRPV4 by LPC (18:1) accounted for ˜50% of the GSK101 current.

Based on previous observations that LPA interacts with the PIP₂-binding site of TRPV1, we also tested whether hTRPV4 with mutations in PIP₂-interaction sites affect activation by LPC (18:1). TRPV4 channels with mutations R269H and 121AAWAA125 responded in the same manner as hTRPV4 (WT) channels, demonstrating that these sites are not required for activation by LPC (18:1, FIG. 2D-FIG. 2G).

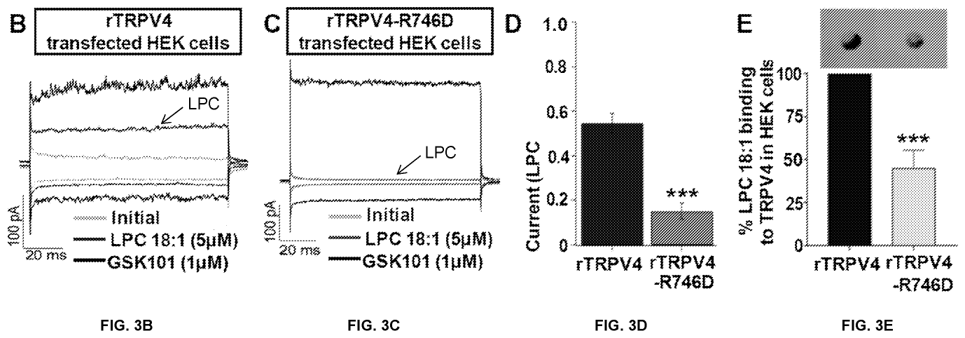

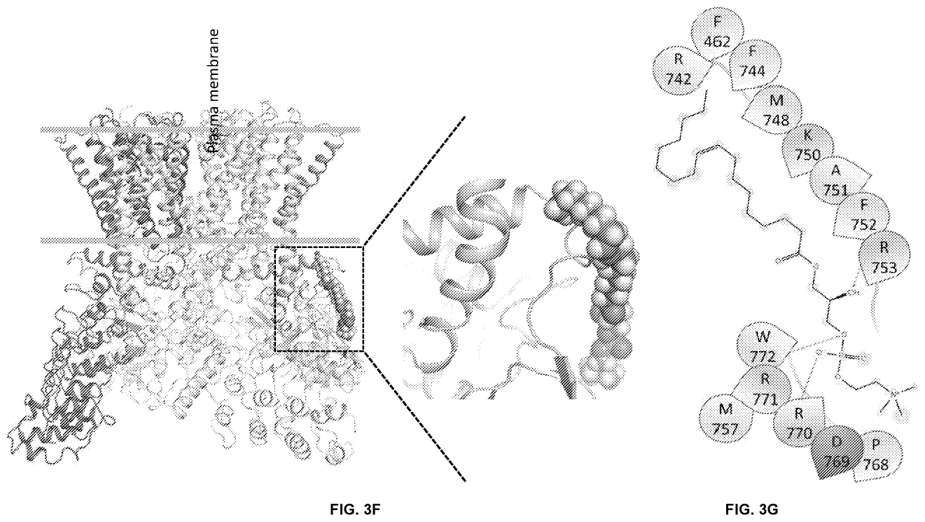

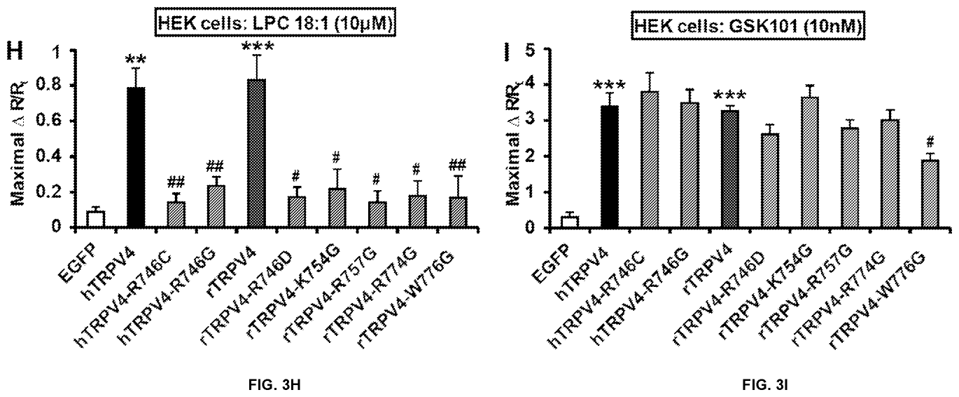

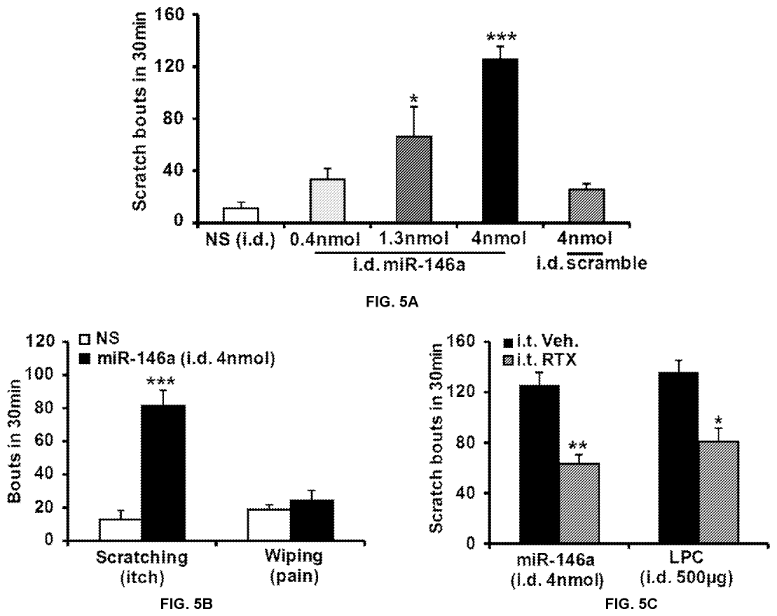

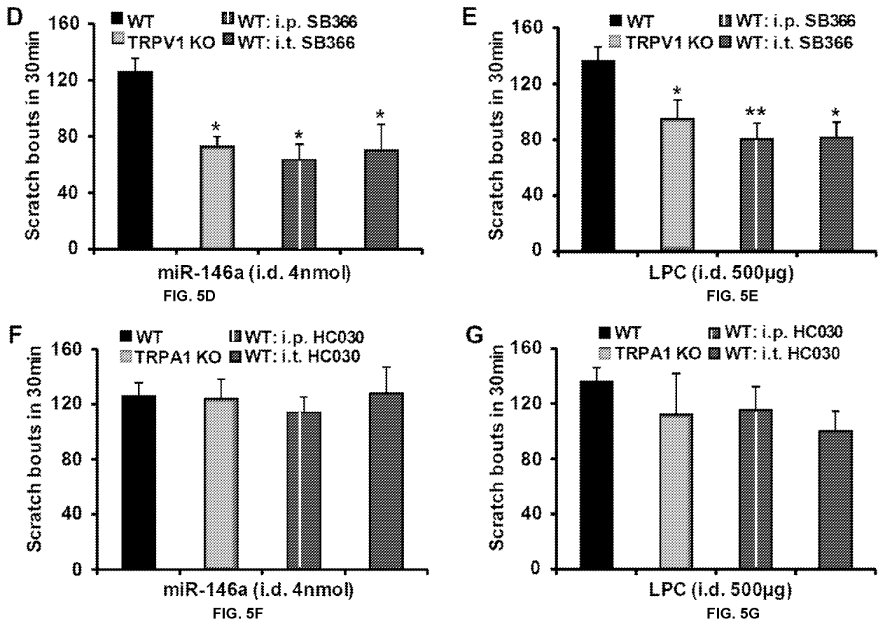

TRPV1 activation by LPA relies on the C-terminal K710 residue. We therefore aligned TRPV4 from rat, human, and xenopus (the latter used for the TRPV4 structure; Deng et al. Nat. Struct. Mol. Biol. 2018, 25, 252-260) with the TRP-helix and C-terminal domain of rTRPV1 (FIG. 3A). We identified r/hTRPV4 (R746) and xenTRPV4 (R742) as aligning with rTRPV1 (K710). Of note, these positively charged residues were located C-terminally within the highly conserved TRP-helix. We then investigated whether R746 (mammalian TRPV4) is important for the channel to be activated by LPC (18:1). We generated affirmative electrophysiological evidence by: (1) charge reversal mutation of arginine to aspartic acid: R746D (FIG. 5B-FIG. 5D); (2) mutagenesis of arginine to inert glycine: R746G (FIG. 13B, FIG. 13D); and (3) use of a human arginine to cysteine polymorphism: R746C (www.ncbi.nlm.nih.gov/clinvar/variation/VCV000450199.2, FIG. 13C, FIG. 13D). Using biochemical assays, we also demonstrated binding of LPC (18:1) to rTRPV4 and observed a significant reduction of this interaction with mutation R746D (FIG. 3B-FIG. 3E). Taken together, these results strongly support that mammalian R746, located C-terminally in the TRP-helix, is required for TRPV4 activation by LPC.

We used this information to build a structure-based model of LPC (18:1)/xenTRPV4 binding (FIG. 3F-FIG. 3G). In this model, the ε-N of K750 (K754 of mammalian TRPV4) was predicted to be in contact with the carbonyl oxygen of LPC (18:1). Multiple poses of LPC (18:1) were generated, each with this contact pair. However, the aliphatic chain of LPC (18:1) displayed mobility among the poses. Best fit was modeled using a computational docking simulation (see Example 1), and we ultimately predicted docking of LPC (18:1) to a TRPV4 C-terminal binding site as shown in FIG. 3F-FIG. 3G. Our structural simulation suggested a model in which LPC (18:1) docks to a groove of the channel from K750-W772 (mammalian K754-W776) (FIG. 3F-FIG. 3G).

Considering this structural prediction, we conducted mutagenesis by targeting positively charged residues K754, R757, R774, and W776 of rTRPV4. When replacing each residue with glycine, LPC (18:1) had significantly reduced potency to activate these mutagenized TRPV4 channels expressed in HEK293 cells (FIG. 3H), whereas GSK101 remained largely active (FIG. 31). This experiment supported LPC (18:1) docking as predicted by our structural model. Structural resolution of LPC isoforms bound to TRPV4 may provide further confirmation. In addition, the mutagenized sites did not affect GSK101 activating TRPV4, likely via a different binding site than LPC (18:1) (FIG. 14). Thus, we proposed a C-terminal binding site of TRPV4 for direct activation by LPC (18:1) as a potential molecular mechanism that initiates the interaction between the glycerophospholipid and the channel.

### Example 5

### TRPV4 Activation by LPC Induces ERK Phosphorylation, then Triggers Extracellular Release of miR-146a Through Rab5/Rab27a in Skin Keratinocytes

We next focused on intracellular signaling downstream of TRPV4 activation by LPC to determine the mechanism(s) by which activated keratinocytes relay the signal to skin-innervating peripheral sensory axons of pruriceptor neurons. We focused on MAP kinase signaling based on our previous observations that MEK-ERK activation can function downstream of TRPV4-mediated Ca²⁺-influx in keratinocytes in response to pruritogens and in human airway epithelia in response to air pollution. We detected a rapidly increased phosphorylation of ERK (pERK) in mouse and human primary keratinocytes 10 min after stimulation with LPC (FIG. 4A-FIG. 4B). This increase was TRPV4 dependent, as two TRPV4 inhibitors (GSK205, HC067047) eliminated the increase of pERK. In mouse dissected skin, we also observed an increase of pERK in response to i.d. LPC (30 min post-injection). Similar to results from primary keratinocyte cultures, this increase was eliminated in mice systemically treated with GSK205 and in keratinocyte-Trpv4 cKO mice (FIG. 4C). This indicated that TRPV4 is required for the increase of pERK in keratinocytes in response to LPC, both in isolated primary cells and live animals.

We hypothesized that inhibition of pERK in the skin would attenuate the behavioral response. Indeed, pretreatment with MEK-inhibitor U0126 (i.d.) caused significant reduction in LPC-induced scratching (FIG. 4D). Thus, formation of pERK in the skin would be necessary for LPC-evoked itch. Next, we addressed whether activation of MAP kinases in skin keratinocytes suffices to elicit formation of pERK and behavioral correlates of itch. To analyze this, we used mice with a constitutively active B-raf transgene, directed to express in keratinocytes by an inducible keratin-5 promoter. Upon transgene induction in skin keratinocytes with tamoxifen, the levels of pMEK (FIG. 4E) and pERK (FIG. 4F) increased. Indicative of the behavioral impact of skin-selective transgene expression, we observed significantly increased and robust spontaneous scratching 2 days post-induction of the B-raf transgene (FIG. 4G). Thus, TRPV4 activation in skin keratinocytes by LPC triggers intracellular MAP kinases pMEK and pERK, a significant event in pruritogenesis.

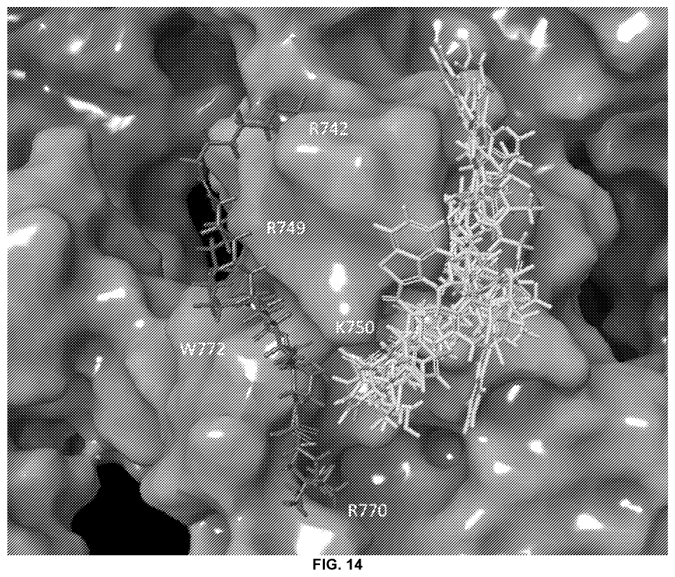

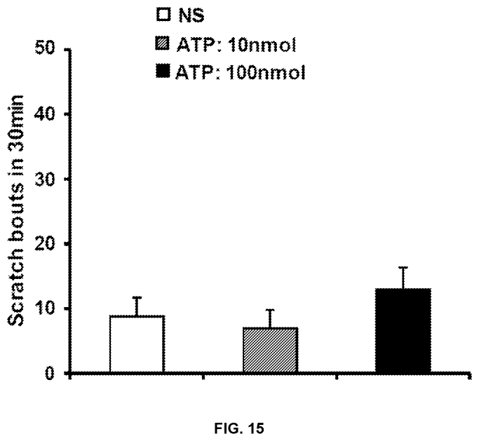

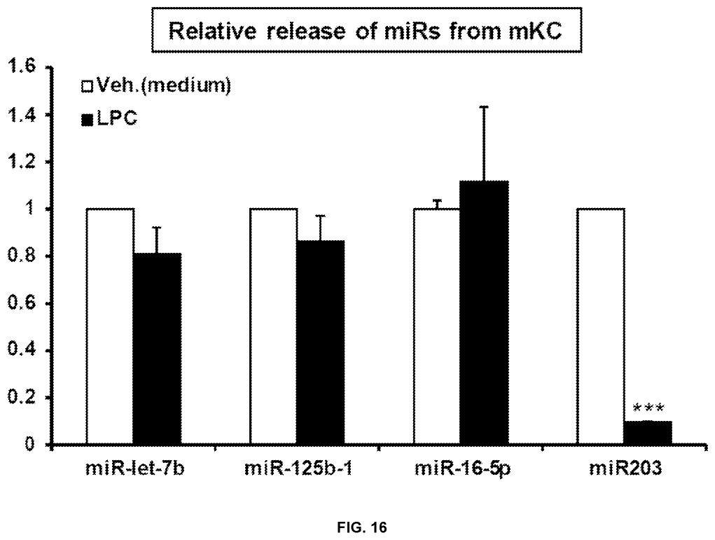

We then queried paracrine-secretory functions of keratinocytes that underlie activation of skin-innervating pruriceptor nerve endings. Given the TRPV4-dependence of release of ATP from epithelial cells and subsequent impact of ATP on sensory-neural function, we tested pruritogenicity of ATP and found it not to have any pruritogenic effects (FIG. 15). We then focused on secreted microRNAs (miRs) because these molecules can signal in a paracrine manner directly via cell surface receptors such as toll-like receptors or TRP ion channels (for example, TRPA1), which have been found expressed by peripheral sensory neurons and involved in itch and pain. We decided to test miR-let-7b, miR-125b-1, miR-16-5p, miR-203, and miR-146a because they are abundantly expressed in skin and involved in skin inflammation. We found keratinocytes' extracellular release of miR-let-7b, miR-125b-1, miR-16-5p, and miR-203 not to be increased in response to LPC (FIG. 16). However, we detected an increased release of miR-146a by LPC stimulation in both mouse and human keratinocytes (FIG. 4H-FIG. 4I). This increase was eliminated by pre-treatment with TRPV4 inhibitors GSK205 and HC067047 (FIG. 4H), and also by pretreatment with MEK-inhibitor U0126 (FIG. 4I). We interrogated miR-146a more because of its suspected inflammation-modulatory effects, and also because it was previously detected in skin and serum of pruritic atopic dermatitis and psoriasis. Our data indicated that LPC activates keratinocyte TRPV4, which then signals via MEK-ERK to trigger extracellular release of skin inflammation-associated miR-146a. Our results are in agreement with previous findings that inhibition of MEK/ERK suppresses vesicular biogenesis and secretion.

In order to elucidate mechanisms of extracellular release of miR-146a, we focused on Rab5a and Rab27a because: (1) Rab5a is important for early vesicle biogenesis; (2) Rab27a plays a critical role in docking of late endosomes to the plasma membrane; and (3) Rab5a and Rab27a are potential downstream targets of ERK in vesicular release in prostate and thyroid cancer cell lines. We isolated keratinocyte culture supernatant and measured acetylcholine esterase (AChE) activity to quantify the vesicle-bound fraction (Gupta et al. Am. J. Physiol. Heart Circ. Physiol. 2007, 292, H3052-3056; Malik et al. Methods Mol. Biol. 2016, 1448, 237-248). We noticed a moderate (˜10%) but significant increase of activity in keratinocyte supernatant in response to LPC at 15 min. This increase was completely eliminated by inhibition of pERK with U0126 or siRNA-knockdown of Rab5a and Rab27a (FIG. 4J-FIG. 4K). Extracellular-released miR-146a was significantly decreased (˜40%) when knocking down Rab5a and Rab27a (FIG. 4L). Decrease of vesicular release by only 10%, contrasting with decrease of extracellular miR-146a by 40%, when inhibiting MEK/ERK, Rab5a and Rab27a, and robust baseline extracellular release suggest active/ongoing release from keratinocytes that is increased only moderately upon TRPV4 activation by LPC. However, vesicular-fraction miR-146a increases appreciably, indicating that activation of keratinocyte-TRPV4 by LPC has a stronger impact on the released molecule.

### Example 6

### miR-146a is a Pruritogen and Functions Via TRPV1 in Primary Pruriceptor Neurons

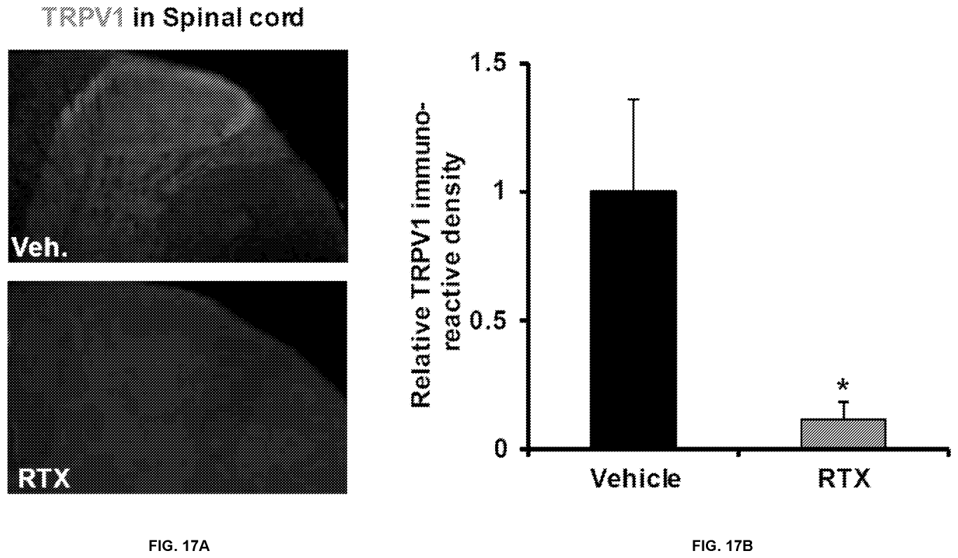

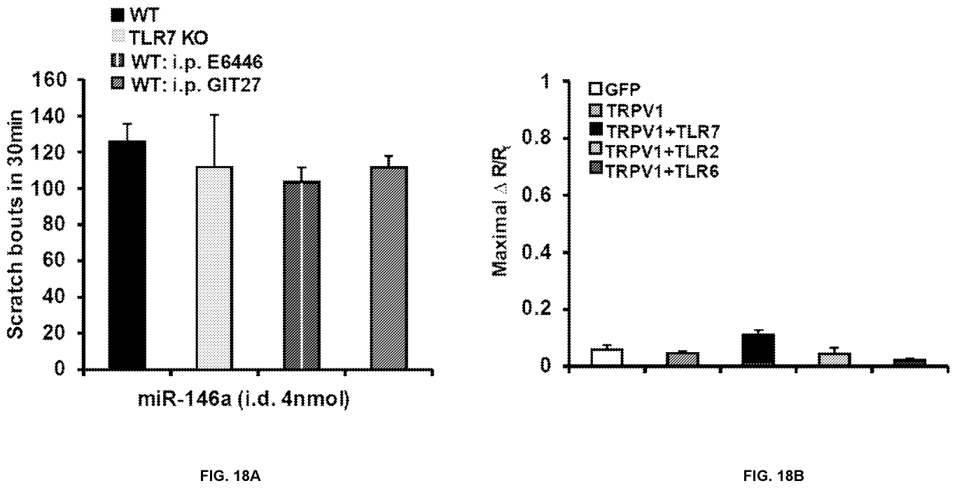

To address whether miR-146a is pruritic, we i.d. injected it into mice and observed dose-dependent scratching (FIG. 5A). This scratching response to miR-146a (4 nmol/50 μL) was immediate (latencies: 25 sec for miR-146a, vs 55 sec for LPC), indicating non-delayed signaling of miR-146a to skin-innervating peripheral pruriceptor axons as opposed to an indirect mechanism such as gene regulation, as known for microRNAs. Of note we used about 10 times less concentration of miR-146a than that of another pruritic micro-RNA miR-711. Additionally, using a mouse cheek model, we found i.d. injection of miR-146a induced scratching but not pain-related wiping behavior (FIG. 5B). We next tested whether miR-146a-induced itch relied on TRPV1+ pruriceptor neurons, given the established function of TRPV1 in pruriceptor peripheral neurons. Selective ablation of TRPV1⁺ central nerve terminals by i.t. injection of resiniferatoxin (RTX, FIG. 17A, FIG. 17B), an ultra-potent selective TRPV1 agonist, resulted in a significant reduction of scratching induced by miR-146a, similar for LPC (FIG. 5C). Thus the signaling chain LPC→keratinocyte TRPV4→extracellularly-released miR-146a relies on TRPV1⁺ pruriceptor neurons for causing itch.

We extended these findings by conducting the experiment in Trpv1 KO mice and in WT mice pre-treated with the TRPV1 inhibitor SB366791 which was applied i.p. or i.t. We observed that miR-146a-induced itch or LPC-induced itch was significantly reduced by knockout or inhibition of TRPV1 (FIG. 5D-FIG. 5E), indicating a reliance of pruritic effects of miR-146a and LPC on TRPV1. We next tested whether TRPA1, also established as pruriceptor channels, contributes to pruritogenicity of miR-146a or LPC. Our results were non-affirmative (FIG. 5F-FIG. 5G), indicating a lack of significant participation by TRPA1 in scratching induced by miR-146a or LPC. Notably, however, none of our TRPV1-targeting approaches completely eliminated scratching behavior, suggesting additional contributory signaling mechanisms.

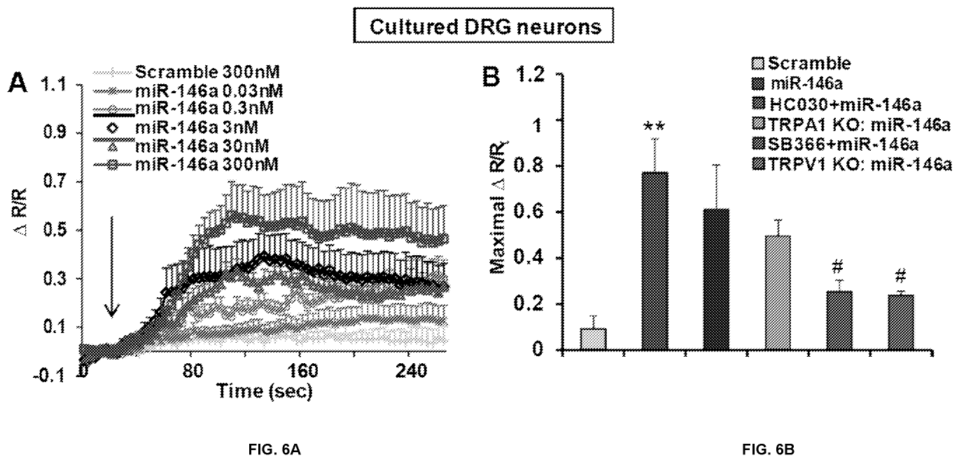

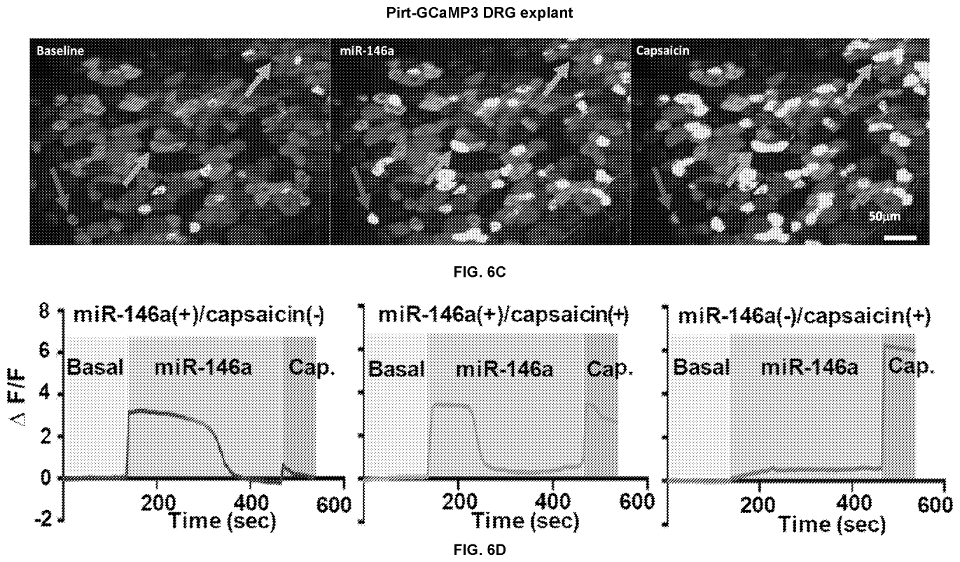

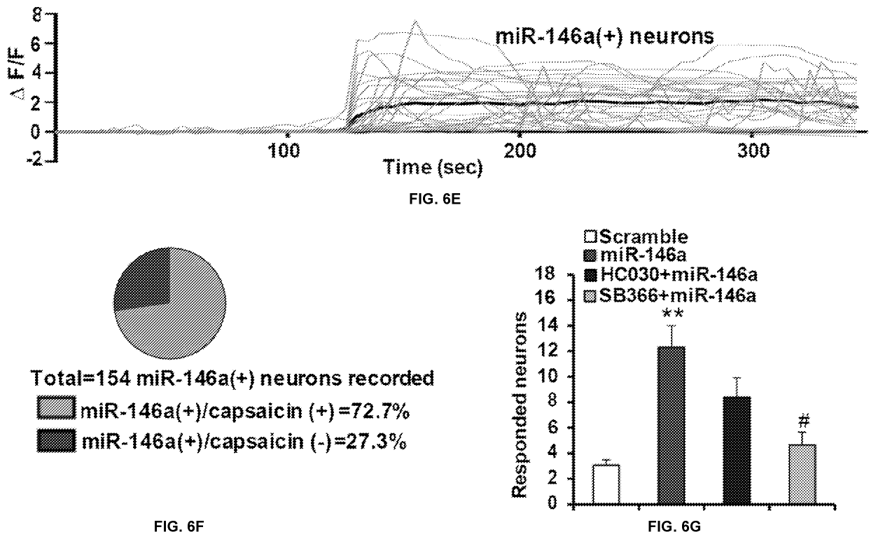

We next evaluated the cellular signaling of sensory neurons in response to miR-146a. In dissociated DRG neurons, we observed that miR-146a elicited Ca²⁺ influx in a dose-dependent manner (FIG. 6A). This signal was dependent on TRPV1 but not TRPA1, as observed when using selective inhibitors and dissociated neurons from Trpv1 KO and Trpa1 KO mice (FIG. 6B). We extended these findings to an organotypic preparation from live animals, in which the Ca²⁺ indicator GCaMP3, directed by the Pirt promoter, is expressed in DRG neurons. When ex-vivo DRG explants were stimulated with miR-146a, we recorded a direct Ca²⁺ transient in L4-5 DRG neurons (FIG. 6C-FIG. 6E). Responsive neurons made up ˜12% of DRG neurons (vs ˜3% for miR-146a scrambled control, FIG. 6G). Responder neurons were 72.7% capsaicin-responsive, the remainder capsaicin non responsive (FIG. 6F). The percentage of responder neurons was significantly reduced when inhibiting TRPV1, but not TRPA1 (FIG. 6G).

These findings support the novel concept that extracellularly-released miR-146a from skin keratinocytes in response to TRPV4 activation by LPC, activates skin-innervating TRPV1⁺ pruriceptor sensory neurons. Their peripheral epidermal projections are activated in a paracrine manner to elicit itch. Recent studies have demonstrated that extracellular miRs can either directly or indirectly via toll-like receptor 7 (TLR7) activate TRPA1 to elicit pain or itch and activation of TLR2/TLR6 heterodimers induces pain through TRPV1 and TRPA1. Here, we could not corroborate that miR-146a-induced itch was significantly influenced by knockout or inhibition of TLR7 or inhibition of TLR2/6 (FIG. 18A). In addition, we did not see significant Ca²⁺ influx upon stimulation with miR-146a in HEK293 cells transfected with rTRPV1 or co-transfected with rTRPV1 and rTLR7, 2, or 6 (FIG. 18B). These results fail to corroborate a critical role for TLR2, 6, and 7 functioning upstream of TRPV1 in response to miR-146a. Future studies may elucidate how miR-146a activates TRPV1⁺ pruriceptor neurons at increased mechanistic resolution.

### Example 7

### LPC and miR-146a are Elevated in Cholestatic Pruritic Mice and in PBC Patients with Cholestatic Itch

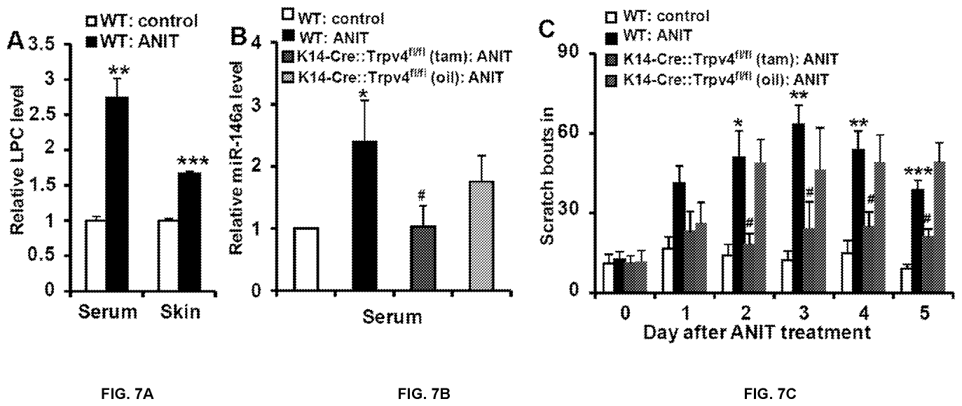

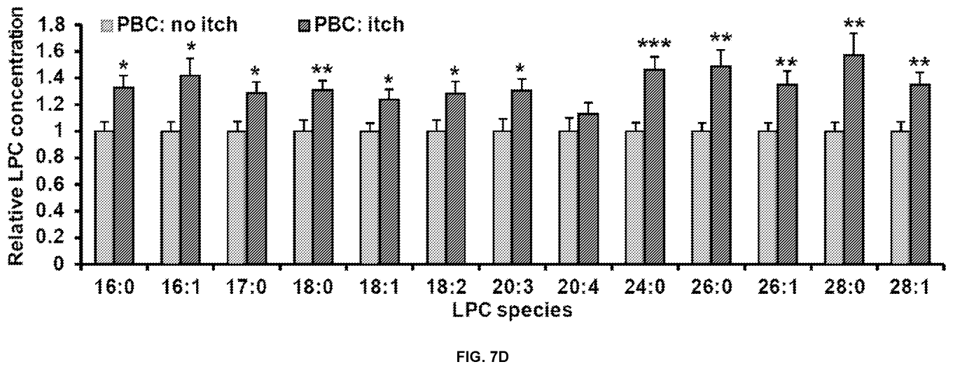

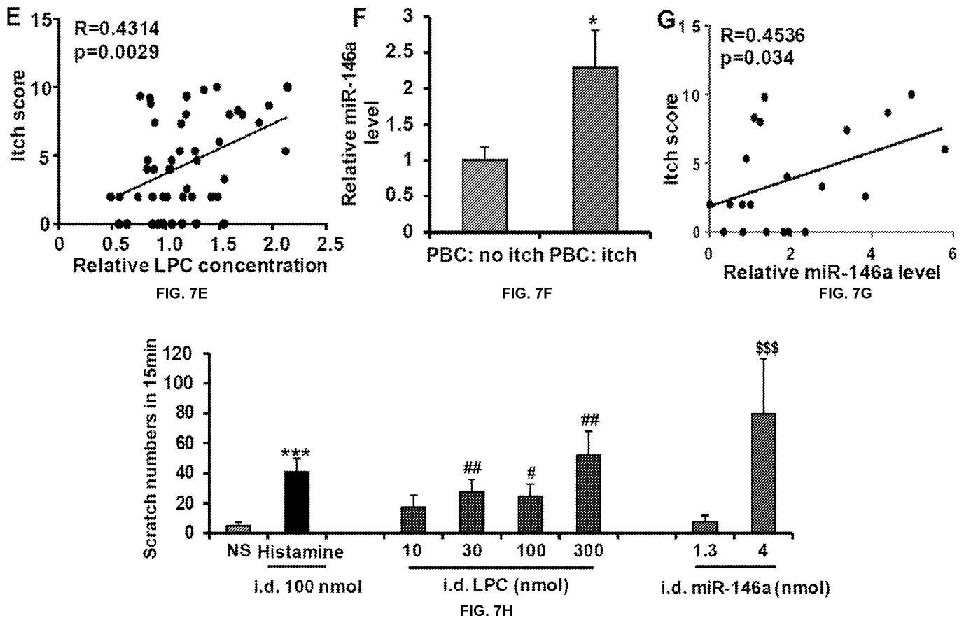

Next we addressed the role of keratinocyte-TRPV4 in itch and whether LPC and miR-146a function as pruritogens in a cholestasis disease-relevant context. We examined if levels of LPC and miR-146a are increased in a mouse model of cholestasis induced by systemic administration of α-naphthyl-isothiocyanate (ANIT). ANIT is a well-established preclinical model that elicits intrahepatic cholestasis and cholestasis-associated itch in mice. We detected a significant increase of LPC in both sera and skin (FIG. 7A), and elevated systemic miR-146a in ANIT-treated mice vs. controls (FIG. 7B). We next examined the contribution of keratinocyte-TRPV4 to cholestatic itch. ANIT-induced cholestatic itch (FIG. 7C) was almost eliminated in keratinocyte-Trpv4 cKO mice. This indicates a key role for keratinocytes and keratinocyte TRPV4 signaling in cholestatic itch. Moreover, systemic miR-146a was significantly reduced in keratinocyte-Trpv4 cKO ANIT-mice (FIG. 7B), indicating keratinocytes' release of miR-146a depended on cell-autonomous TRPV4 signaling.

We next measured LPC species in sera of patients with cholestatic itch, specifically in patients suffering from PBC. PBC represents the most prevalent immune-mediated cholestatic liver disease with many patients suffering gravely from pruritus. We detected a significant increase of the vast majority of LPC species in PBC patients with itch versus no-itch (FIG. 7D). Further analysis of itch intensity for individual patients revealed a significant correlation with LPC concentrations (R=0.4314, p<0.0029, FIG. 7E). We also measured systemic levels of miR-146a in pruritic PBC patients and found miR-146a significantly elevated and correlated with itch intensity (FIG. 7F-FIG. 7G). These findings, together with our other data, support the concept that systemic LPC and miR-146a upregulation contribute to pruritus in PBC. Furthermore, our findings raise the possibility that blood levels of LPC together with systemic abundance of miR-146a can serve as biomarkers for pruritus in PBC and possibly other cholestatic liver diseases.

### Example 8

### LPC and miR-146a Evoke Itch in Nonhuman Primates

Given our finding of elevated systemic LPC and miR-146a in pruritic PBC patients, together with our mechanistic insights in mice and human primary keratinocytes, we addressed whether LPC and miR-146a can also elicit pruritus in primates. Both molecules, upon i.d. injection, induced pruritus in rhesus monkeys in a dose-dependent manner (FIG. 7H); results were similar to those in mice. This suggested that our newly deconstructed pruritus mechanism in mice and human primary keratinocytes extends to primates, further supporting our premise that LPC and miR-146a contribute to cholestatic itch.

### Example 9

### Discussion

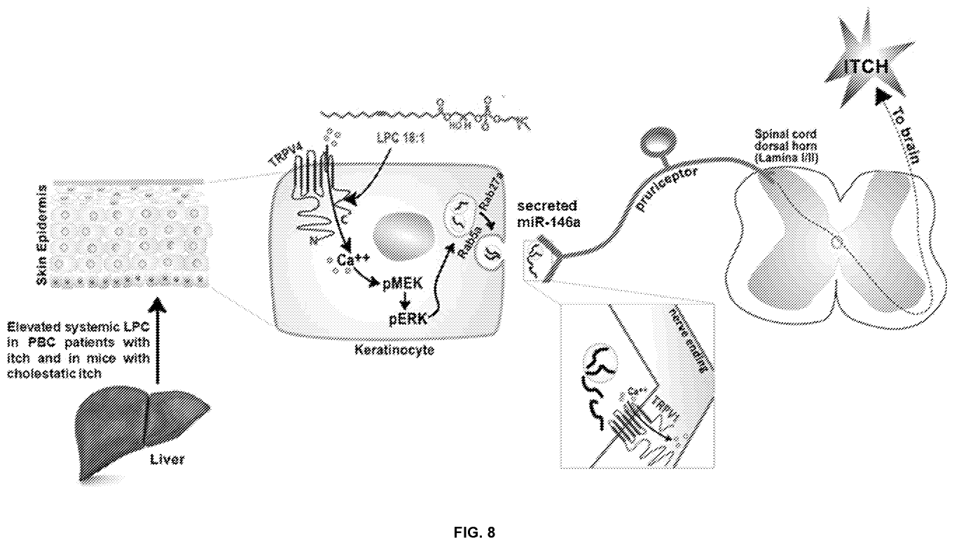

Here we describe a new signaling pathway of skin-sensory neuron crosstalk relevant for cholestatic itch (see FIG. 8). Our findings characterize the skin as a hitherto overlooked critical participant in cholestatic itch. Cholestatic itch has been viewed as the diseased liver generating pruritogenic mediators which then sensitize and activate pruriceptor sensory neurons to evoke perception of itch via neural transmission. Remarkably, this paradigm has not offered much as to (i) why the itch sensation originates from the epidermis, (ii) which molecular mechanism in skin cells are at play, and (iii) how skin cells communicate with pruriceptor neurons' peripheral axons in cholestatic itch. Our findings provide an initial answer, and we deconstructed “forefront” signaling involving epidermal keratinocytes responding to LPC via TRPV4 activation, then signaling by extracellular release of miR-146a to TRPV1⁺ skin-innervating peripheral nerve endings, activating these pruriceptors. We identified a novel pruritogen LPC, which is biosynthetically upstream of LPA, a previously-implicated pruritogen in cholestatic itch. Elevated LPC and miR-146a were detected systemically in PBC patients and mice with cholestatic itch. LPC and miR-146a were pruritic in mice and nonhuman primates. We observed that the pruritogenic effects of LPC and cholestatic itch in mice relied on keratinocyte-TRPV4. In addition, direct activation of TRPV4 by LPC led to Ca²⁺-influx into keratinocytes which triggered MEK-ERK MAP-kinase signaling, which in turn evoked extracellular release of miR-146a relying on Rab5a and Rab27a.

These findings have translational relevance for cholestatic itch in PBC, the most common immune-mediated liver disease, based on elevated LPC and miR-146a and their correlations with itch intensity in PBC patients, and supported by our findings in a preclinical mouse cholestasis model and a nonhuman primate itch model. Because LPC and miR-146a were elevated in the blood of PBC patients, the concept of LPC and miR-146a as possible biomarkers of cholestatic itch can now be addressed. Possibly our findings also apply to other hepatic pruritic diseases, another interesting subject for future studies. We note that elevated LPC was previously observed in patients with uremic pruritus, psoriasis, and atopic dermatitis. In regards to cholestatic itch, we emphasize that this condition is almost certainly not mono-factorial. Most likely other metabolites function in a co-contributory manner, namely bilirubin, bile acids, LPA. However, it is clear that (i) LPC is a robust pruritogen, (ii) LPC has its own unique pathway in itch, although conversion of LPC into LPA contributes to LPC pruritogenicity, (iii) systemic LPC concentrations are significantly elevated in pruritic PBC patients and correlated with itch intensity, and (iv) systemic and skin LPC concentrations are significantly elevated in cholestatic mice. Considering that systemic LPC, ATX, and LPA are increased in patients with cholestatic liver disease, one would argue that ATX would be a negative biomarker rather than a positive predictor of cholestatic itch because it would promote the conversion of potent pruritogen LPC into less potent pruritogen LPA. This may not be the case because LPC levels in human blood are ≥100× higher than those of LPA, suggesting that only a small fraction of LPC might be converted to LPA by ATX in-vivo. In that case, upregulation of ATX increases pruritus by increasing LPA, without significantly changing the concentration of LPC. On the other hand, inhibition of ATX would partially reduce LPC-caused itch because it attenuates LPC→LPA conversion. Previous findings and our own current data in ensemble suggest that elevated levels of LPC, ATX, and LPA can be concurrently present in an organism under pathologic conditions.

Our findings are significant for medical translation of sensory disorders like chronic itch because several molecules in the readily-targetable integument can be considered for clinical development, including TRPV4, TRPV1, MEK/ERK, and miR-146a. The results of pruritogenicity of LPC and miR-146a in primates are also relevant. Of note, a previous study in human volunteers demonstrated that i.d. injection of LPC caused histopathology of skin irritation and inflammation, but pruritogenicity was not tested. Decades before, localized allergic inflammation upon LPC injection into human skin was reported.

Another previous study on TRPV4 expression in chronic pruritus in humans is relevant. Chronic pruritus was associated with an increased expression of TRPV4 in the epidermis of subjects, and these patients had increased sensations to capsaicin, including pruritus, burning, and warmth. This finding, namely that TRPV4 gain-of-function in pruritic skin sensitizes TRPV1 signaling in sensory neurons, appears in agreement with our study.

Micro-RNAs are small, highly conserved, non-coding RNA molecules with known roles in RNA silencing and post-transcriptional regulation of gene expression. Their extracellular abundance has led to their use as biomarkers for diseases. Interestingly, recent work discovered an unconventional role for miRs: they can either directly or indirectly activate TRPA1 in sensory neurons to induce itch or pain. miR-146a has been found to be immunomodulatory in inflammation with postulated pro-resolution roles in psoriasis and atopic dermatitis, both of which are pruritic. Our experiments detailed herein support a new aspect of miR-146a in skin and sensory biology, namely that extracellular release of miR-146a from keratinocytes acts as ‘transmitter’ between keratinocytes and skin-innervating sensory neurons to primarily trigger itch. Indeed, i.d. injection of miR-146a elicited a more rapid scratching response (latency: ˜25 sec) than that of LPC (˜55 sec), suggesting that (i) miR-146a induces itch without affecting gene regulation, and (ii) it functions downstream of LPC in itch. In addition, miR-146a did not elicit pain behavior in the mouse cheek model, indicating its selective activation of neural itch pathways. The mechanism of how miR-146a activates TRPV1⁺ sensory neurons may be further studied, especially in terms of miR-146a evoking itch, not pain.

We discovered a new binding site for LPC (18:1) in the C-terminus of TRPV4, directly adjacent to the TRP-helix. The widely-used synthetic activator GSK101 does not bind here and activates heterologously-expressed TRPV4 with higher single channel conductance than the natural activator, LPC (18:1). Indeed, our discovery defines the first endogenous glycerophospholipid activator of TRPV4. Given that GSK101 is lethal in-vivo, the quest for a therapeutically-beneficial (for example, in arthritis, hepatic or renal disease) TRPV4 activator continues. Our discovery of the LPC (18:1)-TRPV4 binding site provides a framework for development of non-lethal TRPV4-activating molecules and inverse agonists. Also, TRPV4's gating mechanism can now be interrogated by comparing LPC (18:1) bound to TRPV4 vs GSK101 bound to TRPV4 using structural methods.

We view our discovery as a novel concept in the sensory submodality of cholestatic itch. We identified a hitherto non-recognized glycerophospholipid, LPC, as a pruritogen that initiates the signaling cascade in the skin, plus the messenger of the epithelia-sensory neuron crosstalk, an immunomodulatory micro-RNA, miR-146a, that directly activates the pruriceptor sensory neurons. TRPV4 on keratinocytes and TRPV1 on pruriceptor sensory neurons function synergistically as molecular players in this debilitating form of itch.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A method of treating a subject having an itch-related disorder, the method comprising: determining the level of a biomarker in a biological sample from the subject, wherein the biomarker is selected from TRPV4 expression, lysophosphatidylcholine, and miRNA-146a expression, or a combination thereof; and administering an anti-pruritic therapy to treat the subject identified as having the itch-related disorder, wherein the subject is identified as having the itch-related disorder when the level of the biomarker is greater in the biological sample than in a control sample.

Clause 2. A method of treating an itch-related disorder in a subject, the method comprising: (a) determining the level of a biomarker in a biological sample from the subject, wherein the biomarker is selected from TRPV4 expression, lysophosphatidylcholine, and miRNA-146a expression, or a combination thereof, and wherein the level of the biomarker is greater in the biological sample than in a control sample; (b) diagnosing the subject as having an itch-related disorder based on the level of the biomarker determined in step (a); and (c) administering an anti-pruritic therapy to the subject diagnosed as having an itch-related disorder in step (b).

Clause 3. A method of diagnosing an itch-related disorder in a subject, the method comprising: determining the level of a biomarker in a biological sample from the subject, wherein the biomarker is selected from TRPV4 expression, lysophosphatidylcholine, and miRNA-146a expression, or a combination thereof; and diagnosing the subject as having an itch-related disorder when the level of the biomarker is greater in the biological sample than in a control sample.

Clause 4. The method of clause 3, wherein the method further comprises administering an anti-pruritic therapy to the subject diagnosed as having an itch-related disorder.

Clause 5. The method of any one of clauses 1-4, wherein the itch-related disorder comprises itch.

Clause 6. A method of identifying an itch-related disorder in a subject, the method comprising: (i) obtaining a biological sample from the subject; (ii) identifying the presence of a biomarker in the subject, the biomarker selected from the group consisting of TRPV4, miRNA-146a, lysophosphatidylcholine, and combinations thereof; (iii) quantifying the expression level of the biological sample, in which the presence of one or more of the biomarkers in an amount greater than the control is indicative of the itch-related disorder comprising itch; and (iv) administering to the subject an appropriate anti-pruritic therapy if the level of biomarker is greater in the biological sample than in a control sample.

Clause 7. The method of any one of clauses 1-6, wherein the biomarker is the level of TRPV4 expression.

Clause 8. The method of any one of clauses 1-6, wherein the biomarker is the level of lysophosphatidylcholine.

Clause 9. The method of any one of clauses 1-6, wherein the biomarker is the level of miRNA-146a expression.

Clause 10. The method of any one of clauses 1-9, wherein the itch-related disorder is a dermatological disorder or a systemic disorder.

Clause 11. The method of clause 10, wherein the itch-related disorder is a systemic disorder selected from liver disorder, kidney disorder, cancer, lymphoma, infection, or medication side-effect.

Clause 12. The method of any one of clauses 1-9, wherein the itch-related disorder is selected from the group consisting of cholestatic itch, uremic itch, pruritic psoriasis, and combinations thereof.

Clause 13. The method of any one of clauses 1-12, wherein the level of TRPV4 expression or the level of miRNA-146a expression is an RNA expression level.

Clause 14. The method of any one of clauses 1-13, wherein the level of the biomarker is determined by microarray analysis, or PCR, or a combination thereof.

Clause 15. The method of any one of clauses 1-14, wherein the control sample is from a healthy subject.

Clause 16. The method of any one of clauses 1-15, wherein the biological sample comprises skin.

Clause 17. The method of clause 16, wherein the biological sample comprises skin keratinocytes.

Clause 18. The method of any one of clauses 1-15, wherein the biological sample comprises blood.

Clause 19. The method of any one of clauses 1-2 and 4-18, wherein the anti-pruritic therapy is selected from the group consisting of moisturizers, capsaicin, salicylic acid, emollients, topical corticosteroids, topical calcineurin inhibitors, antihistamines, menthol, local anesthetics, cannabinoids, immunomodulators, antihistamines, antidepressants, μ-opiod receptor agonists, k-opiod receptor agonists, neuroleptics, substance P antagonist, immunosuppressants, methylnaltrexone, NGX-4010, TS-022, Serine proteases/PAR2 antagonists, IL-31 antibody, IL-4-receptor antibody, IL-13 antibody, TSLP-antibody, IL-5 antibody, and combinations thereof.

Clause 20. The method of clause 19, wherein the anti-pruritic therapy comprises an immunomodulator.

Clause 21. The method of clause 20, wherein the immunomodulator comprises a TRPV4 inhibitor.

Clause 22. The method of any one of clauses 1-21, wherein the subject is a mammal.

Clause 23. The method of any one of clauses 1-2 and 4-22, wherein the anti-pruritic therapy comprises a TRPV4 inhibitor.

Clause 24. The method of clause 23, wherein the TRPV4 inhibitor binds to a C-terminal region of TRPV4.

Clause 25. The method of clause 24, wherein the TRPV4 inhibitor binds at least one amino acid in a motif comprising K750-W772 of Xenopus TRPV4 or K754-W776 of mammalian TRPV4 or an amino acid corresponding thereto.

Clause 26. The method of clause 24, wherein the TRPV4 inhibitor binds at least one amino acid in a motif comprising R742-W772 of Xenopus TRPV4 or R746-W776 of mammalian TRPV4 or an amino acid corresponding thereto.

Clause 27. The method of clause 24, wherein the TRPV4 inhibitor binds at least one amino acid in a motif comprising K750-W772 and R742 of Xenopus TRPV4 or K754-W776 and R746 of mammalian TRPV4 or an amino acid corresponding thereto.

Clause 28. The method of clause 24, wherein the TRPV4 inhibitor binds Arg-746 of mammalian TRPV4 or Arg-742 of Xenopus TRPV4 or an amino acid corresponding thereto.

Clause 29. The method of clause 24, wherein the TRPV4 inhibitor binds at least one amino acid selected from K754, R757, R774, and W776 of mammalian TRPV4 or an amino acid corresponding thereto.

Clause 30. A method of screening for a compound that modulates TRPV4, the method comprising: testing a plurality of compounds for binding to wild-type TRPV4 to determine from the plurality of compounds a subset of compounds that bind wild-type TRPV4; and testing the subset of compounds that bind wild-type TRPV4 for binding to at least one mutant TRPV4, wherein the mutant TRPV4 comprises a mutation of at least one amino acid in the motif corresponding to K746-W776 of mammalian TRPV4, or an amino acid corresponding thereto, to determine from the subset of compounds a compound that binds wild-type TRPV4 but not the mutant TRPV4.

Clause 31. The method of clause 30, wherein at least one amino acid in the motif corresponding to K754-W776 of mammalian TRPV4 is mutated to an alanine.

Clause 32. The method of clause 30, wherein at least one amino acid in the motif corresponding to K754-W776 of mammalian TRPV4 is mutated to a glycine.

Clause 33. The method of clause 30, wherein at least one amino acid selected from K754, R757, R774, and W776 of mammalian TRPV4, or an amino acid corresponding thereto, is mutated.

Clause 34. The method of any one of clauses 30-33, wherein the mutant TRPV4 has activity as an ion channel.

Clause 35. The method of any one of clauses 30-34, further comprising determining the effect of the compound that binds wild-type TRPV4 but not the mutant TRPV4 on the activity of wild-type TRPV4.

Clause 36. The method of any one of clauses 30-35, wherein the compound that binds wild-type TRPV4 but not the mutant TRPV4 inhibits the activity of wild-type TRPV4.

Clause 37. The method of any one of clauses 30-35, wherein the compound that binds wild-type TRPV4 but not the mutant TRPV4 increases the activity of wild-type TRPV4.

Clause 38. The method of any one of clauses 30-35, wherein the compound that binds wild-type TRPV4 but not the mutant TRPV4 inhibits or reduces the binding of LCP to wild-type TRPV4.

SEQUENCES
TRPV4 protein, human variant 1 (Accession No. NM_021625)
SEQ ID NO: 1
MADSSEGPRAGPGEVAELPGDESGTPGGEAFPLSSLANLFEGEDGSLSPSPADASRPAGPGD
GRPNLRMKFQGAFRKGVPNPIDLLESTLYESSVVPGPKKAPMDSLFDYGTYRHHSSDNKRWR
KKIIEKQPQSPKAPAPQPPPILKVENRPILFDIVSRGSTADLDGLLPFLLTHKKRLTDEEFR
EPSTGKTCLPKALLNLSNGRNDTIPVLLDIAERTGNMREFINSPERDIYYRGQTALHIAIER
RCKHYVELLVAQGADVHAQARGRFFQPKDEGGYFYFGELPLSLAACTNQPHIVNYLTENPHK
KADMRRQDSRGNTVLHALVAIADNTRENTKFVTKMYDLLLLKCARLFPDSNLEAVLNNDGLS
PLMMAAKTGKIGIFQHIIRREVTDEDTRHLSRKFKDWAYGPVYSSLYDLSSLDTCGEEASVL
EILVYNSKIENRHEMLAVEPINELLRDKWRKFGAVSFYINVVSYLCAMVIFTLTAYYQPLEG
TPPYPYRTTVDYLRLAGEVITLFTGVLFFFTNIKDLFMKKCPGVNSLFIDGSFQLLYFIYSV
LVIVSAALYLAGIEAYLAVMVFALVLGWMNALYFTRGLKLTGTYSIMIQKILFKDLERFLLV
YLLFMIGYASALVSLLNPCANMKVCNEDQTNCTVPTYPSCRDSETFSTFLLDLFKLTIGMGD
LEMLSSTKYPVVFIILLVTYIILTFVLLLNMLIALMGETVGQVSKESKHIWKLQWATTILDI
ERSFPVFLRKAFRSGEMVTVGKSSDGTPDRRWCFRVDEVNWSHWNQNLGIINEDPGKNETYQ
YYGFSHTVGRLRRDRWSSVVPRVVELNKNSNPDEVVVPLDSMGNPRCDGHQQGYPRKWRTDD
APL
TRPV4 DNA, human variant 1
SEQ ID NO: 2
attcaggaag cgcggatctc ccggccgccg gcgcccagcc gtcccggagg ctgagcagtg
cagacgggcc tggggcaggc atggcggatt ccagcgaagg cccccgcgcg gggcccgggg
aggtggctga gctccccggg gatgagagtg gcaccccagg tggggaggct tttcctctct
cctccctggc caatctgttt gagggggagg atggctccct ttcgccctca ccggctgatg
ccagtcgccc tgctggccca ggcgatgggc gaccaaatct gcgcatgaag ttccagggcg
ccttccgcaa gggggtgccc aaccccatcg atctgctgga gtccacccta tatgagtcct
cggtggtgcc tgggcccaag aaagcaccca tggactcact gtttgactac ggcacctatc
gtcaccactc cagtgacaac aagaggtgga ggaagaagat catagagaag cagccgcaga
gccccaaagc ccctgcccct cagccgcccc ccatcctcaa agtcttcaac cggcctatcc
tctttgacat cgtgtcccgg ggctccactg ctgacctgga cgggctgctc ccattcttgc
tgacccacaa gaaacgccta actgatgagg agtttcgaga gccatctacg gggaagacct
gcctgcccaa ggccttgctg aacctgagca atggccgcaa cgacaccatc cctgtgctgc
tggacatcgc ggagcgcacc ggcaacatga gggagttcat taactcgccc ttccgtgaca
tctactatcg aggtcagaca gccctgcaca tcgccattga gcgtcgctgc aaacactacg
tggaacttct cgtggcccag ggagctgatg tccacgccca ggcccgtggg cgcttcttcc
agcccaagga tgaggggggc tacttctact ttggggagct gcccctgtcg ctggctgcct
gcaccaacca gccccacatt gtcaactacc tgacggagaa cccccacaag aaggcggaca
tgcggcgcca ggactcgcga ggcaacacag tgctgcatgc gctggtggcc attgctgaca
acacccgtga gaacaccaag tttgttacca agatgtacga cctgctgctg ctcaagtgtg
cccgcctctt ccccgacagc aacctggagg ccgtgctcaa caacgacggc ctctcgcccc
tcatgatggc tgccaagacg ggcaagattg ggatctttca gcacatcatc cggcgggagg
tgacggatga ggacacacgg cacctgtccc gcaagttcaa ggactgggcc tatgggccag
tgtattcctc gctttatgac ctctcctccc tggacacgtg tggggaagag gcctccgtgc
tggagatcct ggtgtacaac agcaagattg agaaccgcca cgagatgctg gctgtggagc
ccatcaatga actgctgcgg gacaagtggc gcaagttcgg ggccgtctcc ttctacatca
acgtggtctc ctacctgtgt gccatggtca tcttcactct caccgcctac taccagccgc
tggagggcac accgccgtac ccttaccgca ccacggtgga ctacctgcgg ctggctggcg
aggtcattac gctcttcact ggggtcctgt tcttcttcac caacatcaaa gacttgttca
tgaagaaatg ccctggagtg aattctctct tcattgatgg ctccttccag ctgctctact
tcatctactc tgtcctggtg atcgtctcag cagccctcta cctggcaggg atcgaggcct
acctggccgt gatggtcttt gccctggtcc tgggctggat gaatgccctt tacttcaccc
gtgggctgaa gctgacgggg acctatagca tcatgatcca gaagattctc ttcaaggacc
ttttccgatt cctgctcgtc tacttgctct tcatgatcgg ctacgcttca gccctggtct
ccctcctgaa cccgtgtgcc aacatgaagg tgtgcaatga ggaccagacc aactgcacag
tgcccactta cccctcgtgc cgtgacagcg agaccttcag caccttcctc ctggacctgt
ttaagctgac catcggcatg ggcgacctgg agatgctgag cagcaccaag taccccgtgg
tcttcatcat cctgctggtg acctacatca tcctcacctt tgtgctgctc ctcaacatgc
tcattgccct catgggcgag acagtgggcc aggtctccaa ggagagcaag cacatctgga
agctgcagtg ggccaccacc atcctggaca ttgagcgctc cttccccgta ttcctgagga
aggccttccg ctctggggag atggtcaccg tgggcaagag ctcggacggc actcctgacc
gcaggtggtg cttcagggtg gatgaggtga actggtctca ctggaaccag aacttgggca
tcatcaacga ggacccgggc aagaatgaga cctaccagta ttatggcttc tcgcataccg
tgggccgcct ccgcagggat cgctggtcct cggtggtacc ccgcgtggtg gaactgaaca
agaactcgaa cccggacgag gtggtggtgc ctctggacag catggggaac ccccgctgcg
atggccacca gcagggttac ccccgcaagt ggaggactga tgacgccccg ctctagggac
tgcagcccag ccccagcttc tctgcccact catttctagt ccagccgcat ttcagcagtg
ccttctgggg tgtcccccca caccctgctt tggccccaga ggcgagggac cagtggaggt
gccagggagg ccccaggacc ctgtggtccc ctggctctgc ctccccaccc tggggtgggg
gctcccggcc acctgtcttg ctcctatgga gtcacataag ccaacgccag agcccctcca
cctcaggccc cagcccctgc ctctccatta tttatttgct ctgctctcag gaagcgacgt
gacccctgcc ccagctggaa cctggcagag gccttaggac cccgttccaa gtgcactgcc
cggccaagcc ccagcctcag cctgcgcctg agctgcatgc gccaccattt ttggcagcgt
ggcagctttg caaggggctg gggccctcgg cgtggggcca tgccttctgt gtgttctgta
gtgtctggga tttgccggtg ctcaataaat gtttattcat tgacggtg
miRNA-146a (Accession No. NR_029701)
SEQ ID NO: 3
ccgatgtgta tcctcagctt tgagaactga attccatggg ttgtgtcagt gtcagacctc
tgaaattcag ttcttcagct gggatatctc tgtcatcgt
stem-loop oligonucleotide specific for miR-146a-5p
SEQ ID NO: 4
GAGAACTGAATTCCATGGG
stem-loop oligonucleotide specific for miR-let-7b
SEQ ID NO: 5
GAGGTAGTAGGTTGTGTGG
stem-loop oligonucleotide specific for miR-125b-1
SEQ ID NO: 6
CCCT GAGACCCTAACTTG
stem-loop oligonucleotide specific for miR-203
SEQ ID NO: 7
GTGGTTC TTGACAGTTCAAC
stem-loop oligonucleotide specific for miR-16-5p
SEQ ID NO: 8
AGCAGCAC GTAAATATTGGC
Rab5a siRNA
(SEQ ID NO: 9)
5′-GUAGAAUCAA GUUUCUAAUUCUGAA-3′
(SEQ ID NO: 10)
5′-UUCAGAAUUAGAAACUUGAUUCUACCA-3′
Rab27a siRNA
(SEQ ID NO: 11)
5′-AGCUAAAA CUGAGAGCUUCAAACAG-3′
(SEQ ID NO: 12)
5′-CUGUUUGAAGCUCUCAGUUUUAGCUUA-3′
internal control β-tubulin primers
(SEQ ID NO: 13)
forward: 5′-CCTG CCTTTTCGTCTCTAGC CGC-3′
(SEQ ID NO: 14)
reverse: 5′-GCTGATGACCTCCCA GAACTTGGC-3′
TRPV4 primers
(SEQ ID NO: 15)
forward: 5′-GTGGGCAAGAGCTCAGATGGCACTC-3′
(SEQ ID NO: 16)
reverse: 5′-CCACCGAGG ACCAACGATCCCTAC G-3′
TRPV4, rat, protein (Accession No. NM_023970)
SEQ ID NO: 17
MADPGDGPRAAPGDVAEPPGDESGTSGGEAFPLSSLANLFEGEEGSSSLSPVDASRPAGPGD
GRPNLRMKFQGAFRKGVPNPIDLLESTLYESSVVPGPKKAPMDSLFDYGTYRHHPSDNKRWR
RKVVEKQPQSPKAPAPQPPPILKVFNRPILFDIVSRGSTADLDGLLSYLLTHKKRLTDEEFR
EPSTGKTCLPKALLNLSNGRNDTIPVLLDIAERTGNMREFINSPFRDIYYRGQTALHIAIER
RCKHYVELLVAQGADVHAQARGRFFQPKDEGGYFYFGELPLSLAACTNQPHIVNYLTENPHK
KADMRRQDSRGNTVLHALVAIADNTRENTKFVTKMYDLLLLKCSRLFPDSNLETVLNNDGLS
PLMMAAKTGKIGVFQHIIRREVTDEDTRHLSRKFKDWAYGPVYSSLYDLSSLDTCGEEVSVL
EILVYNSKIENRHEMLAVEPINELLRDKWRKFGAVSFYINVVSYLCAMVIFTLTAYYQPLEG
TPPYPYRTTVDYLRLAGEVITLLTGVLFFFTSIKDLEMKKCPGVNSLFVDGSFQLLYFIYSV
LVVVSAALYLAGIEAYLAVMVFALVLGWMNALYFTRGLKLTGTYSIMIQKILFKDLFRFLLV
YLLFMIGYASALVTLLNPCTNMKVCNEDQSNCTVPSYPACRDSETFSAFLLDLFKLTIGMGD
LEMLSSAKYPVVFILLLVTYIILTFVLLLNMLIALMGETVGQVSKESKHIWKLQWATTILDI
ERSEPVELR [symbol] AF [symbol] SGEMVTVGKSSDGTPD [symbol] R [symbol] CFRVDEVNWSHWNQNLGIINEDPGKSEIYQ
YYGFSHTMGRLRRDRWSSVVPRVVELNKNSGTDEVVVPLDNLGNPNCDGHQQGYAPKWRAED
APL
TRPV4, rat, DNA
SEQ ID NO: 18
gggaggagga cgcggcggga tcaggaagcg gctgcgctgc gcccgcgtcc caagcaggcc
gagaagtcca aacagatctg ctcagggtcc agtatggcag atcctggtga tggcccccgt
gcagcgcctg gggatgtggc tgagccccct ggagacgaga gtggcacttc tggtggggag
gccttccccc tctcttccct ggccaacctg tttgagggag aggaaggctc ctcttctctt
tcaccagtgg atgctagccg ccctgctggc cccggggatg gacgtccaaa cctgcgtatg
aagttccagg gcgctttccg caagggggtt cccaacccca ttgacctgct ggagtccacc
ctgtatgagt cctcagtagt gcctgggccc aagaaagcgc ccatggattc gttgttcgac
tatggcactt accggcacca ccccagtgac aacaagagat ggaggaggaa ggtcgtagag
aagcagccac agagccccaa agctcccgcc ccccagccac cccccatcct caaagtcttc
aaccggccca tcctctttga catcgtgtcc cggggctcca ctgccgacct ggacggactg
ctctcctact tgctgaccca caagaagcgc ctgactgatg aggagttccg ggaaccatcc
acagggaaga cctgcctgcc caaggcactt ctgaacttaa gcaatggccg aaacgacacc
atcccagtgt tgctggacat tgcggaacgc acgggcaaca tgcgggagtt catcaactcg
cccttcagag acatctacta ccgagggcag acggcactgc acatcgccat tgaacggcgc
tgcaagcatt acgtggagct cctggtggcc cagggagccg atgtgcacgc gcaggcccga
gggcggttct tccagcccaa ggatgagggt ggctacttct actttgggga gctgcccttg
tccttggcag cctgcaccaa ccagccgcac atcgtcaact acctgacaga gaaccctcac
aagaaagccg atatgaggcg acaggactcc agaggcaaca cggtgctcca cgcgctggtg
gccatcgctg acaacacccg agagaacacc aagtttgtca ccaagatgta tgacctgttg
cttctcaagt gctcccgcct cttcccagac agcaacctgg agactgtgct taacaatgac
ggtctttcgc ccctcatgat ggctgccaag actggcaaga tcggggtctt tcagcacatc
atccgacggg aggtgacaga tgaggacaca cggcacctgt ctcgcaagtt caaggactgg
gcctacgggc ctgtgtattc ttctctctac gacctctcct ccctggatac gtgcggggag
gaagtgtccg tgctggagat cctggtttac aacagcaaga tcgagaaccg ccatgagatg
ctggctgtgg agcccattaa cgaactgctg agggacaagt ggcgtaagtt cggggccgtg
tccttctaca tcaacgttgt ctcctatctg tgtgccatgg tcatcttcac cctcacagcc
tactatcagc cactggaggg cacgccaccc tacccttacc gtaccacggt ggactacctg
aggctggctg gtgaggtcat cacgctcctc acaggagtcc tgttcttctt taccagtatc
aaagacttgt tcatgaagaa atgccctgga gtgaattctc tcttcgtcga tggctccttc
cagttgctct acttcatcta ctcagtgctg gtggttgtgt ctgcggcgct ctacctggca
gggatcgagg cctatctggc tgtgatggtc tttgccctgg tcctgggctg gatgaatgcc
ctttacttca cccgtgggct gaagctgaca gggacctaca gcatcatgat tcagaagatc
ctcttcaaag atctcttccg ctttctgctg gtctacctgc tttttatgat tggctatgcc
tcagctctgg tcaccctcct gaatccgtgc accaacatga aggtctgtaa cgaggaccag
agcaactgca cggtgccctc ataccccgcg tgccgggaca gcgagacctt cagcgccttc
ctactggacc tcttcaagct caccatcggc atgggcgacc tggagatgct gagcagcgct
aagtaccccg tggtcttcat tctcctgctg gttacctaca tcatcctcac cttcgtgctc
ctgctgaaca tgctcatcgc cctcatgggt gagaccgtgg gccaggtgtc caaggagagc
aagcacatct ggaagctgca gtgggccacc accatcctgg acatcgagcg ctccttccct
gtgttcctga ggaaggcctt ccgctccgga gagatggtga cagtgggcaa gagctcggat
ggcactccag accgcaggtg gtgcttcagg gtggacgagg tgaactggtc tcactggaac
cagaacctgg gcatcattaa cgaggacccc ggcaagagcg agatctacca gtactatggc
ttctcccata ccatggggcg cctccgcagg gatcgctggt cctcagtggt gccccgcgtg
gtggagctga acaagaactc aggcacagat gaagtggtgg tccccctgga taacctaggg
aaccccaact gtgacggcca ccagcaaggt tatgctccca agtggagggc ggaggacgca
ccactgtagg ggccatgcca gggctggggt caatggccca ggcttggccc ttgctcccac
ctacatttca gcatctgtcc tgtgtcttcc cacacccaca cgtgacctcg gaggtgaggg
cctctgtgga gactctgggg aggccccagg accctctggt ccccacaaag acttttgctc
ttatttctac tcctccccac atgggggacg gggctcctgg ccacctgtct cactcccatg
gagtcaccta agccagctca gggcccctcc actcacaggg ctcaggcccc tgtccctctt
gtgcactatt tattgctctc ctcaggaaaa tgacatcaca ggagtctacc tgcagctgga
acctggccag ggctgaggct catgcaggga cactgcagcc ctgacccgct gcagatctga
cctgctgcag cccgggctag ggtgggtctt ctgtactttg tagagatcgg ggctgttggt
gctcaataaa tgtttgttta ttctcggtgg a

1. A method of treating a subject having an itch-related disorder, the method comprising:
determining the level of a biomarker in a biological sample from the subject, wherein the biomarker is selected from TRPV4 expression, lysophosphatidylcholine, and miRNA-146a expression, or a combination thereof; and
administering an anti-pruritic therapy to treat the subject identified as having the itch-related disorder, wherein the subject is identified as having the itch-related disorder when the level of the biomarker is greater in the biological sample than in a control sample.

2. A method of treating an itch-related disorder in a subject, the method comprising:
(a) determining the level of a biomarker in a biological sample from the subject, wherein the biomarker is selected from TRPV4 expression, lysophosphatidylcholine, and miRNA-146a expression, or a combination thereof, and wherein the level of the biomarker is greater in the biological sample than in a control sample;
(b) diagnosing the subject as having an itch-related disorder based on the level of the biomarker determined in step (a); and
(c) administering an anti-pruritic therapy to the subject diagnosed as having an itch-related disorder in step (b).

3. A method of diagnosing an itch-related disorder in a subject, the method comprising:
determining the level of a biomarker in a biological sample from the subject, wherein the biomarker is selected from TRPV4 expression, lysophosphatidylcholine, and miRNA-146a expression, or a combination thereof; and
diagnosing the subject as having an itch-related disorder when the level of the biomarker is greater in the biological sample than in a control sample.

4. The method of claim 3, wherein the method further comprises administering an anti-pruritic therapy to the subject diagnosed as having an itch-related disorder.

5. The method of any one of claims 1-4, wherein the itch-related disorder comprises itch.

6. A method of identifying an itch-related disorder in a subject, the method comprising:
(i) obtaining a biological sample from the subject;
(ii) identifying the presence of a biomarker in the subject, the biomarker selected from the group consisting of TRPV4, miRNA-146a, lysophosphatidylcholine, and combinations thereof;
(iii) quantifying the expression level of the biological sample, in which the presence of one or more of the biomarkers in an amount greater than the control is indicative of the itch-related disorder comprising itch; and
(iv) administering to the subject an appropriate anti-pruritic therapy if the level of biomarker is greater in the biological sample than in a control sample.

7. The method of any one of claims 1-6, wherein the biomarker is the level of TRPV4 expression.

8. The method of any one of claims 1-6, wherein the biomarker is the level of lysophosphatidylcholine.

9. The method of any one of claims 1-6, wherein the biomarker is the level of miRNA-146a expression.

10. The method of any one of claims 1-9, wherein the itch-related disorder is a dermatological disorder or a systemic disorder.

11. The method of claim 10, wherein the itch-related disorder is a systemic disorder selected from liver disorder, kidney disorder, cancer, lymphoma, infection, or medication side-effect.

12. The method of any one of claims 1-9, wherein the itch-related disorder is selected from the group consisting of cholestatic itch, uremic itch, pruritic psoriasis, and combinations thereof.

13. The method of any one of claims 1-12, wherein the level of TRPV4 expression or the level of miRNA-146a expression is an RNA expression level.

14. The method of any one of claims 1-13, wherein the level of the biomarker is determined by microarray analysis, or PCR, or a combination thereof.

15. The method of any one of claims 1-14, wherein the control sample is from a healthy subject.

16. The method of any one of claims 1-15, wherein the biological sample comprises skin.

17. The method of claim 16, wherein the biological sample comprises skin keratinocytes.

18. The method of any one of claims 1-15, wherein the biological sample comprises blood.

19. The method of any one of claims 1-2 and 4-18, wherein the anti-pruritic therapy is selected from the group consisting of moisturizers, capsaicin, salicylic acid, emollients, topical corticosteroids, topical calcineurin inhibitors, antihistamines, menthol, local anesthetics, cannabinoids, immunomodulators, antihistamines, antidepressants, μ-opiod receptor agonists, k-opiod receptor agonists, neuroleptics, substance P antagonist, immunosuppressants, methylnaltrexone, NGX-4010, TS-022, Serine proteases/PAR2 antagonists, IL-31 antibody, IL-4-receptor antibody, IL-13 antibody, TSLP-antibody, IL-5 antibody, and combinations thereof.

20. The method of claim 19, wherein the anti-pruritic therapy comprises an immunomodulator.

21. The method of claim 20, wherein the immunomodulator comprises a TRPV4 inhibitor.

22. The method of any one of claims 1-21, wherein the subject is a mammal.

23. The method of any one of claims 1-2 and 4-22, wherein the anti-pruritic therapy comprises a TRPV4 inhibitor.

24. The method of claim 23, wherein the TRPV4 inhibitor binds to a C-terminal region of TRPV4.

25. The method of claim 24, wherein the TRPV4 inhibitor binds at least one amino acid in a motif comprising K750-W772 of Xenopus TRPV4 or K754-W776 of mammalian TRPV4 or an amino acid corresponding thereto.

26. The method of claim 24, wherein the TRPV4 inhibitor binds at least one amino acid in a motif comprising R742-W772 of Xenopus TRPV4 or R746-W776 of mammalian TRPV4 or an amino acid corresponding thereto.

27. The method of claim 24, wherein the TRPV4 inhibitor binds at least one amino acid in a motif comprising K750-W772 and R742 of Xenopus TRPV4 or K754-W776 and R746 of mammalian TRPV4 or an amino acid corresponding thereto.

28. The method of claim 24, wherein the TRPV4 inhibitor binds Arg-746 of mammalian TRPV4 or Arg-742 of Xenopus TRPV4 or an amino acid corresponding thereto.

29. The method of claim 24, wherein the TRPV4 inhibitor binds at least one amino acid selected from K754, R757, R774, and W776 of mammalian TRPV4 or an amino acid corresponding thereto.

30. A method of screening for a compound that modulates TRPV4, the method comprising:
testing a plurality of compounds for binding to wild-type TRPV4 to determine from the plurality of compounds a subset of compounds that bind wild-type TRPV4; and
testing the subset of compounds that bind wild-type TRPV4 for binding to at least one mutant TRPV4, wherein the mutant TRPV4 comprises a mutation of at least one amino acid in the motif corresponding to K746-W776 of mammalian TRPV4, or an amino acid corresponding thereto, to determine from the subset of compounds a compound that binds wild-type TRPV4 but not the mutant TRPV4.

31. The method of claim 30, wherein at least one amino acid in the motif corresponding to K754-W776 of mammalian TRPV4 is mutated to an alanine.

32. The method of claim 30, wherein at least one amino acid in the motif corresponding to K754-W776 of mammalian TRPV4 is mutated to a glycine.

33. The method of claim 30, wherein at least one amino acid selected from K754, R757, R774, and W776 of mammalian TRPV4, or an amino acid corresponding thereto, is mutated.

34. The method of any one of claims 30-33, wherein the mutant TRPV4 has activity as an ion channel.

35. The method of any one of claims 30-34, further comprising determining the effect of the compound that binds wild-type TRPV4 but not the mutant TRPV4 on the activity of wild-type TRPV4.

36. The method of any one of claims 30-35, wherein the compound that binds wild-type TRPV4 but not the mutant TRPV4 inhibits the activity of wild-type TRPV4.

37. The method of any one of claims 30-35, wherein the compound that binds wild-type TRPV4 but not the mutant TRPV4 increases the activity of wild-type TRPV4.

38. The method of any one of claims 30-35, wherein the compound that binds wild-type TRPV4 but not the mutant TRPV4 inhibits or reduces the binding of LCP to wild-type TRPV4.
